(19) United States
(12) Patent Application Publication
(10) Pub. No.: US 2009/0117593 A1
(43) Pub. Date: May 7, 2009

(54) T. Cruzi-derived neurotrophic agents and methods of use therefor

(75) Inventors: Marina Chuenkova, Boston, MA (US); Miercio Pereira Perrin, Chestnut Hill, MA (US)

Correspondence Address: MCDERMOTT WILL & EMERY LLP, 28 STATE STREET, BOSTON, MA, 02109-1775, US

(73) Assignee: Tufts University, Boston, MA (US)

(21) Appl. No.: 11/982,371

(22) Filed: Nov. 1, 2007

Related U.S. Application Data
(60) Provisional application No. 60/856,170, filed on Nov. 2, 2006.

(51) Int. Cl. C07K 14/435; C07K 1/00; C12N 5/06; G01N 33/554

(52) U.S. Cl. 435/7.21; 530/326; 530/322; 435/375

(57) ABSTRACT

The invention relates to T. cruzi trans-sialidase (TS) and to the neurotrophic and IL-6 secretion-inducing activities of the protein. TS, neurotrophic variants and/or neurotrophic peptides based upon the sequence of TS can be administered to a mammal to directly or indirectly provide neurotrophic support for neurons. A mammalian neurotrophic factor (e.g., CNTF, LIF) can be co-administered with the TS, neurotrophic variant and/or neurotrophic peptide. TS, IL-6 secretion-inducing variants and/or IL-6 secretion-inducing peptides based upon the sequence of TS can be administered to a mammal to induce the secretion of IL-6. TS, active variants and/or active peptides can be administered to a mammal having an acquired or congenital condition characterized by neuronal degeneration or to a mammal that has experienced trauma to the brain, spinal cord or peripheral nerves. The invention also relates to neurotrophic and IL-6 secretion-inducing variants of TS and to neurotrophic and IL-6 secretion-inducing peptides. The invention also relates to compositions comprising TS, active variants thereof and/or active peptides and a physiologically acceptable carrier.

### RELATED APPLICATION

This application claims the benefit of U.S. Provisional Application No. 60/856,170, filed on Nov. 2, 2006. The entire teachings of the above application are incorporated herein by reference.

### GOVERNMENT SUPPORT

The invention was supported, in whole or in part, by grants ROI AI24837 and AI40574 from the National Institutes of Health. The Government has certain rights in the invention.

### BACKGROUND OF THE INVENTION

Neuronal degeneration and death normally occur during development and result in the elimination of cells which fail to make crucial inter-neural or neuro-muscular contacts. Neuronal degeneration and death also occur during senescence and as a result of pathological events (e.g., infections, acute trauma) and some genetic diseases (e.g., Huntington's disease).

Neurotrophic factors are a group of proteins that can regulate the survival, development, differentiation and many of the functions of neuronal cells. Several neurotrophic factors have been described including members of the NGF-family of neurotrophins, such as nerve growth factor (NGF), brain-derived neurotrophic factor neurotrophins, such as nerve growth factor (NGF), brain-derived neurotrophic factor (BNGF), neurotrophin-3 (NT-3) and neurotrophin-4 (NT-4), and members of the IL-6 family, including interleukin-6 (IL-6), interleukin-11 (IL-11), leukemia inhibitory factor (LIF), cilliary neurotrophic factor (CNTF) and oncostatin-M (OSM). The discovery of neurotrophic factors lead to the possibility that these factors could be administered to mammals as therapeutic agents to treat or prevent neuronal degeneration. However, the proteins which have been identified as neurotrophic factors generally mediate multiple biological functions (e.g., immune regulation, hematopoiesis). Thus, the ability to safely administer neurotrophic factors to mammals is severely limited due to undesirable side effects. For example, the administration of CNTF can result in muscle atrophy, cachexia and anorexia (Martin, D. et al., Am. J. Physiol., 27:1422-1428 (1996)).

One example of an infectious disease which leads to neuronal degeneration is Chagas' disease, which is produced by the obligate intracellular protozoan Tyrpanosoma cruzi. This disease is an important cause of cardiac and gastrointestinal (GI) morbidity and mortality in millions of people in Latin America. The disease is usually transmitted to man by infected reduviid bugs or by blood transfusion. For a few months parasites circulate in the bloodstream as a result of their invasion of, and rapid replication in a variety of cell types, particularly muscle cells in the heart and GI tract, and glial cells in the nervous system (acute infection). Most patients survive the acute infection to enter a subclinical, asymptomatic stage that lasts years to decades (the indeterminate phase). The vast majority of patients in the indeterminate phase (˜90%) show no signs of peripheral neuropathy (Genovese, O. et al., Arq. Neuropsiquiatr., 54:190-196 (1996)). In fact, the average numbers of both cardiac and GI ganglia actually increase with the age of the chagasic patient (Köberle, F., Adv. Parasitol., 6:63-116 (1968)).

The relative increase in the number of neuronal cells observed in infected individuals is dramatically different from the age-related physiological reduction in the number of cells found in ganglia of normal uninfected individuals (Köberle, F., Adv. Parasitol., 6:63-116 (1968); Meciano Filho, J., Genontology, 41:18-21 (1995)). The neuroprotective/neuroproliferative effect of T. cruzi infection in humans, is consistent with histological and electrophysiological findings in laboratory animals infected with the trypanosome. For example, infected mice showed signs of neuron development, axon regeneration and axon sprouts, in addition to some neuron degeneration. Furthermore, studies with rats infected with T. cruzi provide pharmacological evidence for axonal regrowth and sprouting in sympathetic and parasympathetic nerve fibers of the heart and colon (Machado, C. et al., Am. J. Trop. Med. Hyg., 27:20-24 (1978); Machado, C. et al., Braz. J. Med. Biol. Res., 20:697-702 (1987)).

In contrast to indeterminate phase, extensive destruction of the autonomous nervous system in the heart and GI tract occurs in individuals with chronic Chagas' disease. Histologically, the neurons in the heart are shrunk and disintegrated, with or without perineural and intraneural inflammation. This neurological pathology likely contributes to the generation of cardiomegaly (Mott, K. and Hagstrom, J., Circulation, 31:273-286 (1965); Oliveira, J. et al., Am. Heart J, 109:304-308 (1985)). In the GI tract, myenteric (Auerbach's) and submucosal (Meissner's) ganglia can be more than 95% destroyed (Köberle, F., Adv. Parasitol., 6:63-116 (1968)). This neuronal destruction provides one explanation for the tremendous enlargement of the esophagus and colon (megaesophagus and megacolon) of chronic Chagas' disease (Köberle, F., In Ciba Foundation Symposium, 20:137-147 (1974)).

The biochemical pathways that rescue neurons from death in the indeterminate phase are unknown, as are the pathways that drive neurons to die in the chronic disease. An intriguing possibility is that T. cruzi secretes a factor(s) that promote(s) development and survival of neurons. Such a factor(s) could help neurons counterbalance neurotoxic insults resulting from the infection process.

A need exists for a method of providing neurotrophic support in a mammal, and for neurotropic factors which significantly reduce or eliminate the above-mentioned problems.

### SUMMARY OF THE INVENTION

The invention relates to T. cruzi trans-sialidase (TS) and to the neurotrophic and IL-6 secretion-inducing activities of the protein. In one aspect, the invention relates to a method of providing trophic support for neurons and/or glial cells (e.g., Schwann cells) in a mammal (e.g., a human, Homo sapiens), comprising administering to the mammal a therapeutically effective amount of TS or a neurotrophic variant thereof. In one embodiment, a synergistic amount of a mammalian neurotrophic factor, such as ciliary neurotrophic factor (CNTF) or leukemia inhibitory factor (LIF), is co-administered with TS or a neurotrophic varient thereof. The neurotrophic variant can comprise the amino acid sequence of peptide C44 (SEQ ID NO:12) or of peptide C14 (SEQ ID NO:14). The neurotrophic variant can also be a fusion protein comprising TS or a neurotrophic variant thereof as a first moiety and a suitable fusion partner as a second moiety. In one embodiment, the fusion protein comprises a fusion partner which is a mammalian (e.g., human) neurotrophic factor. In an additional embodiment, TS or a neurotrophic varient thereof is administered to a mammal having a condition selected from the group consisting of amyotrophic lateral sclerosis, Alzheimer's disease, Parkinson's disease, Huntington's disease, Chagas' disease, peripheral neuropathy, palsies (e.g., cerebral, facial, Bell's, bulbar, gaze, oculomotor, progressive supranuclear, trochler), multiple sclerosis, stroke, brain trauma, spinal cord trauma and peripheral nerve trauma.

In another embodiment, the invention is a method of providing trophic support for neurons and/or glial cells in a mammal (e.g., a human), comprising administering to the mammal a therapeutically effective amount of a peptide comprising the amino acid sequence of peptide C14 (SEQ ID NO:14) or a neurotrophic varient thereof. In one embodiment, the peptide further comprises an amino-protecting group, a carboxyl-protecting group or a combination thereof. In another embodiment, the peptide is co-administered with a synergistic amount of a mammalian neurotrophic factor, such as CNTF or LIF.

In another aspect, the invention relates to a method of inducing the secretion of IL-6 in a mammal (e.g., a human), comprising administering to the mammal a therapeutically effective amount of TS or an IL-6 secretion-inducing variant thereof. In one embodiment, the variant comprises an amino acid sequence in which the amino acid sequence of peptide TR1 (SEQ ID NO:32) occurs at least twice. In another embodiment, the variant is a fusion protein, comprising TS or an IL-6 secretion-inducing variant thereof as a first moiety and a suitable fusion partner as a second moiety.

In another embodiment, the invention is a method of inducing the secretion of IL-6 in a mammal (e.g., a human), comprising administering to the mammal a therapeutically effective amount of a peptide comprising an amino acid sequence in which the amino acid sequence of peptide TR1 (SEQ ID NO:32) or an IL-6 secretion-inducing variant thereof occurs at least twice. In an additional embodiment, the peptide further comprises an amino-terminal protecting group, a carboxyl-terminal protecting group or a combination thereof.

In another aspect, the invention is a neurotrophic peptide. In one embodiment, the peptide comprises the amino acid sequence of peptide C14 (SEQ ID NO:14) or a neurotrophic variant thereof. In another embodiment, the invention is a fusion protein comprising the amino acid sequence of peptide C14 (SEQ ID NO:14) or a neurotrophic variant thereof and a suitable fusion partner. The invention also relates to a composition comprising a neurotrophic peptide or neurotrophic fusion protein and a physiologically acceptable carrier. In one embodiment, the composition comprises a peptide comprising the amino acid sequence of peptide C14 (SEQ ID NO:14) or a neurotrophic variant thereof. In another embodiment, the composition comprises a fusion protein comprising the amino acid sequence of peptide C14 (SEQ ID NO:14) or a neurotrophic variant thereof and a suitable fusion partner. In additional embodiments, the composition further comprises a mammalian neurotrophic factor, such as CNTF or LIF.

In another aspect, the invention is an IL-6 secretion-inducing peptide. In one embodiment, the peptide comprises an amino acid sequence in which the amino acid sequence of peptide TR1 (SEQ ID NO:32) or an IL-6 secretion-inducing variant thereof occurs at least twice. In another embodiment, the invention is a fusion protein comprising an amino acid sequence in which the amino acid sequence of peptide TR1 (SEQ ID NO:32) or an IL-6 secretion-inducing variant thereof occurs at least twice, and a suitable fusion partner. The invention also relates to a composition comprising an IL-6 secretion-inducing peptide or an IL-6 secretion-inducing fusion protein and a physiologically acceptable carrier. In one embodiment, the composition comprises a peptide comprising an amino acid sequence in which the amino acid sequence of peptide TR1 (SEQ ID NO:32) or an IL-6 secretion-inducing variant thereof occurs at least twice. In another embodiment, the composition comprises a fusion protein having an amino acid sequence in which the amino acid sequence of peptide TR1 (SEQ ID NO:32) or an IL-6 secretion-inducing variant thereof occurs at least twice, and a suitable fusion partner. In additional embodiments, the composition further comprises a mammalian neurotrophic factor, such as CNTF or LIF.

The invention also relates to a neurotrophic peptide comprising the amino acid sequence of peptide C19Y21 (SEQ ID NO:15) or a neurotrophic variant thereof. In some embodiments, the neurotrophic variant has at least about 85%, at least about 90%, or at least about 95% amino acid sequence identity with SEQ ID NO:15 over the length of SEQ ID NO:15.

The invention also relates to a macromolecular complex comprising a macromolecule that is covalently bonded to a neurotrophic peptide. In some embodiments, the neurotrophic peptide comprises the amino acid sequence of peptide C19Y21 (SEQ ID NO:15) or a neurotrophic variant thereof. In certain embodiments, the macromolecule is a protein, a peptide, a nucleic acid, or a polyethylene glycol.

The invention also relates to a method of activating TrkA receptor comprising combining a cell that expresses TrkA receptor with a peptide comprising the amino acid sequence of peptide C19Y21 (SEQ ID NO:15) or a neurotrophic variant thereof or a macromolecular complex of the invention, under conditions suitable for binding to TrkA receptor.

The invention also relates to a method for identifying an agent that activates TrkA receptor. In some embodiments, the method comprises combining a cell that expresses TrkA receptor and an agent to be tested under conditions suitable for binding of a ligand to said TrkA receptor, and detecting or measuring TrkA receptor activation, wherein TrkA receptor activation relative to a suitable control is indicative that the agent activates TrkA receptor, with the proviso that the agent is not nerve growth factor. In preferred embodiments, the agent to be tested competitively inhibits binding of TS to TrkA receptor, but does not competitively inhibit binding of nerve growth factor to TrkA receptor. In other preferred embodiments, the agent to be tested competitively inhibits binding of TS to TrkA receptor and competitively inhibits binding of a peptide consisting of the amino acid sequence of SEQ ID NO:15 to TrkA receptor, but does not competitively inhibit binding of nerve growth factor to TrkA receptor. In particular embodiments, TrkA receptor activation is detected or measured by detecting or measuring agent-induced TrkA dimerization, agent-induced TrkA phosphorylation, agent-induced Erk phosphorylation, agent-induced Akt phosphorylation, agent-induced CREB phosphorylation, or agent-induced cAMP response element-depended transcription.

In an additional embodiment, the invention is a composition comprising TS or a neurotrophic variant thereof, a mammalian neurotrophic factor and a physiologically acceptable carrier.

### BRIEF DESCRIPTION OF THE DRAWINGS

FIGS. 1A-B illustrate the nucleotide sequence of the T. cruzi trans-sialidase gene, clone 19Y (SEQ ID NO:1) deposited in GenBank under accession number AJ002174, having an open-reading frame beginning at position 370. The entire teachings of each of the information deposited in GenBank under accession number AJ002174 are incorporated herein by reference.

FIG. 1C illustrates the amino acid sequence of the T. cruzi trans-sialidase (SEQ ID NO:2) encoded by clone 19Y deposited in GenBank under accession number AJ002174.

FIG. 2A is a photomicrograph of PC12 cells cultured on collagen coated dishes for 16 hours in serum-free RPMI-1640/0.2% bovine serum albumin (BSA). The photomicrograph shows that PC12 cells do not extend neurites when cultured under these conditions. Magnification 400×.

FIG. 2B is a photomicrograph of PC12 cells cultured on collagen coated dishes for 16 hours in serum-free RPMI-1640/0.2% BSA supplemented with NGF (0.1 μg/ml). The photomicrograph shows that NGF induces neurite outgrowth in PC12 cells. Magnification 400×.

FIG. 2C is a photomicrograph of PC12 cells cultured on collagen coated dishes for 16 hours in serum-free RPMI-1640/0.2% BSA supplemented with V. cholera neuraminidase (VCNA)(2.4 μg/ml). The photomicrograph shows that VCNA does not induce neurite outgrowth in PC12 cells. Magnification 400×.

FIG. 2D is a photomicrograph of PC12 cells cultured on collagen coated dishes for 16 hours in serum-free RPMI-1640/0.2% BSA supplemented with TS (1 μg/ml). The photomicrograph shows that TS induces neurite outgrowth in PC12 cells. Magnification 400×.

FIG. 2E is a graph showing that TS induces neurite outgrowth in PC12 cells. The graph presents data from triplicate samples of the cultures shown in FIGS. 2A-2D. The experiment was repeated more than ten times.

FIG. 3A is a graph showing that neurite outgrowth in N18 cells is induced by immobilized laminin (Ln), immobilized TS and immobilized fragments of TS. Proteins were immobilized by incubating plates which contained wells filled with solutions of the proteins (protein concentration was 500 μg/ml) at 4° C. overnight. The protein solutions were removed and the plates were further incubated with 1% BSA at room temperature for one hour. N18 cells were cultured in the coated wells in serum-free RPMI-1640/0.2% BSA for 17 hours, and neurite outgrowth was measured by phase-contrast microscopy. Cells exhibiting neurite outgrowth were those having one or more cytoplasmic extension >2 μm in length. Ln, laminin; TS, purified T. cruzi trans-sialidase; 7F, recombinant TS from clone 7F; 19Y, recombinant TS from clone 19Y; TS-F, recombinant fragment consisting of amino acid residues 33-666 of TS (SEQ ID NO:2); TS-CC-47, recombinant fragment consisting of amino acid residues 79-478 of TS (SEQ ID NO:2); TS-Cat-47, recombinant fragment consisting of amino acid residues 79-415 of TS (SEQ ID NO:2); BSA, bovine serum albumin.

FIG. 3B is a schematic diagram of the linear structure of TS from T. cruzi clone 19Y and recombinant fragments thereof. The biological activities indicated on the right of the diagram are: trans-sialidase activity (TS), neurite extension inducing activity (NE) and antiapoptotic activity (a-AP). Numbers under the TS diagram represents amino acid number of TS from clone 19Y (GenBank accession number AJ002174). Trans-sialidase activity, neurite extension activity and antiapoptotic activities were measured at 0.1 μg/ml of purified polypeptides. (+) indicates that the polypeptide displayed significant dose-dependent extension of neurites or anti-apoptosis activity under the conditions tested. (−) indicates that the polypeptide was inactive.

FIG. 3C is a graph showing that synthetic peptide C44 (SEQ ID NO:12) or synthetic peptide C14 (SEQ ID NO:14) induce dose dependent outgrowth of neurites in PC12 cells cultured on collagen coated dishes.

FIGS. 4A and 4B are series of photomicrographs showing that TS inhibits trophic factor withdrawal-induced apoptosis of PC12 cells. PC12 cells were cultured on collagen-coated plastic dishes in serum-free RPM/0.2% BSA supplemented with various reagents for 17 hours. Apoptotic cells were detected by staining with 4,6-diamino-2-phenylindole (DAPI) (FIG. 4A) or by DNA nick end-labeling (TUNEL) (FIG. 4B). The additions in FIG. 4A were: a) no addition; b) 0.1 μg/ml NGF; c) 2.4 μg/ml VCNA; d) 0.1 μg/ml TS; e) 0.1 μg/ml TS-F-47 (amino acid residues 79-666 of SEQ ID NO:34; f) 0.1 μg/ml TS-CC-47; g) 2 μg/ml peptide CF1; h) 2 μg/ml peptide CY1; and i) 0.1 μg/ml TS-Cat-47. Additions in FIG. 4B were: a) no addition; b) 0.1 μg/ml NGF; c) 0.1 μg/ml TS; d) 0.1 μg/ml TS-F-47; e) 0.1 μg/ml TS-CC-47; f) 0.1 μg/ml TS-Cat-47. Magnification, 1000×.

FIG. 5A is a graph showing that TS protects PC12 cells from apoptosis induced by trophic factor withdrawal in a dose dependent manner. PC12 cells were cultured in serum-free RPMI-1640/0.2% BSA or in the same medium supplemented with TS, NGF or VCNA for 17 hours. The molar concentration of TS, NGF and VCNA was based on a MW of 200 kDa for TS, 26 kDa for NGF, and 90 kDa for VCNA. Apoptosis was measured by staining the cells with DAPI.

FIG. 5B is a graph showing that TS delayed trophic factor withdrawal-induced apoptotic death of PC12 cells. PC12 cells were cultured in serum-free RPMI-1640/0.2% BSA supplemented with TS (0.5 nM) or NGF (4.0 nM). Cell viability was measured by determining the number of cells without nuclear fragmentation relative to those with nuclear fragmentation by staining with DAPI. 300-400 cells were analyzed in each sample. The presented data are the average of two tests in which each data point represents the average of 3 determinations.

FIG. 6A is a graph showing that TS and certain fragments of TS protected PC12 cells from apoptosis induced by trophic factor withdrawal in a dose dependent manner. PC12 cells were cultured in serum-free RPMI-1640/0.2% BSA in the presence of the indicated concentration of TS or recombinant TS polypeptides for 17 hours. Apoptosis was measured by staining the cells with DAPI.

FIG. 6B is a graph showing that synthetic peptides C14 (SEQ ID NO:14) and C44 (SEQ ID NO:12) protected PC12 cells from apoptosis induced by trophic factor withdrawal. PC12 cells were cultured in serum-free RPMI-1640/0.2% BSA supplemented with synthetic peptides (1 μM) for 17 hours. Apoptosis was measured by staining the cells with DAPI. Peptide B2 (SEQ ID NO:18), Peptide TR (SEQ ID NO:19).

FIG. 7 is a photograph of an ethidium bromide stained electrophoresis gel showing that TS induces the expression of Bcl-2 mRNA in PC12 cells. PC12 cells were cultured for 17 hours in serum-free RPMI-1640/0.2% BSA (RPMI) and in the same medium supplemented with NGF (100 ng/ml) or TS (100 and 200 ng/ml). Gene expression was assessed by RT-PCR analysis of total RNA using primers which specifically amplified Bcl-2 or GAPDH cDNA.

FIG. 8A is a graph showing synergy between TS and CNTF or LIF in protecting PC12 cells from apoptosis induced by trophic factor withdrawal. PC12 cells were cultured for 17 hours in serum-free RPMI-1640/0.2% BSA (negative control, 0% protection), or in the same medium supplemented with the indicated cytokines. TS was used at a concentration of 10 pM (2.5 ng/ml), CNTF was used at 2 nM (50 ng/ml), LIF was used at 1 ng/ml and all other cytokines were used at 5 nM. Apoptosis was measured by DAPI staining.

FIG. 8B is a graph showing the dose-response of the synergistic anti-apoptotic action of TS with CNTF. PC12 cells were cultured for 17 hours in serum-free RPMI-1640/0.2% BSA (0% protection), in the same medium supplemented with the indicated concentration of TS, or in medium supplemented with 50 ng/ml CNTF and with the indicated concentrations of TS (TS+CNTF). Apoptosis was measured by DAPI staining.

FIG. 8C is a graph showing that quantities of TS and CNTF which did not induce bcl-2 gene expression individually were synergistic, and dramatically induced the expression of bcl-2 mRNA. PC12 cells were cultured in serum-free RPMI-1640/0.2% BSA medium (RPMI) and the same medium supplemented with TS (2.5 ng/ml), CNTF (50 ng/ml) or TS (2.5 ng/ml)+CNTF (50 ng/ml). Total RNA was isolated 17 hours after addition of the growth factors and bcl-2 gene expression was assessed by RT-PCR. The amplified Bcl-2 transcripts in the agarose gels were quantified using a scanning densitometer and normalized to the expression of the GAPDH gene.

FIG. 8D is a graph illustrating the dose-response relationship of the synergistic anti-apoptotic action of TS with LIF. PC12 cells were cultured for 17 hours in serum-free RPMI-1640/0.2% BSA (0% protection), in the same medium supplemented with 2.5 ng/ml of TS, or in medium supplemented with 2.5 ng/ml of TS which further contained the indicated concentrations of LIF or IL-11 (TS+LIF and TS+IL-11).

FIG. 9 is a graph showing dose-dependent inhibition of serum withdrawal-induced apoptosis of human Schwann cells by TS. Immortalized Schwann cells (Rambukkana, A. et al., Science, 282:2076-2079 (1998)) which had been maintained in DMEM supplemented with 10% fetal calf serum were washed 3 times with serum-free DMEM. The cells were then cultured in serum-free DMEM or in serum-free DMEM containing recombinant TS. After culture for 24, 48 or 72 hours, cells were fixed in 4% formaldehyde in phosphate buffered saline (PBS) for 10 minutes, washed with PBS and stained with DAPI (5 μg/mL) for 2 minutes. Cells with fragmented nuclei (apoptotic cells) were quantified by inspection of 300-400 cells using a fluorescence microscope.

FIGS. 10A-10F are photomicrographs of primary granule neurons. The neurons were maintained in basal Eagle medium (BME) containing high K⁺ (25 mM KCl) supplemented with 10% FCS (FIGS. 10B and 10E). After 7 days, the medium was changed to serum-free BME containing low K⁺ (5 mM KCl) (FIGS. 10A and 10D), or to serum-free BME containing low K⁺ and TS (FIGS. 10C and 10D) and the cells were cultured for a further 24 hours. Then, the cells were stained with fluorescein diacetate (FDA). FIGS. 10A-10C are photomicrographs taken under phase contrast illumination. FIGS. 10D-10F are photomicrographs of cells that were stained with FDA taken under UV illumination. Under these conditions, viable cells are fluorescent. Note the increased number of viable cells in medium containing high K⁺ or low K⁺ supplemented with TS (100 ng/ml) (FIGS. 10E and 10F). Original magnification, 400×.

FIG. 10G is a graph which shows the percentage of viable cells found in cultures of granule neurons. Granule neurons were maintained in BME medium containing high K⁺ (25 mM KCl) supplemented with 10% FCS (FIGS. 10B and 10E) for 7 days. Then the medium was changed to serum-free BME containing low K⁺ (5 mM KCl), or to serum-free BME containing low K⁺ and TS (100 ng/ml) or the catalytic domain of TS (TS-F, 100 ng/ml) and the cells were cultured for a further 24 hours. Control cells were cultured for the further 24 hours in high K⁺ media (25 mM KCl). Viable cells were quantified by staining with FDA and fluorescence microscopy.

FIGS. 11A and 11B are graphs showing reversal of the survival-promoting activity of TS by Wortmannin (FIG. 11A) or LY294002 (LY, FIG. 1B), which are inhibitors of PI-3 kinase. Cultures of PC12 were switched to serum-free RPMI or to serum-free RPMI supplemented with a polypeptide consisting of the catalytic domain of TS (TS-F, 100 ng/ml), NGF (100 ng/ml), CNTF (50 ng/ml) or TS-F (5 ng/ml)+CNTF (50 ng/ml) and cultured for 24 hours. Then, Wortmannin or LY was added to the cultures at the indicated concentrations. Neuronal cell viability was measured 24 hours later by staining with DAPI. Viability of PC12 cells cultured in RPMI alone (33%) was similar to the viability of PC12 cells cultured in RPMI+CNTF.

FIG. 11C is a photograph of a Western blot showing that TS induces serine phosphorylation of Akt kinase, and a graph showing the quantities of serine phosphorylated Akt kinase detected in the Western blot. PC12 cells were washed in serum-free RPMI and kept in RPMI containing 0.1% FCS. After 2 days, the medium was changed to serum free medium for two hours. Then the cells were challenged with 10% FCS (serum), serum free media (RPMI) or TS (100 ng/ml) for 2, 5 or 10 minutes. Cells were lysed in 2% sodium dodecylsulfate (SDS) and proteins in the lysates were resolved by SDS-PAGE through 10% gels. The proteins were transferred to nitrocellulose membranes and probed using an antibody which specifically binds to Akt kinase that is phosphorylated on Ser 473 (phospho-Akt antibody, New England BioLabs, Beverly, Mass.). The bands detected by the antibody were quantified by scanning densitometry.

FIG. 11D is a photograph of a Western blot showing that the PI-3 kinase inhibitor LY294002 (LY) prevents TS-induced serine phosphorylation of Akt kinase in PC12 cells. PC12 cells were washed in serum-free RPMI and kept in RPMI containing 0.1% FCS. After 2 days, the medium was changed to serum free medium for two hours. Then the cells were challenged with 10% FCS (serum), serum free media (RPMI) or TS (100 ng/ml) for 2, 5 or 10 minutes. An additional group of cells were preincubated with LY (1 μM) prior to the addition of the catalytic domain of TS (TS-F, 100 ng/ml). Cells were lysed in 2% sodium dodecylsulfate (SDS) and proteins in the lysates were resolved by SDS-PAGE through 10% gels. The proteins were transferred to nitrocellulose membranes and probed using an antibody which specifically binds to Akt kinase that is phosphorylated on Ser 473 (phospho-Akt antibody, New England BioLabs, Beverly, Mass.). LY completely blocked TS-F-induced phosphorylation of Akt kinase. LY was similarly effective in blocking TS-induced phosphorylation of Akt kinase in PC12 cells.

FIGS. 12A-12C are graphs showing dose dependent release of IL-6 by cells stimulated with TS, VCNA and penetrin (PN-1). HIMEC cells (FIG. 12A), PBMC (FIG. 12B) or T-24 carcinoma cells (FIG. 12C) were cultured for 24 hours in media containing various concentrations of TS, VCNA or PN-1. The supernatants of the cultures were then assayed for IL-6 and other cytokines by ELISA. The quantity of TS or PN-1 added to the culture media is expressed as μg/ml, the quantity of VCNA added to the culture media is expressed as mU/ml (the unit corresponded to the neuraminidase activity of TS). The presented data are representative of at least 9 independent studies.

FIGS. 12D and 12E are graphs showing the time course of IL-6 secretion by TS stimulated cells. HIMEC cells (FIG. 12D) or PBMC (FIG. 12E) were cultured in media containing TS (1 μg/ml) or VCNA (1 mU/ml). After culture for predetermined amounts of time, the supernatants of the cultures were assayed for IL-6.

FIG. 13A is a photograph of an ethidium bromide stained agarose gel showing that TS stimulation induces expression of IL-6 mRNA in PBMC. PBMC were cultured in media (M) or media containing 1 μg/ml of TS (T) for 4, 10 or 24 hours. Gene expression was assessed by RT-PCR analysis of total RNA using primers which specifically amplified IL-6 or GAPDH cDNA.

FIG. 13B is a graph showing that TS stimulation induces expression of IL-6 mRNA in PBMC. Expression of IL-6 mRNA relative to GAPDH mRNA was determined by densitometric measurement of the bands in FIG. 13A.

FIG. 14 is a graph showing that TS-conditioned medium restores growth of the IL-6-dependent B-lymphoma, DS-1. PBMC were cultured in media (CM) or in media supplemented with TS (TS/CM) for 24 hours. The conditioned media from these cultures were diluted into IL-6 free media at 1:40, 1:20, 1:10 and 1:5, and DS-1 cells were cultured in the resulting mixture. DS-1 cell proliferation was assessed by measuring the amount of ³H-thymidine incorporated into the DNA of cells. TS/CM induced dose dependent proliferation of DS-1 cells. Addition of a neutralizing rabbit anti-IL-6 IgG (1 μg/ml, TS/CM+IL-6 Ab) blocked DS-1 cell proliferation in response to TS/CM, but normal rabbit IgG had no effect on the TS/CM-induced proliferation. IL-6 free media supplemented with 1 μg/ml TS (TS), which corresponds to 1:5 dilution of conditioned media, did not induce proliferation of the DS-1 cells.

FIG. 15A is a schematic diagram of TS clone 7F and of recombinant fragments TS-154 and TS-H32. NU-17, NU-18, NU-19 and NU-20 are synthetic primers used to make constructs encoding recombinant fragments TS-154 and TS-H32. H6 is a 6×His tag. The amino acid sequences encoded by the Bgl II/Pst I fragments of constructs TS-154 (SEQ ID NO:30) and TS-H32 (SEQ ID NO:31) are presented. The sequences are identical (identical residues indicated by (−)) except where indicated with the amino acid letter code.

FIG. 15B is a graph showing the catalytic activity of TS, the catalytic domain of TS (CD), and recombinant fragments TS-154 and TS-H32.

FIG. 15C is a graph showing the IL-6 secretion inducing activity of TS, the catalytic domain of TS (CD), and recombinant fragments TS-154 and TS-H32. The IL-6 secretion inducing activity of the polypeptides was assessed in cultures of PBMC. Similar activity was detected in cultures of T-24 cells.

FIG. 16A is a graph showing dose dependent secretion of IL-6 by T-24 cells stimulated with recombinant tandem repeat domain (LTR) of TS. T-24 cells were cultured for 24 hours in media or in media containing LTR or the catalytic domain of TS (CD). IL-6 secreted into the culture media was assessed by ELISA. Similar results were obtained in studies using PBMC.

FIG. 16B is a graph showing that immunodepletion of LTR removes IL-6 secretion-inducing activity. An LTR solution was passed through a protein G-Sepharose column adsorbed with either anti-LTR mAb TCN-2 (TCN-2) or a control anti-p-azo-phenylarsonate IgG1 (Ctrl/IgG1). The flow-through of each column was added to cultures of T-24 cells. The cells were cultured for 24 hours and the culture media was assayed for IL-6 by ELISA. Eluates from each column were obtained by mechanical stirring of the agarose followed by centrifugation and tested for IL-6 secretion inducing activity in the same way as the flow-throughs.

FIG. 17 is a graph showing synthetic peptides having amino acid sequences based upon the LTR of TS stimulate IL-6 release by PBMC. PBMC were cultured in media or in media containing a synthetic peptide at 50 mM, 100 mM or 200 mM. The synthetic peptides tested were TR1 (SEQ ID NO:32), TR2 (SEQ ID NO:26), TR3 (SEQ ID NO:27), TR4 (SEQ ID NO:28) or TR5 (SEQ ID NO:29).

FIG. 18A is a graph showing the trans-sialidase activity of unfractionated T. cruzi trypomastigotes (Unf), or TS⁺ T. cruzi trypomastigotes (TS⁺), of TS⁻ T. cruzi trypomastigotes (TS⁻) and of L. major promastigotes (L. major).

FIG. 18B is a graph showing the quantity of IL-6 released by HIMEC after infection with and of L. major promastigotes (L. major).

FIG. 18C is a series of three photomicrographs showing the morphology of TS⁻ T. cruzi trypomastigotes (TS⁻), TS⁺ T. cruzi trypomastigotes (TS⁺) or unfractionated T. cruzi trypomastigotes (Unf).

FIG. 19 is a bar graph showing that T. cruzi infection protected human Schwann cells from apoptosis. Monolayers of Schwann cells were infected with T. cruzi for 2 hours in DMEM/10% FCS and cultured for a further 72 hours in serum-free medium. To quantify apoptotic cells and infected cells, infected cell monolayers were examined by fluorescence microscopy after staining with DAPI, to reveal fragmented or condensed nuclei, and after staining with Chagasic IgG followed by Alexa 594-labeled anti-human IgG to reveal intracellular parasites. The bar graph illustrates the quantitative results.

FIG. 20A is a photograph of a Western blot showing that Akt kinase was activated in human Schwann cells infected with T. cruzi trypomastigotes. Schwann cells were grown in DMEM with 0.1% FCS for 2 days, changed to serum free medium for 1 hour, treated for 10 minutes with the indicated concentrations of invasive trypomastigotes (Tryps) and noninvasive epimastigotes (Epis), washed, lysed, and the lysates tested for Akt phosphorylation (Ser473) by Western blot. Infection with the invasive trypomastigotes resulted in the generation of phosphorylated Akt kinase (P-Akt (Ser473)).

FIGS. 20B and 20C are a photograph of a Western blot and a bar graph, respectively, showing that Akt kinase was activated in human Schwann cells infected with TS⁺ T. cruzi trypomastigotes. Schwann cells were grown as described in the legend to FIG. 20A, and challenged for 20 minutes with 1×10⁷ TS⁺ trypomastigotes (TS⁺; invasive form), TS⁻ trypomastigotes (TS⁻; noninvasive form), or unfractionated trypomastigotes. The cells were then washed, lysed, and the lysates tested for Akt phosphorylation (Ser473) by Western blot. FIG. 20B shows that TS⁺ trypomastigotes induced phosphorylation of Akt (P-Akt (Ser473)). FIG. 20C shows the trans-Sialidase activity of the trypomastigotes populations used to infect the Schwann cells (note the heterogenous distribution of TS activity in the TS⁺ and TS⁻ subsets).

FIG. 21A is a bar graph showing that the catalytic domain of TS (CD, also referred to as TS-F or ΔTS) protected human Schwann cells from apoptosis. Nearly confluent Schwann cells were detached from the substratum by trypsinization, washed with serum-free DMEM, plated in the same medium with or without additives for 72 hours. Apoptotic cells were quantified by fluorescence microscopy after staining with DAPI, to reveal fragmented or condensed nuclei. The bar graph illustrates that CD was nearly as effective as 2% serum at inhibiting apoptosis. In contrast, TSA-1 did not inhibit apoptosis in the assay.

FIG. 21B is a graph illustrating dose-dependent inhibition of serum withdrawal-induced apoptosis of human Schwann cells by CD. Immortalized Schwann cells were detached from the substratum by trypsinization, washed with serum-free DMEM, and plated in the same medium or in medium supplemented with CD at the indicated concentrations. After culture for 24, 48 or 72 hours, cells were fixed in 4% formaldehyde in phosphate buffered saline (PBS) for 10 minutes, washed with PBS and stained with DAPI (5 μg/mL) for 2 minutes. Cells with fragmented nuclei (apoptotic cells) were quantified by inspection using a fluorescence microscope.

FIG. 22A is a photograph of a Western blot showing that CD induced transient phosphorylation of Akt kinase in human Schwann cells. Schwann cells were maintained in DMEM supplemented with 0.1% FCS for 2 days, switched to serum-free DMEM medium for 1 hour and challenged with CD (500 ng/ml) for 1, 2, 5, 10 or 14 minutes, lysed in lysis buffer and tested for phosphorylation of Akt kinase by immunoblot.

FIGS. 22B and 22C are a photograph of a Western blot and a bar graph, respectively, showing that CD activated PI3K/Akt kinase signaling in human Schwann cells. Schwann cells were maintained in serum-free DMEM, in DMEM supplemented with 500 ng/ml CD for 2 minutes, or in DMEM that contained LY294002 (20 μg) for 30 minutes after which CD was added (final concentration of CD 500 ng/ml) and the cells were cultured for an additional 2 minutes (LY+CD). The resulting monolayers were fixed, stained with DAPI and the percentage of cells with fragmented (apoptotic) nuclei was calculated for each sample in triplicate. FIG. 22B shows that CD induced phosphorylation of Akt kinase (P-Akt(Ser473)) and that the CD-induced phosphorylation of Akt kinase was inhibited by the PI3 kinase inhibitor LY294002. FIG. 22C shows that CD protected Schwann cells from apoptosis and that LY294002 inhibited the protective activity of CD. The CD-induced protection of Schwann cells from apoptosis correlated with CD-induced phosphoryl of Akt kinase.

FIG. 23A is a photograph of a Western blot showing that L. major which expressed T. cruzi TS activated Akt in Schwann cells. Schwann cells were maintained as described in the legend for FIG. 22A and challenged with 10⁸/ml L. major promastigotes that were transfected with an empty vector (pXG1a) or with vector pXG1a-TS which encodes T. cruzi TS. Monolayers were washed, lysed and the lysates tested for Akt phosphorylation by immunoblot. The analysis revealed that L. major that expressed T. cruzi TS induced phosphoryation of Akt kinase (P-Akt (Ser473)).

FIG. 23B is a bar graph showing that L. major which expressed T. cruzi TS activated Akt in Schwann cells. The graph shows the relative amount of phosphorylated Akt kinase that was detected in Schwann cells challenged with 10⁸/ml L. major promastigotes transfected with an empty vector (pXG1a) or with vector pXG1a-TS which encodes T. cruzi TS, and the TS activity of L. major transfected with empty vector (PXG1a) or with vector pXG1a-TS which encodes T. cruzi TS. The results show that L. major that expressed TS induced phosphorylation of Akt kinase in Schwann cells.

FIG. 24A is a photograph of a Western blot showing that CD did not activate Akt kinase in Schwann cells that over expressed a kinase-inactive mutant Akt (AktKI). Schwann cells were transfected with a vector encoding green fluorescent protein (GFP), or with vectors encoding GFP and a dominant negative mutant Akt kinase (AktKI). The transfected Schwann cells were maintained in 0.1% FCS for 48 hours without tetracycline to induce GFP and AktKI expression (GFP and AktKI, respectively) or maintained in 0.1% FCS for 48 hours without tetracycline and then challenged for 2 minutes with CD at 500 ng/ml (GFP/CD and AktKI/CD, respectively). The cells were then lysed on ice with nonionic detergent. Akt was immunoprecipitated from the lysates with an Akt-specific antibody and its kinase activity toward GSK-3α substrate assessed by immunoblot.

FIG. 24B is a bar graph showing that CD did not promote survival of Schwann cells that over expressed a kinase-inactive mutant Akt (AktKI). Schwann cells were cultured as described in the legend for FIG. 24A, except that the cells were maintained in serum-free medium for 48 hours without (GFP and AktKI) and with 500 ng/ml CD (GFP/CD and AktKI/CD). Cells that contained apoptotic nuclei were quantified after staining with DAPI. The results presented in FIGS. 24A and 24B show that CD did not promote survival or activate Akt kinase in Schwann cells which over expressed kinase-inactive Akt (AktKI).

FIG. 25A is a photograph of a Western blot showing that CD did not induce phosphorylation of Akt kinase in Schwann cells that over expressed PTEN. Schwann cells were transfected with an empty vector pcDNA3-neo (neo), pcDNA3-neo-PTEN which encoded PTEN (PTEN) or with a vector that encoded a phosphatase-inactive pG129R PTEN mutant (pG129R). Transfected Schwann cells were grown in serum-free medium for 2 days without (neo, PTEN, pG129R) or with 500 ng/ml of CD (neo/CD, PTEN/CD, pG129R/CD). Then, the cells were lysed and the lysates examined for phosphorylation of Akt (Ser473) by Western blot. The results revealed that PTEN inhibited CD-induced phosphorylation of Akt kinase (P-Akt (Ser473)), but that an inactive mutant PTEN (PG129R) did not inhibit CD-induced phosphorylation of Akt kinase.

FIG. 25B is a bar graph showing that CD did not rescue Schwann cells that over expressed PTEN from apoptosis. Schwann cells were transfected with an empty vector pcDNA3-neo (neo), pcDNA3-neo-PTEN which encoded PTEN (PTEN) or with a vector that encoded a phosphatase-inactive pG129R PTEN mutant (pG129R) and were cultured as described in the legend to FIG. 25A, except the Schwann cell monolayers were fixed and the cells analyzed for apoptotic nuclei after DAPI staining.

FIGS. 26A-B illustrate the nucleotide sequence of the T. cruzi trans-sialidase gene, clone 7F (SEQ ID NO:33) deposited in GenBank under accession number M61732, having an open-reading frame beginning at position 484. The entire teachings of each of the information deposited in GenBank under accession number M61732 are incorporated herein by reference.

FIG. 26C illustrates the amino acid sequence of the T. cruzi trans-sialidase (SEQ ID NO:34) encoded by clone 7F deposited in GenBank under accession number M61732. TS comprises a catalytic domain (amino acid residues 33-666 of SEQ ID NO:34), and a tandem repeat domain (amino acid residues 667-1162 or SEQ ID NO:34).

FIG. 27A is a graph showing that peptide Y₂₁ (SEQ ID NO:15) induced cell survival. PC12 cells that express TrkA (trkA) or that do not (nnr5) were cultured in serum-free medium without (0% protection) or with the indicated concentrations of peptide Y₂₁ (SEQ ID NO:15). Cell viability was assessed using MTT colorimetric assay.

FIG. 27B is a histogram showing the specificity of peptide Y₂₁ (SEQ ID NO:15) responses. Cells were starved in serum-free medium without or with synthetic PDNF-derived peptides (peptide Y₂₁ (also referred to as peptide C19Y21; SEQ ID NO:15), peptide Y₁₄ (SEQ ID NO:35), peptide Y₇ (also referred to as peptide CYFN; SEQ ID NO:17), peptide TR (SEQ ID NO:19) and peptide BR (also referred to as peptide B2; SEQ ID NO:18)) (10 μM), PDNF (500 ng/ml) or NGF (30 ng/ml) for 48 hours. Apoptosis was determined by fluorescence microscopy after staining with DAPI.

FIG. 27C is a histogram showing that peptide Y₂₁ (SEQ ID NO:15) retains its cell survival promoting activity when coupled to a macromolecular carrier. Peptide Y₂₁ (SEQ ID NO:15) or peptide TR (SEQ ID NO:19) were cross-linked to BSA and assayed for promotion of PC12 cell survival for 3 and 5 days. Apoptotic cells were counted by fluorescence microscopy after staining with DAPI.

FIGS. 28A-28C are microscopy images showing that peptide Y₂₁ (SEQ ID NO:15) promotes differentiation of PC12 cells expressing TrkA. FIG. 28A: PC12^{TrkA} cells in DMEM/1% FCS were cultured for 3 days in plates coated with peptide Y₂, (SEQ ID NO:15), TR (SEQ ID NO:19) or collagen. Live cells (containing dark insoluble formazan) were visualized by Nomarski microscopy after staining with MTT. FIG. 28B: PC2^{TrkA} cells were maintained for 5 days in RPMI/1% FCS medium without or with peptide Y₂₁ (SEQ ID NO:15), TR (SEQ ID NO:19; 10 μM) or PDNF (500 ng/ml), added to the medium. FIG. 28C: PC12^{nnr5} were cultured as above without (−) or with peptide Y₂₁ (SEQ ID NO:15; 10 μM) or 5% FSC (Serum), as positive control. Morphological differentiation of PC12 cells was determined by Nomarski microscopy after MTT staining. ×400.

FIG. 29A is an image of protein blot showing that peptide Y₂₁ (SEQ ID NO:15) inhibited PDNF binding to TrkA, and a graph showing the quantitative results as determined by scanning densitometry. Purified recombinant PDNF was chromatographed by SDS/PAGE, transferred to nitrocellulose and probed with a fusion protein containing the extracellular domain of human TrkA receptor (TrkA-ECD) fused to Fc domain without pre-incubation (0) or after pre-incubation (4° C., 24 hours) with 100 nM PDNF (PDNF), or with the indicated concentrations of peptide Y₂₁ (SEQ ID NO:15). Binding of TrkA to PDNF was detected with anti-human HRP-conjugated antibody. Relative amount of TrkA-ECD bound to PDNF was determined by scanning densitometry and plotted using the program GraphPad Prism (graph).

FIG. 29B is an image of a protein blot showing that peptide Y₂₁ (SEQ ID NO:15) did not inhibit NGF binding to TrkA. The methods used were similar to those described in FIG. 3A, except that NGF, rather than PDNF, was immobilized on nitrocellulose.

FIG. 29C is an image of a protein blot showing that peptide Y₂₁ (SEQ ID NO:15) induced dimerization of TrkA. PC2^{TrkA} cells were grown in serum-free medium for 24 hours, incubated in the same medium without (No Ligand) or with peptide Y₂₁ (SEQ ID NO:15) (10 μM) or NGF (5 ng/ml) for 1 hour, and surface proteins were chemically cross-linked with bis(sulfosuccinimidyl)suberate. Cells were lysed in SDS buffer, proteins resolved by SDS/PAGE, transferred to nitrocellulose and reacted with anti-TrkA antibody to detect TrkA-TrkA homodimers (MW ˜280 kDa) (arrow).

FIG. 29D PC12^{TrkA} cells and PC12^{nnr5} cells were starved in serum-free medium for 24 hours, stimulated with NGF (30 ng/ml) and the indicated concentrations of Y₂₁. Cells were lysed, immunoprecipitated with anti-TrkA antibody and subjected to SDS/PAGE and Western blotting with phospho-TrkA antibody to identify phospho-TrkA (arrow).

FIGS. 30A and 30B are images of protein blots showing that peptide Y₂, (SEQ ID NO:15) activated MAPK/Erk signaling. FIG. 30A: PC12 cells were incubated in serum-free medium for 24 hours before stimulation for 5 minutes with the indicated concentrations of peptide Y₂₁ (SEQ ID NO:15) or with 10 μM of peptides TR (SEQ ID NO:19), Y₁₄ (SEQ ID NO:35), or Y₇(SEQ ID NO:17). Cell lysates were analyzed by Western blotting with anti-phospho-Erk antibody or with anti-Erk antibody and HRP-labeled second antibody. FIG. 30B: Left panel: PC12 cells were stimulated with peptide Y₂₁ (SEQ ID NO:15; 10 μM) for the indicated times or with NGF (30 ng/ml) for 10 minutes, and analyzed by Western blotting with antibodies against P-Erk and Erk. Right panel: PC12^{nnr5} cells were untreated (0) or treated with NGF for 10 minutes or with peptide Y₂₁ (SEQ ID NO:15) for 5 minutes, and probed with P-Erk antibody by Western blotting.

FIG. 31A is an image of a protein blot and a graph showing that peptide Y₂₁ (SEQ ID NO:15) inhibited PDNF-dependent stimulation of P-Erk. PC12 cells were serum-starved for 24 hours, treated with the indicated concentration of peptide Y₂, (SEQ ID NO:15) for 5 minutes, followed by 7 nM of PDNF for 10 minutes, lysed, and probed with anti-phospho-Erk antibody or anti-Erk-antibody by Western blotting (image). The amount of P-Erk detected was estimated by scanning densitometry (line graph). Data shown are the median of triplicate experiments.

FIG. 31B is an image of a protein blot showing that peptide Y₂₁ (SEQ ID NO:15) did not inhibit NGF-dependent stimulation of P-Erk. PC12 cells were serum starved and treated with the indicated concentration of peptide Y₂₁ (SEQ ID NO:15) as described in FIG. 31A, followed by 80 pM of NGF for 5 minutes, lysed, and probed with P-Erk antibody.

FIGS. 32A and 32B are images of protein blots showing that peptide Y₂₁ (SEQ ID NO:15) induced phosphorylation of Akt tyrosine kinase. FIG. 32A: PC12 cells were stimulated with peptide Y₂₁ (SEQ ID NO:15; 10 μM) for the indicated amount of time or with NGF (30 ng/ml, 10 minutes). Identification of P-Akt was by Western blotting. FIG. 32B: Cells were stimulated for 5 minutes with various concentrations of peptide Y₂₁ (SEQ ID NO:15) (upper panel) or with 10 μM of peptides Y₇ (SEQ ID NO:17), Y₁₄ (SEQ ID NO:35), Y₂₁ (SEQ ID NO:15), or TR (SEQ ID NO:19) (lower panel). Total cell proteins were subjected to PAGE, followed by immunoblotting with anti-phospho-Akt antibody.

FIG. 33A is an image of a protein bolt showing that peptide Y₂₁ (SEQ ID NO:15) induced phosphorylation of CREB. PC12 cells were treated with PDNF (500 ng/ml) for 10 minutes, or peptide Y21 (SEQ ID NO:15; 10 μM) or NGF (30 ng/ml) for minutes. Phosphorylated CREB (P-CREB) and CREB were detected by Western blotting with antibody specific for P-CREB or CREB, respectively.

FIG. 33B is an schematic showing the structure of pFLuc reporter plasmid with TH promoter (enhancer/promoter elements marked) driving a luciferase reporter (adopted from Lewis-Tuffin, L. et al., Mol. Cell. Neurosci., 25:536-547 (2004)), and a histogram showing that peptide Y₂₁ (SEQ ID NO:15) activated cAMP response element-dependent transcription. PC12 cells transfected with pFLuc (pFluc/TrkA and pFluc/nnr5) or with pCRE-(CRE-/TrkA) were treated with control medium (control), NGF, PDNF or peptide Y₂₁ (SEQ ID NO:15) for 10 hours. Data were normalized as fold of pFLuc expression in PC12^{trkA} under control conditions.

FIG. 34 is a schematic showing the predicted secondary structure of peptide Y₂₁ (SEQ ID NO:15), and an image showing the location of peptide Y₂₁ (SEQ ID NO:15) in the three dimensional struction of PDNF. The amino-acid sequence of peptide Y₂₁ depicting secondary structures (arrows represent β-strands and line connecting anti-parallel β-strands denotes a β-hairpin turn) that occurs in the 3D structure of intact PDNF (white arrow) is shown. Location of Y₂₁ on the 3D structure of N-terminal region of PDNF (Buschiazzo, A. et al., Mol. Cell, 10:757-768 (2002)) is also shown. The secondary structure of Y₂₁ and location of Y₂₁ in the 3D structure of PDNF were determined with the Cn3D 4.1 program (National Center for Biotechnology Information, NIH). Note that peptide Y₂₁ is located in the lectin-like domain of PDNF.

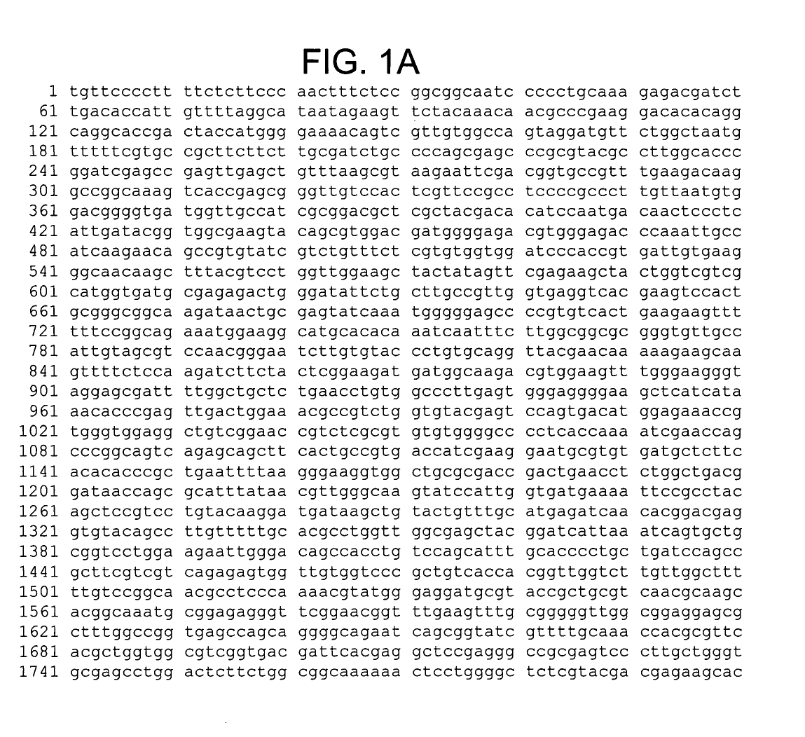

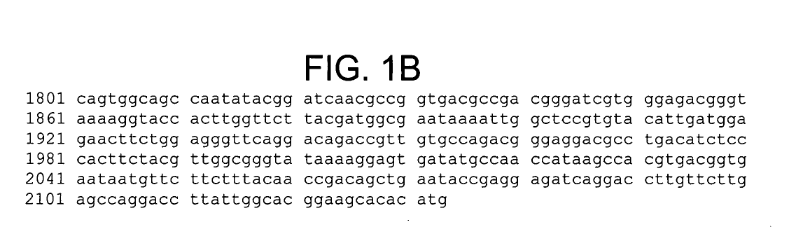

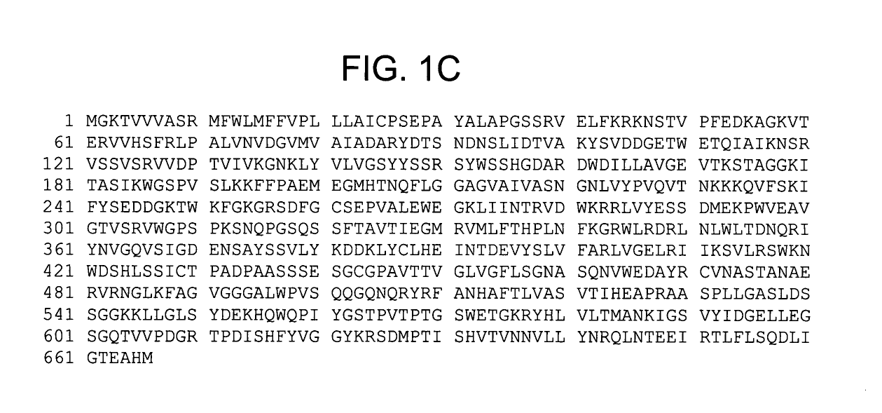

### DETAILED DESCRIPTION OF THE INVENTION

Acute Chagas' disease is characterized by robust growth of T. cruzi in several tissues throughout the body, which, in turn, can lead to severe damage of the nervous system. Humans with acute Chagas' disease, particularly young children, can suffer from fulminating encephalitis, and the autonomic nervous system is partially destroyed in experimental animals with acute T. cruzi infection (Alcantara, F., Parasit., 10:296-301 (1959); Tafuri, W., Am. J. Trop. Med. Hyg., 19:405-417 (1970); de Souza, M., et al., Mem. Inst. Oswaldo Cruz, 91:217-224 (1996)). The disease progresses from the acute phase to the indeterminate phase, which is distinguished by very low tissue parasitism, absence of symptoms, and paucity of lesions in the nervous system and other organs. Therefore, neuron regeneration must occur when Chagas' disease progresses from the acute disease to the indeterminate phase. Evidence for such neuron regeneration includes an increase in neurotransmitters in the heart, GI tract, and salivary glands (Machado, C. et al., Am. J. Trop. Med. Hyg., 27:20-24 (1978); Tanowitz, H. et al., Exp. Parasitol., 51:269-278 (1981); Machado, C. et al., Braz. J. Med. Biol. Res., 20:697-702 (1987)); motor unit remodeling; axonal sprouts; and remyelination in peripheral nerves (Gonzalez Cappa, S. et al., Am. J. Trop. Med. Hyg., 36:41-45 (1987); Losavio, A., et al., Am. J. Trop. Med. Hyg., 41:539-547 (1989)). The disease can progress to a lethal chronic stage. In this stage, patients exhibit autonomic neuropathy characterized by extensive fibrotic and inflammatory lesions in the muscles, autonomic ganglia, and nerves of the heart and GI tract (Köberle, F., Adv. Parasitol., 6:63-116 (1968); Andrade, Z., Ciba Found. Symp., 99:214-233 (1983); Adad, S. et al., Rev. Inst. Med. Trop. Sao Paulo, 33:443-450 (1991)). The molecular mechanisms that account for the long-lasting absence of neuropathy in the indeterminate phase and for the neurological manifestations in the chronic disease are unknown.

As described herein, a study of the effects of T. cruzi extracts and proteins on neurons and glial cells (e.g., Schwann cells) was conducted. In the course of this study it was determined that the T. cruzi neuraminadase (also referred to as trans-sialidase (TS)) can induce neurite outgrowth in neuronal cell lines, and can inhibit (i.e., reduce or prevent) neurotrophic factor withdrawal-induced apoptotic death of neuronal cells, glial cells and primary neurons. In fact, TS was more effective on a molar base than the prototypic mammalian neurotrophic factor NGF in preventing apoptosis of neuronal cells and primary neurons.

In further studies, the regions of TS which confer neurotrophic activity on the protein were identified. It was determined that TS and peptides derived from the catalytic domain of TS can directly induce neurite outgrowth and can protect neurons from neurotrophic factor withdrawal-induced apoptosis. In addition, it was determined that TS and peptides derived from the tandem-repeat domain of TS can induce the secretion of IL-6, a recognized neurotrophic factor that can protect neurons from apoptosis. Thus, TS can promote the differentiation and inhibit apoptosis of neurons directly and/or indirectly. For example, TS can induce production of endogenous neurotrophic factors (e.g., IL-6) and/or provide trophic support for glial cells (e.g., Schwann cells) which in turn support neurons.

The invention relates to TS and to the neurotrophic and IL-6 secretion-inducing activities of the protein. In one aspect, the invention relates to a method of providing trophic support for neurons and/or glial cells (e.g., Schwann cells) in a mammal (e.g., a human), comprising administering to the mammal a therapeutically effective amount of TS (e.g., SEQ ID NO:2, SEQ ID NO:34) or a neurotrophic variant thereof. The TS can be a naturally occurring enzyme which has neurotrophic and/or IL-6 secretion-inducing activity, an active variant thereof or an active fragment of a naturally occurring enzyme or active variant thereof. As used herein, “active variant” refers to variant proteins and/or peptides which have neurotrophic and/or IL-6 secretion-inducing activity. An “active variant” does not have to have neuraminadase or trans-sialidase catalytic activity. An active variant TS differs in amino acid sequence from a reference TS, such as the TS encoded by clone 19Y deposited in GenBank under accession number AJ002174 (SEQ ID NO:2) or the TS encoded by clone 7F deposited in GenBank under accession number M61732 (SEQ ID NO:34), but retains neurotrophic and/or IL-6 secretion-inducing activity. Generally, differences are limited so that the sequences of the reference polypeptide and the active variant are closely similar overall and, in many regions, identical. An active variant TS and a reference TS can differ in amino acid sequence by one or more amino acid substitutions, additions, deletions, truncations, fusions or any combination thereof. Preferably, amino acid substitutions are conservative substitutions. A conservative amino acid substitution refers to the replacement of a first amino acid by a second amino acid that has chemical and/or physical properties (e.g., charge, structure, polarity, hydrophobicity/hydrophilicity) which are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Active variant TSs include naturally occurring variants (e.g., allelic forms) and variants which are not known to occur naturally. As used herein, the term “active variant” includes fusion proteins.

Fusion proteins encompass polypeptides comprising TS (e.g., SEQ ID NO:2, SEQ ID NO:34) or an active variant thereof as a first moiety, linked via a covalent bond (e.g., a peptide bond) to a second moiety (a fusion partner) not occurring in the TS as found in nature. Thus, the second moiety can be an amino acid, oligopeptide or polypeptide. The second moiety can be linked to the first moiety at a suitable position, for example, the N-terminus, the C-terminus or internally. In one embodiment, the fusion protein comprises an affinity ligand (e.g., an enzyme, an antigen, epitope tag, a binding domain) and a linker sequence as the second moiety, and TS or a active portion thereof as the first moiety. Additional (e.g., third, fourth) moieties can be present as appropriate. The second (and additional moieties) can be any amino acid, oligopeptide or polypeptide that does not interfere with the neurotrophic or IL-6 secretion-inducing activity of TS.

Active variants of TS can be prepared using suitable methods, for example, by direct synthesis, mutagenesis (e.g., site directed mutagenesis, scanning mutagenesis) and other methods of recombinant DNA technology. Active variants can be identified and/or selected using a suitable assay, such as the neurite outgrowth, apoptosis and IL-6 assays described herein.

In one embodiment, an active variant of TS (e.g., neurotrophic variant, IL-6 secretion inducing variant) shares at least about 80% amino acid sequence similarity or identity with a naturally occurring TS (e.g., SEQ ID NO:2, SEQ ID NO:34), preferably at least about 90% amino acid sequence similarity or identity, and more preferably at least about 95% amino acid sequence similarity or identity with said TS. In another embodiment, a fusion protein comprises a first moiety which shares at least about 85% sequence similarity or identity with a TS (e.g., SEQ ID NO:2, SEQ ID NO:34), preferably at least about 90% sequence similarity or identity, and more preferably at least about 95% sequence similarity or identity with a TS. In another embodiment, the active variant comprises fewer amino acid residues than a naturally occurring TS. In this situation, the variant can share at least about 80% amino acid sequence similarity or identity with a corresponding portion of a naturally occurring TS (e.g., catalytic domain (amino acid residues 33-666 of SEQ ID NO:2, amino acid residues 33-666 of SEQ ID NO:34)), preferably at least about 90% amino acid sequence similarity or identity, and more preferably at least about 95% amino acid sequence similarity or identity with a corresponding portion of said TS. Portions of the amino acid sequence of TS which correspond to a variant and amino acid sequence similarity or identity can be identified using a suitable sequence alignment algorithm, such as ‘BLAST 2 Sequences’ using default parameters (Tatusova, T. et al., FEMS Microbiol. Lett., 174:187-188 (1999)).

In one embodiment, the active variant comprises the amino acid sequence of peptide C44 (SEQ ID NO:12). In another embodiment, the active variant comprises the amino acid sequence of peptide C14 (SEQ ID NO:14).

In a preferred embodiment, TS or a neurotrophic variant thereof is co-administered to the mammal with a synergistic amount of a mammalian neurotrophic factor, such as a factor selected from the IL-6 family (e.g., IL-6, IL-11, LIF, CNTF, OSM). As used herein, the term “synergistic amount” refers to a quantity of a mammalian neurotrophic factor which, when co-administered with TS, produces a neurotrophic effect that is greater than the sum of the effects of each agent individually. A synergistic amount can be an amount which provides little or no trophic support when administered without TS. Thus, the amount of each agent that is administered to the mammal can be greatly reduced and undesirable side effects, such as those observed when certain mammalian neurotrophic factors are administered (e.g., CNTF), can be substantially reduced or eliminated. Preferably, the co-administered neurotrophic factor is CNTF or LIF.

In additional embodiments, the active variant is a fusion protein comprising TS or a neurotrophic variant thereof as a first moiety and a suitable fusion partner as a second moiety. In one embodiment, the fusion protein comprises the amino acid sequence of peptide C14 (SEQ ID NO:14) or a neurotrophic variant thereof. In another embodiment, the fusion protein comprises two or more of the 12 amino acid repeats (e.g., peptide TR1 (SEQ ID NO:32)) of the TS tandem repeat domain. In another embodiment, the fusion protein comprises an amino acid sequence in which the amino acid sequence of peptide TR1 (SEQ ID NO:32) or an IL-6 inducing variant thereof occurs at least twice. In another embodiment, the fusion protein comprises a second moiety (fusion partner) which is a mammalian (e.g., human) neurotrophic factor. In a particular embodiment, the fusion protein comprises a second moiety (fusion partner) that is selected from the group consisting of CNTF and LIF.

Under certain circumstances it can be advantageous to administer one or more neurotrophic and/or IL-6 secretion-inducing peptides which are based on the sequence of TS to the mammal. For example, peptides which are directly neurotrophic can be administered, thereby providing neurotrophic support without inducing the expression of IL-6 or other growth factors which can be undesirable in certain circumstances.

Peptides C44 (SEQ ID NO:12), CFN-1 (SEQ ID NO:13) and C14 (SEQ ID NO:14) which are based on the sequence of the TS catalytic domain can be administered to a mammal to provide trophic support for neurons and/or glial cells. It is anticipated that variants of these peptides can retain neurotrophic activity and be suitable for use in the method of the invention. Such active variants can differ in amino acid sequence from the reference peptide by one or more amino acid substitutions, additions, deletions, truncations, fusions or any combination thereof as described herein. It is further anticipated the peptides which comprise fewer than fourteen amino acids can have neurotrophic activity. Such peptides can be prepared by deleting one or more terminal and/or internal amino acids from peptide C14 (SEQ ID NO:14) using conventional methods and assaying the peptides for neurotrophic activity as described. For example, the peptide can have about 3 to about 13 amino acids.

In another embodiment, the invention is a method of providing neurotrophic support for neurons and/or glial cells in a mammal, comprising administering to said mammal a therapeutically effective amount of a peptide comprising the amino acid sequence of peptide C14 (SEQ ID NO:14) or a neurotrophic variant thereof. In another embodiment, the neurotrophic peptide is co-administered with a synergistic amount of a mammalian neurotrophic factor.

The amino and carboxyl termini of the peptides described herein can each, independently, be unprotected or protected by a suitable protecting group. Suitable groups for protecting amino and carboxyl groups are known in the art. See, for example, Greene and Wuts, “Protective Groups in Organic Synthesis,” John Wiley & Sons (1991), the teachings of which are incorporated herein by reference.

The method can be employed to provide trophic support for neurons and/or glial cells in a mammal in need thereof. For example, the method can be employed to treat a mammal with a congenital or acquired condition characterized by neural degeneration, such as amyotrophic lateral sclerosis, Alzheimer's disease, Parkinson's disease, Huntington's disease, Chagas' disease, peripheral neuropathy, palsies (e.g., cerebral, facial, Bell's, bulbar, gaze, oculomotor, progressive supranuclear, trochler), multiple sclerosis and the like. The method can also be employed to treat a mammal which has experienced a stroke (ischemic stroke) or trauma to the brain, spinal cord or peripheral nerves.

TS and peptides which induce the secretion of IL-6 can be administered to a mammal to provide neurotrophic support or for other therapeutic purposes. As described herein, TS and peptides which comprise an amino acid sequence in which the amino acid sequence of peptide TR1 (SEQ ID NO:32) occurs at least twice can induce the secretion of IL-6. Preferably, the amino acid sequence of peptide TR1 (SEQ ID NO:32) occurs about five or about six times in tandem (i.e., one occurrence of the sequence is immediately preceded and/or followed by another occurrence of the sequence). It is anticipated that peptides comprising an amino acid sequence in which the amino acid sequence of a variant of peptide TR1 (SEQ ID NO:32) occurs at least twice can induce the secretion IL-6. Such variants can be prepared using conventional methods and assessed for IL-6 secretion inducing activity as described herein.

In another aspect, the invention is a method of stimulating the secretion of IL-6 in a mammal (e.g., a human), comprising administering to the mammal a therapeutically effective amount of TS or an IL-6 secretion-inducing variant thereof. In one embodiment, the variant comprises an amino acid sequence in which the amino acid sequence of peptide TR1 (SEQ ID NO:32) occurs at least twice. In another embodiment, the variant is a fusion protein. In another embodiment, a peptide comprising an amino acid sequence in which the amino acid sequence of peptide TR1 (SEQ ID NO:32) or an IL-6 secretion-inducing variant thereof occurs at least twice is administered to a mammal to stimulate the secretion of IL-6.

IL-6 is a cytokine with a variety of biological activities including regulation of the immune response, hematopoiesis and inflammation, in addition to neurotrophic activity. Thus, the method of the stimulating the secretion of IL-6 can be used to treat a mammal in need of neurotrophic support or modulation of the immune response, hematopoiesis or inflammatory response.

According to the methods of the invention, TS, active variants thereof (e.g., peptides, fusion proteins) and/or other therapeutic agents (e.g., CNTF, LIF) can be administered to the mammal by any appropriate route. A therapeutically effective amount is administered. A therapeutically effective amount is an amount sufficient to achieve the desired therapeutic or prophylactic effect, under the conditions of administration, such as an amount which is sufficient to induce neural development or regeneration, inhibit (i.e., reduce or prevent) apoptosis of neurons and/or glial cells, improve the neurological functions of the mammal, induce the expression of the bcl-2 gene, activate Akt kinase or induce the secretion of IL-6. TS, active variants thereof (e.g., peptides, fusion proteins) and any other agent (e.g., mammalian neurotrophic factor) to be administered can be administered in a single dose or multiple doses. The dosage can be determined by methods known in the art and is dependent, for example, upon the type of disorder, the mammal's age, sensitivity and tolerance to drugs, and overall well-being. Typically, an effective amount can range from about 0.001 mg/kg per day to about 10 mg/kg per day for an adult.

A variety of routes of administration are possible including, for example, oral, dietary, topical, transdermal, rectal, parenteral (e.g., intravenous, intraarterial, intraperitoneal, intramuscular, intrathecal, intracerebral, subcutaneous injection, intradermal injection), and inhalation (e.g., intrabronchial, intranasal or oral inhalation, intranasal drops) routes of administration, depending on the condition to be treated. Administration can be local or systemic as indicated. The preferred mode of administration can vary depending upon the particular condition (e.g., disease) being treated, however, parenteral or oral administration is generally preferred.

TS and/or active variants thereof (e.g., peptides, fusion proteins) and any additional therapeutic agents can be administered as neutral compounds or as physiologically acceptable salts. Salts of compounds containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

TS and/or active variants thereof (e.g., peptides, fusion proteins) can be administered to the mammal as part of a composition comprising an isolated TS and/or active variant thereof (e.g., peptides, fusion proteins) and a pharmaceutically or physiologically acceptable carrier. The composition can further comprise an additional therapeutic agent, such as a mammalian neurotrophic factor (e.g., CNTF, LIF). Formulation will vary according to the route of administration selected (e.g., solution, emulsion, capsule). Suitable physiological carriers can contain inert ingredients which do not interact with TS, variants, peptides and agents. Standard pharmaceutical formulation techniques can be employed, such as those described in Remington's Pharmaceutical Sciences, Mack Publishing Company, Easton, Pa. Suitable physiological carriers for parenteral administration include, for example, sterile water, physiological saline, bacteriostatic saline (saline containing about 0.9% benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents,” John Wiley and Sons, 1986). For inhalation, the agent can be solubilized and loaded into a suitable dispenser for administration (e.g., an atomizer, nebulizer or pressurized aerosol dispenser).

Furthermore, TS and/or active variants thereof (e.g., peptides, fusion proteins) can be administered via in vivo expression of the recombinant protein. In vivo expression can be accomplished via somatic cell expression according to suitable methods (see, e.g., U.S. Pat. No. 5,399,346). In this embodiment, a nucleic acid encoding the protein or peptide can be incorporated into a retroviral, adenoviral or other suitable vector (preferably, a replication deficient infectious vector) for delivery, or can be introduced into a transfected or transformed host cell capable of expressing the protein or peptide for delivery. In the latter embodiment, the cells can be implanted (alone or in a barrier device), injected or otherwise introduced in an amount effective to express the protein or peptide in a therapeutically effective amount.

In another aspect, the invention relates to the active peptides described herein. In one embodiment, the invention is a neurotrophic peptide comprising the amino acid sequence of peptide C14 (SEQ ID NO:14) or a neurotrophic variant thereof. In another embodiment, the neurotrophic peptide comprises an amino-terminal protecting group, a carboxyl-terminal protecting group or a combination thereof. In another embodiment, the invention is an IL-6 secretion-inducing peptide comprising an amino acid sequence in which the amino acid sequence of peptide TR1 (SEQ ID NO:32) or an IL-6 secretion-inducing variant thereof occurs at least twice. The IL-6 secretion-inducing peptide can further comprise an amino-terminal protecting group, a carboxyl-terminal protecting group or a combination thereof.

In another aspect, the invention relates to the active TS variants described herein. In one embodiment, the invention is a fusion protein wherein the TS component comprises the amino acid sequence of peptide C14 (SEQ ID NO:14) or a neurotrophic variant thereof. In another embodiment, the fusion partner is a mammalian neurotrophic factor, such as CNTF or LIF. In an additional embodiment, the fusion protein comprises an amino acid sequence in which the amino acid sequence of peptide TR1 (SEQ ID NO:32) or an IL-6 secretion-inducing variant thereof occurs at least twice, and a suitable fusion partner.

In another aspect, the invention relates to compositions comprising TS, active variants thereof (e.g., neurotrophic and/or IL-6 secretion-inducing peptides, as described herein), and a physiologically acceptable carrier. The compositions can further comprise a mammalian neurotrophic factor. In one embodiment, the composition comprises a neurotrophic peptide comprising the amino acid sequence of peptide C14 (SEQ ID NO:14) or a neurotrophic variant thereof. In another embodiment, the composition comprises a fusion protein wherein the TS component comprises the amino acid sequence of peptide C14 (SEQ ID NO:14) or a neurotrophic variant thereof. In another embodiment, the composition comprises an IL-6 secretion-inducing peptide comprising an amino acid sequence in which the amino acid sequence of peptide TR1 (SEQ ID NO:32) or an IL-6 secretion-inducing variant thereof occurs at least twice. In another embodiment, the composition comprises an IL-6 secretion-inducing fusion protein comprising an amino acid sequence in which the amino acid sequence of peptide TR1 (SEQ ID NO:32) or an IL-6 secretion-inducing variant thereof occurs at least twice. In a particular embodiment, the composition comprises TS or a neurotrophic variant thereof, a mammalian neurotrophic factor and a physiologically acceptable carrier.

The invention further relates to TS, active variants thereof (e.g., neurotrophic and/or IL-6 secretion-inducing peptides having amino acid sequences based on TS) as described herein for use in therapy (including prophylaxis) or diagnosis, and to the use of TS, active variants thereof and active peptides having amino acid sequences based on TS for the manufacture of a medicament for the treatment of a particular disease or condition as described herein (e.g., amyotrophic lateral sclerosis, Alzheimer's disease, Parkinson's disease, Huntington's disease, Chagas' disease, peripheral neuropathy, palsies (e.g., cerebral, facial, Bell's, bulbar, gaze, oculomotor, progressive supranuclear, trochler), multiple sclerosis, stroke, brain trauma, spinal cord trauma and peripheral nerve trauma).

### EXEMPLIFICATION

### Example 1

### TS and Peptides Derived From TS Promote Survival of Neurons

### Materials and Methods

### Growth Factors, Cytokines, and Synthetic Peptides

Mouse 7S nerve growth factor (NGF) was purchased from Collaborative Biomedical Products (Bedford, Mass.); human ciliary neurotrophic factor (CNTF) was a gift of Dr. E. Granowitz (New England Medical Center, Boston, Mass.); and recombinant human and rat CNTF, as well as the neuraminidases from V. cholera, C. perfringens, and Newcastle disease virus, were from Calbiochem. Recombinant human IL-11, leukemia inhibitory factor (LIF), and oncostatin-M were from Sigma (St. Louis, Mo.), while recombinant human IL-6 was from Endogen. Synthetic peptides were made at the Tufts Biotechnology Center (Tufts University, Boston, Mass.). The peptides were dissolved in RPMI/0.2% BSA prior to the neuronal assays.

### Purification of TS

TS was purified by immuno-affinity chromatography as described previously (Scudder, P. et al., J. Biol. Chem., 268(13): 9886-9891 (1993)). In brief, supernatants from T. cruzi-infected Vero cells were passed on a protein-G Sepharose (Pharmacia) column adsorbed with mAb TCN-2 specific for the LTR domain of TS. Bound TS was eluted with TR1 peptide (SEQ ID NO:32) in the presence of 0.1% octyl glucopyranoside. TS was concentrated by ultrafiltration in Amicon-10 and washed extensively with PBS pH 7.8 to remove the TR1 (SEQ ID NO:32) peptide.

The neuraminidase from Vibrio cholera was purchased from Calbiochem. Penetrin was isolated by heparin-affinity chromatography as described previously (Ortega-Barria, E. et al., Cell, 67: 411-421 (1991)). All reagents, glassware and plasticware used in the isolation of the various proteins were LPS-free. To eliminate residual LPS, the purified materials were passed through two distinct resins that remove endotoxin by different mechanisms, END-X B15 (Associates of Cape Cod, Woods Hole, Mass.) and AffinityPak Detoxi-Gel (Pierce, Rockford, Ill.), following the recommendations of the manufacturers.

### Cloning and Expression of Recombinant Fragments of Trans-Sialidase

The DNA fragments corresponding to the catalytic domain of TS were amplified by PCR using, as templates, TS clone 19Y (SEQ ID NO:1) or clone 7F (SEQ ID NO:33) from a genomic DNA library of T. cruzi trypomastigote clone MV-13 (Pereira, M. et al., J. Exp. Med., 174:179-191 (1991), incorporated herein by reference). A 1899 bp segment of DNA encoding the full catalytic domain of TS, referred to herein as TS-F (also referred to herein as CD), was obtained with synthetic DNA primers TS1 5′-GGAATTCCATATGGCACCCGGATCGAGCCGAGTT-3′ (SEQ ID NO:3) and MP10 5′-CCGCTCGAGGCTCAAGAACAAGGTCCTGATCG-3′ (SEQ ID NO:4). For amplification of DNA encoding fragments TS-F-47, TS-CC-47 and TS-Cat-47 of TS, a common forward synthetic DNA primer Mpl 35′-GGGAATTCGGTTGCCAATCGCGGACGCTC-3′ (SEQ ID NO:5) was used together with reverse DNA primer MP10 (SEQ ID NO:4), MP12 5′-CCCCTCGAGATTTGCCGTGCTTGCGT-3′ (SEQ ID NO:6) or MP11 5′-CCCCTCGAGCCGACAAAAAGCCAACAAAGAC-3′ (SEQ ID NO:7), respectively. DNA encoding fragment TS-F-47 was prepared using clone 7F as template. DNAs encoding fragments TS-CC-47 or TS-Cat-47 were prepared using clone 19Y as template. The amplified DNA fragments were cloned into pET 23b (Novagen) to generate a construct encoding a stretch of 6 histidine residues at the C-terminus of the expressed proteins. A plasmid encoding CD from clone 7F and a plasmid encoding CD from clone 19Y were produced. CD from clone 19Y or from 7F was active in the studies using PC12 cells.

For protein production, plasmids were transferred to E. coli strain B12-1 DE3 (Novagen), containing a chromosomal copy of the T7 RNA polymerase gene. Expression was induced by Isopropyl-β-D-thiogalactopyranoside (IPTG.) To isolate the recombinant TS fragments, bacterial lysates were prepared by osmotic shock in 40 mM phosphate buffer, 0.3 M NaCl, 1% Triton X-100, 1 mM PMSF, followed by brief sonication. In the lysates containing TS-F-47, TS-CC-47 and TS-Cat-47 urea was added to 8 M, to facilitate solubilization. Suspensions were centrifuged for 30 minutes at 20,000×g and the supernatant loaded on a Ni²⁺ (−) nitrilotriacetic acid/agarose column as recommended by the manufacturer (Novagen). For refolding of the urea-soluble proteins the purified polypeptides were incubated in a solution containing 8 M urea, 50 mM Tris/HCl, pH 8.0, 5 mM 1,4-dithio-DL-threitol, and 1 mM EDTA. Reduction was performed overnight at 4° C. The solution was then diluted with 4 vol of 50 mM Tris/HCl, pH 8.0 containing 1.25 mM reduced and oxidized glutathione, followed by dialysis overnight against 10 mM Tris/HCl, pH 8.0 at 4° C. (see, for example, Marti, D. et al., Eur. J. Biochem., 219:455-462 (1994)). Urea was not required for solubilization of TS-F fragment, which was purified by affinity chromatography on Ni²⁺ (−) agarose and by FPLC on the anion-exchange column MonoQ HR (Pharmacia), as previously described (Scudder, P. et al., J. Biol. Chem., 268: 9886-9891 (1993)).

The full-length C-terminal long tandem repeat (LTR fragment) of TS was generated in insect cells. LTR, sucloned from a pMelBac plasmid (Invitrogen) containing the TS gene of clone 7F (Pereira, M. et al., J. Exp. Med., 174:179-191 (1991), incorporated herein by reference), was digested with Pvu II/Sal I and ligated into EcoR V/Sal I sites of pET20b (Novagen). The LTR DNA was introduced into the Nco I/Hind III sites of pFASTBAC HTb vector (Gibco BRL). The Bac- to Bac system (Gibco BRL) was used to generate recombinant baculovirus, which in turn was used to infect Sf9 cells. Recombinant LTR protein was purified by Ni²⁺-NTA column (Novagen), followed by affinity chromatography on mAb TCN-2 (Prioli, R. et al., Mol. Biochem. Parasitol., 52:85-96 (1992)). The LTR fragment thus generated contains the full-length tandem repeat of clone 7F plus a TS sequence of 26 amino acids upstream and 40 amino acids downstream of the repeat.

The relative amount of recombinant protein produced by the bacteria was determined by quantifying Pro Blue (Integration Separation System) stained proteins separated by 10% SDS-PAGE using a Gel Doc 1000 apparatus (Bio Rad). Various quantities of BSA were run on the gels as standards. Alternatively, the recombinant proteins were blotted to nitrocellulose membrane and visualised with T7-Tag antibody (Novagen) or with a mouse polyclonal antibody against TS.

### Cell Cultures

PC12 cells were obtained from ATCC (Manassas, Va. (Accession No. CRL-1721)) and cultured on collagen-coated dishes in RPMI 1640 supplemented with 10% horse serum and 5% fetal bovine serum (Greene, L., and Tishler, A., Proc. Natl. Acad. Sci. USA, 73:2424-2428 (1976)). For differentiation and survival experiments, cells were washed three times in serum-free RPMI/0.2% BSA, plated in the same medium in collagen-coated plastic dishes at 2×10⁵/ml, without and with growth factors, TS, recombinant proteins, synthetic peptides, for the time and concentrations indicated in the Figures.

### Neurite Outgrowth Assay

96-well microtiter plates were coated overnight at 4° C. with the TS or peptides or fragments thereof, or with control proteins laminin (Ln) or bovine serum albumin (BSA) at 500 μg/ml. After removing the compounds, the plates were further incubated with 1% BSA for 1 hour at room temperature and immediately used as substratum for PC12 or N18 (Prasad, K., Biol. Rev. Camb. Philos. Soc., 66:431-451 (1991)) cells in serum-free RPMI/0.2% BSA. Neurite outgrowth was measured using a phase-contrast microscope 17 hours later. Cells exhibiting neurite outgrowth were those having one or more cytoplasmic extension >2 μm in length.

### Assays for Cell Survival

Cell staining with 4,6-diamino-2-phenylindole (DAPI). Cells were fixed with 4% formaldehyde in PBS for 5 minutes, washed with PBS, stained with 10 μg/ml DAPI (Sigma) for 2 minutes, washed with PBS, and visualized under UV light using a fluorescent microscope. About 300-400 cells were examined under the microscope to determine the percentage of apoptotic cells.

Cell DNA nick end labeling (TUNEL) (Gavrieli, Y. et al., J. Cell. Biol., 119:493-501 (1992)) assay was performed using a kit purchased from Boehringer Mannheim as described by the manufacturer.

Protection against apoptosis of PC12 cells in serum-free medium containing a test agent (agent N) (e.g., protection induced by TS, TS fragments, NGF or other growth factors), was calculated by the formula: 100%−[(apoptosis(%) of cells kept in RPMI containing test agent N÷apoptosis (%) of cells kept in RPMI)×100%]. Testing the effect of Wortmannin (Sigma) and LY294002 (Sigma) in inhibiting the neuroprotection of TS was performed following the protocol described by others (Yao, R., and Cooper, G., Science, 267:2003-2006 (1995); D'Mello, et al., J. Neurosci., 17:1548-1560 (1997)). In brief, PC12 cells were switched from growth medium to RPMI without and with TS or various other growth factors. After 24 hours, Wortmannin and LY294002 were added to the cultures. After another 24 hours, cells were examined for apoptosis by the DAPI assay, as above.

### RNA Preparation and Reverse Transcription Reactions.

Total RNA was extracted from PC12 cells by the acid guanidinum isothiocyanate method (Chomchinski, P., Biotechniques, 15:532-535 (1993)) using TRI-reagent (Molecular Research Center, Inc.) cDNA synthesis was performed according to the instruction of the manufacturer (Gibco-BRL). Reverse transcription reactions were carried out for 50 minutes at 42° C. and were heated to 70° C. for 15 minutes to terminate the reaction using a MiniCycler (MJ Research). Samples were cooled to 4° C. and stored at −20° C. until use.

### Quantitative PCR of Bcl-2 Gene Transcripts

PCR reactions were performed in 50 μl containing 1.5 μl of template DNA (corresponding to cDNA synthesized from 100 ng of total RNA), 1×PCR buffer, 100 μM of deoxynucleotides, 2.5 mM MgCl₂, 10 mM DTT, 10 μM of Bcl-2 primers, and 2 U of Taq DNA polymerase (Gibco-BRL). The synthetic DNA primers for the rat Bcl-2 were: 5′-AGATGAAGACTCCGCGCCCCTGAGG-3′ (SEQ ID NO:8) and 5′-CCAGGTATGCACCCAGAGTGATG-3′ (SEQ ID NO:9) to give a PCR product of 566 bp. The sequences for synthetic GAPDH DNA primers (Wong, H. et al., Anal. Biochem., 223: 251-258 (1994)) were: 5′-CGGAGTCAACGGATTTGGTCGTAT-3′ (SEQ ID NO:10) and 5′-AGCCTTCTCCATGGTGGTGAAGAC-3′ (SEQ ID NO:11) giving a PCR produce of 306 base pairs. Amplifications were carried out in MiniCycler (MJ Research) using following conditions: 98° C. for 5 minutes (initial heat denaturation), 3 cycles of 94° C. for 1 minute, 63° C. for 1 minute, 72° C. for 1.5 minutes; 3 cycles of 94° C. for 1 minute, 60° C. for 1 minute, 72° C. for 1.5 minutes, followed by 72° C. for 10 minutes. Primers for GAPDH (10 pM) were added at cycle 7 by the “primer-dropping” method. Aliquots of PCR reaction products were separated by electrophoresis in 2% agarose gels (Ultrapure, Gibco-BRL), containing 0.2 μg/ml ethidium bromide. Gels were analyzed by computerized densitometric scanning using Gel doc 1000 (Bio Rad) and Molecular Analyst Software, version 2.1.

### Trans-Sialidase Assay

The trans-sialidase assay was performed as described (Scudder, P. et al., J. Biol. Chem., 268:9886-9891 (1993)) with slight modifications. Various amounts of intact enzyme or enzyme fragments were added to 50 mM PBS, Ph 7.2, containing 15% fetal calf serum and 0.25 mmol of [¹⁴C]N-acetyllactosamine (4×10⁵ dpm), 50 μg BSA and 0.02% NaN₃ in a volume of 60 μl. After incubation for a predetermined amount of time at room temperature, the reaction mixture was diluted to 1.0 ml with distilled water and applied to a column containing 1.0 ml Q-Sepharose equilibrated with water. Sialylated product was eluted with 1 M NaCl and quantified by scintillation counting.

### Primary Neuronal Cultures

Cultures enriched in granule neurons were obtained from the cerebellum of 8-days-old Wistar rats (Charles River, Wilmington, Mass.) as described by D'Mello, S. et al., J. Neurosci., 17: 1548-1560 (1997). Cells were plated in basal Eagle medium (BME, Gibco BRL, Gaithersburg, Md.), supplemented with 10% FCS, 25 mM KCl and 2 mM glutamine (Gibso BRL) on dishes coated with poly-L-lysine and laminin. Cytosine arabinofurnoside (10 μM) was added to the culture medium 18-22 hours after plating to prevent replication of non-neuronal cells. Replacement of culture medium with serum-free BME medium was performed 7-10 days after plating. Cells were maintained in serum-free BME with 5 mM KCl for 3 hours and challenged with different stimuli. Neuronal survival was assayed after 24 hours by staining with 10 μg/ml fluorescein diacetate (FDA, Sigma) (Jones and Senft, J. Histochem. Cytochem. 33:77-9 (1985)) and with DAPI (see above). Chicken dorsal root ganglia neurons were prepared as described (Cox, D. and Dunlap, K., J. Gen. Physiol., 104:311-336 (1994)).

### Immunodetection of Activated Akt

PC12 cells were deprived of serum for 24-48 hours, stimulated with TS, TS-F or LTR for 2, 5 or 10 minutes and immediately lysed with 2% SDS. In studies using the PI-3 kinase inhibitor, LY294002 (LY), cells were pre-treated with the inhibitor (1 μM) for 30 min prior to the addition of TS-F or TS. The proteins in the cell lysate were separated in SDS-10% polyacrylamide gels, transferred to nitrocellulose membrane (Bio-Rad) and the phosphorylated form of protein kinase Akt was detected with phospho-Akt (Ser 473) antibody (New England BioLabs, Beverly, Mass.), followed by alkaline phosphatase-conjugated secondary antibody (Promega, Madison, Wis.). Bands corresponding to phospho-Akt (60 kDa) were quantified using a scanning densitometer (Bio-Rad).

### Results

### TS Promotes Neurite Extension in PC12 and N18 Neuronal Cells

Undifferentiated PC12 and N18 cells extended multiple neurites when live T. cruzi trypomastigotes were added to the liquid overlay of the cultured cells. In addition, conditioned medium prepared by incubating live trypomastigotes in RPMI at 4° C. for 24 hours, also induced neurite outgrowth in both PC12 and N18 cells. Trypomastigote is a mobile, invasive form of T. cruzi that shuttles from the heart, GI tract, and other organs to the circulation, and from the circulation back to peripheral tissues and internal organs. Therefore, trypomastigotes and trypomastigote-secretory products can associate with, and modify the properties of, various cells and proteins throughout the body, including, for example, neurons and neurotrophic factors.

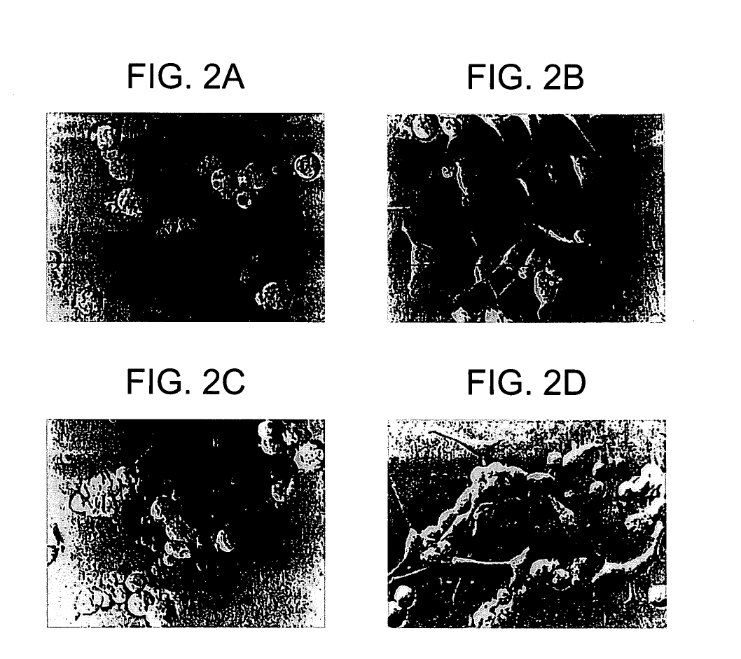

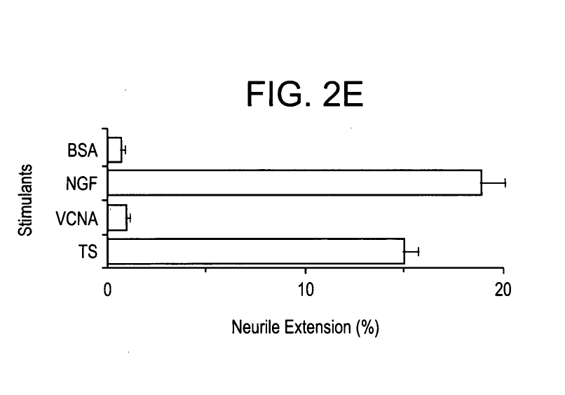

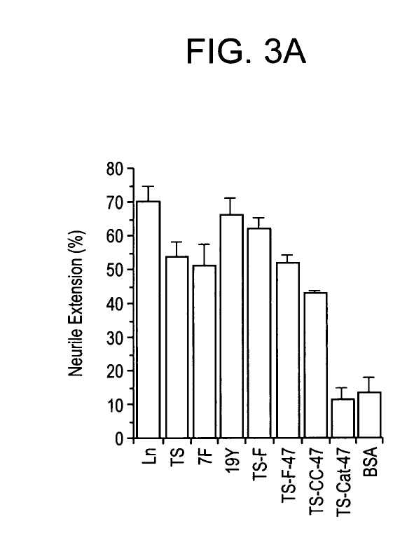

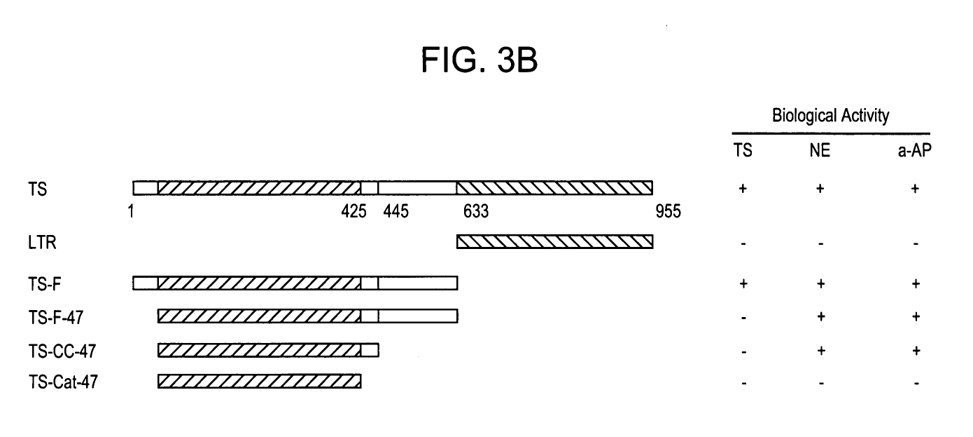

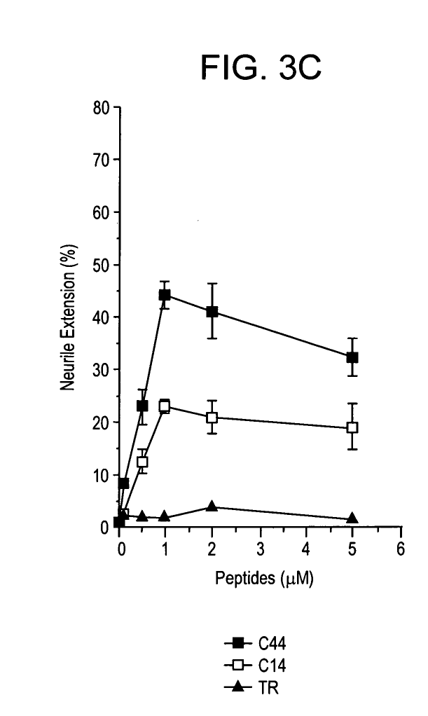

The trypomastigote-conditioned medium comprises TS, proteases such as cruzipain (Murta, A. C. et al., Mol. Biochem. Parasitol., 43:27-38 (1990)), the adhesion molecule penetrin (Pereira, M. et al., J. Exp. Med., 174:179-191 (1991)), and other factors thought to mediate T. cruzi infection (Pereira, M., in Russell, D. Ed., Bailliè re's Clinical Infectious Diseases, pp. 305-334, Bailliè re Tindall, London, (1994)). To identify the neurotrophic factor(s) present in the trypomastigote extract, purified preparations of proteins which are reported to mediate T. cruzi infection (e.g., TS, cruizipain, penetrin) were tested in the neurite outgrown assay. Of the proteins tested, only TS induced neurite outgrowth of both PC12 and N18 neuronal cells. TS induced neurite extension either as a soluble factor in the liquid overlay of the neuronal cell monolayers (FIGS. 2D and 2E) or as a ligand immobilized on the plastic substratum that served as support for cell attachment (FIG. 3A). In solution, TS was active at very low concentrations, namely, in the range of 0.125 nM (25 ng/ml to 200 ng/ml). PC12 neurites became visible 3 hours after a single dose of TS, and remained extended for about 24 hours.

The neurotrophic action of TS was specific for the T. cruzi enzyme as cruzipain and penetrin did not induce neurite extension. Additionally, neuraminidases (sialidases) from other microbes, namely from the bacteria Vibrio cholera (VCNA), Clostridium perfringens and from the Newcastle disease virus did not induce neurite outgrowth (FIGS. 2C and 2E).

TS Protects PC12 Cells from Death Caused by Trophic Factor Deprivation and Induces the Expression of Bcl-2

Neurotrophic factors such as NGF and CNTF regulate the differentiation and maintenance of the nervous system, and are critical to the survival of neuronal cells. Depriving neuronal cells of neurotrophic factors results in the induction of programmed cell death or apoptosis (Deshmukh, M., and Johnson, E., Mol. Pharmacol., 51:897-906 (1997); Pettman, B., and Henderson, C., Neuron, 20:633-647 (1998)). PC12 cells maintain a neuronal phenotype when differentiated in the presence of NGF but undergo apoptosis if deprived of NGF (Rukenstein, A. et al., J. Neurosci., 11:2552-2563 (1991); Mesner, P. et al., J. Cell. Biol., 119:1669-1680 (1992)).

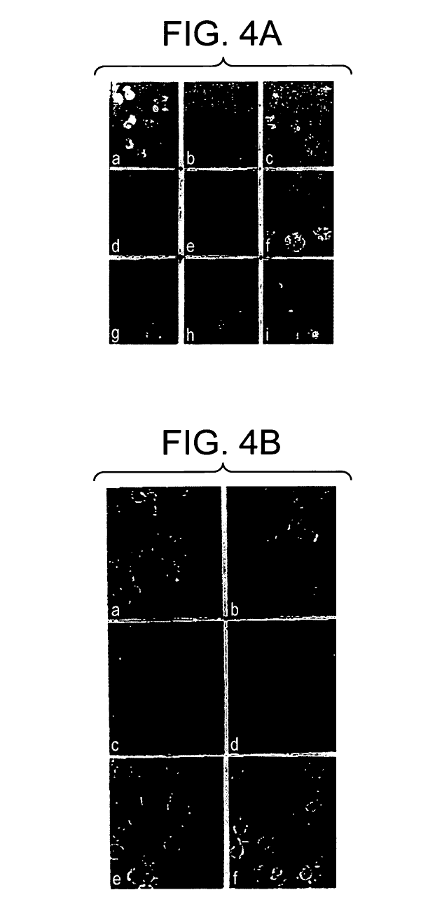

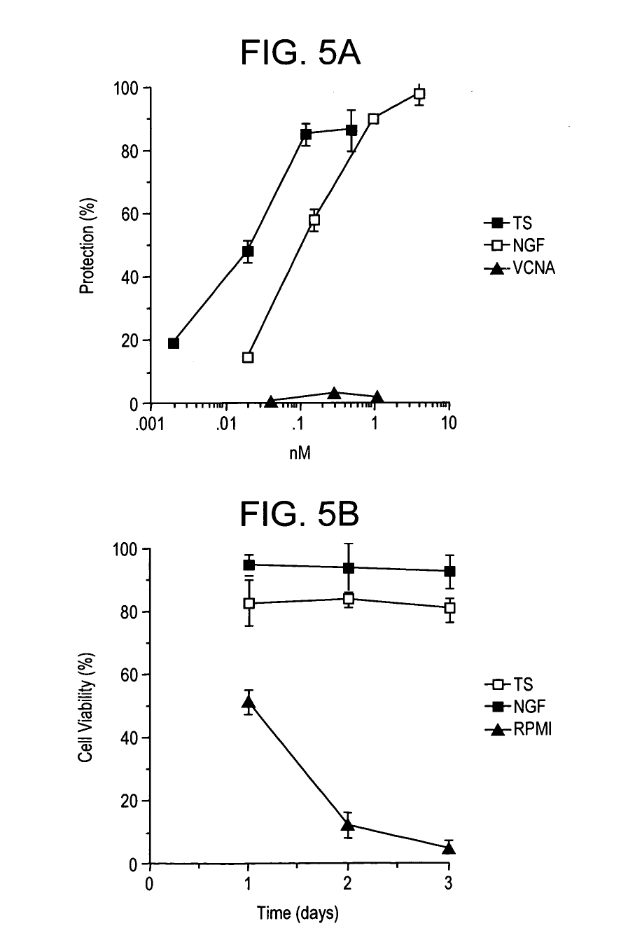

PC12 cells were culture in serum-free medium without and with various concentrations of TS to determine whether TS can rescue neurons from apoptotic death caused by trophic factor (NGF) deprivation. Apoptosis was assessed by counting the number of cells with internucleosomal DNA fragmentation after staining with DAPI (FIG. 4A) or with antibodies to free 3′-OH termini labeled with modified nucleotides (TUNEL assay (FIG. 4B)). TS effectively protected PC12 cells from apoptosis at concentrations in the low pM range under conditions in which the sialidase from V. cholera did not (FIGS. 4A, 4B and 5A). A concentration of TS as low as 62 pM (equivalent to 12.5 ng/ml) protected 45% of PC12 cells from death (FIG. 5A). Protection by a single dose of TS lasted up to several days (FIG. 5B).

### TS Induces Bcl-2 Gene Expression in PC12 Cells

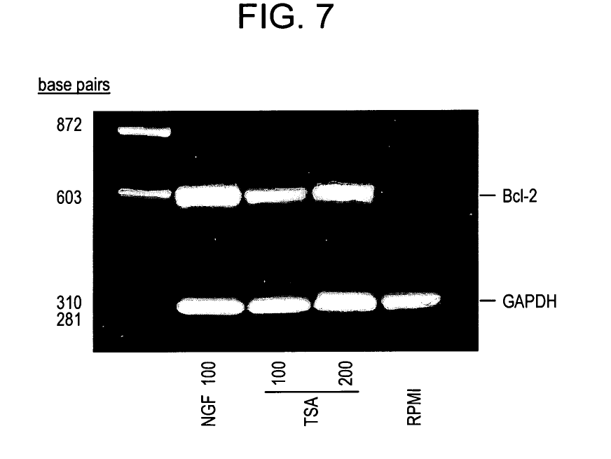

The members of the Bcl-2 family are key regulators of apoptosis in many types of mammalian cells, including neurons (Merry, D. and Korsmeyer, S., Ann. Rev. Neurosci., 20:245-267 (1997)). NGF promotes survival of PC12 cells by inducing overexpression of the anti-apoptotic Bcl-2 gene (Mah, S. et al., J. Neurochem., 60:1183-1186 (1993)). To determine whether TS promotes neuron survival through a similar mechanism, Bcl-2 transcripts were quantified by RT-PCR in PC12 cells grown in serum-free medium with or without TS. TS at low concentrations dramatically increased Bcl-2 mRNA in the neuronal cells starved of mammalian neurotrophic factors, similar to the effect of NGF (FIG. 7). While not being bound by any particular theory, the data presented herein indicate that TS can protect PC12 cells from apoptosis by inducing the expression of the Bcl-2.

### Identification of a TS Epitope That Induces Neurite Extension

The TS of trypomastigotes comprises a Cys-rich catalytic domain of 666 amino acids in the N-terminus and a long 12-amino acid tandem repeat domain in the C-terminus (Pereira, M. et al., J. Exp. Med., 174:179-191 (1991)). To identify a region of the TS molecule that underlies neurite outgrowth, various nucleic acid constructs encoding poly-His-tagged fragments of TS (FIG. 3B) were generated by PCR. The constructs were expressed in E. coli, and the recombinant fragments of TS were purified by Ni²⁺ chelate chromatography (Ni²⁺-agarose) and tested in the neurite outgrowth assay. Fragment LTR, which corresponds to the C-terminal tandem repeat, did not induce neurite outgrowth in PC12 cells nor in neuroblastoma N18 cells (FIG. 3B). In contrast, enzymatically active fragment TS-F, which represents the full-length catalytic domain of TS, induced neurite outgrowth to about the same extent as the intact enzyme. However, the enzymatic activity of TS was not essential for neurite extension. Fragment TS-F-47, generated by deleting 47 amino acids from the N-terminus of fragment TS-F, was enzymatically inactive and yet as effective as the native enzyme in stimulating neurite outgrowth (FIGS. 3A and 3B). Also, the sequence of 188 amino acids at the C-terminus of the catalytic domain was not required for neurotrophic action, as deletion of this sequence from TS-F-47, generating fragment TS-CC-47, did not substantially reduce neurite outgrowth (FIGS. 3A and 3B).

In contrast, deletion of 21 amino acids from the C-terminus of TS-CC-47 produced fragment TS-Cat-47, which was inactive in stimulating neurite outgrowth (FIGS. 3A and 3B). Thus, the region of TS which consists of the 21 amino acid sequence that distinguishes TS-CC-47 from TS-Cat-47 confers neurotrophic activity upon the protein.

In further studies, the synthetic peptides presented in Table 1 were employed. Synthetic peptides CFN1 (SEQ ID NO:13) which comprises the C-terminal 21 amino acids of TS-CC-47, peptide C44 (SEQ ID NO:12) and the fourteen amino acid peptide C14 (SEQ ID NO:14) which are based on the sequence of the catalytic domain of TS had direct neurotrophic activity. These studies confirmed that carboxyl region of the catalytic domain is critical for neuotrophic activity (FIG. 3C).

Synthetic peptides based on other regions of TS, such as peptide TR (SEQ ID NO:19), derived from the tandem repeat (FIG. 3B), and peptide B2 (SEQ ID NO:18), based on a sequence upstream of peptide CFN-1 (FIGS. 3B and 3C), did not induce neurite outgrowth.

TABLE 1
Synthetic Peptides Modeled on the TS Sequence.

| | |
|---|---|
| peptide | amino acid sequence |

| | |
|---|---|
| C44 | QPLRRQRVVVVPLSPRLVLLAFCRQRLPLKRMGGSYRCVNAS |
| | TAN |
| | (SEQ ID NO:12) |
| CFN1 | ⁴²⁵RQRLPKRMGGSYRCVNASTAH⁴⁴⁵ |
| | (SEQ ID NO:13) |
| C14 | RQRLPKRMGGSYRC |
| | (SEQ ID NO:14) |
| C19Y21 | GNASQNVWEDAYRCVNASTAN |
| | (SEQ ID NO:15) |
| CYN2 | ⁴²⁵GNASQNYWEDAYRC⁴³⁸ |
| | (SEQ ID NO:16) |
| CYFN | ⁴³⁹VNASTAN⁴⁴⁵ |
| | (SEQ ID NO:17) |
| B2 | YSVDDGETWE |
| | (SEQ ID NO:18) |
| TR | DSSAHGTPSTPA |
| | (SEQ ID NO:19) |

Peptides C19Y21 and CYN2 are derived from the amino acid sequence of the protein encoded by clone 19Y (GenBank accession number AJ002174). Peptides C44, CFN1, C14 and TR are derived from the amino acid sequence of the protein encoded by clone 7F (GenBank accession number M61732). Peptides CYFN and B2 have amino acid sequences that are common to the proteins encoded by clone 19Y and 7F.

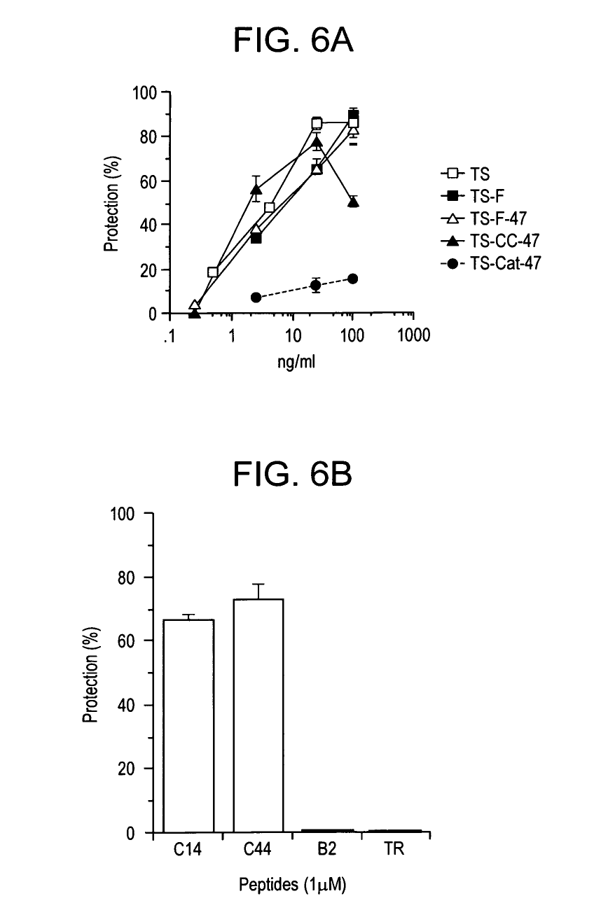

TS-derived recombinant fragments rescued PC12 cells from apoptotic death in a pattern similar to their neurite extension profile. Thus, fragments TS-F-47 and TS-CC-47 were active both in stimulating neurite outgrowth (FIG. 3A) and in preventing apoptosis of PC12 cells (FIG. 6A), whereas fragment TS-Cat-47 did not promote neurite outgrowth (FIG. 3C) as well as it promoted the survival of the PC12 cells (FIG. 6B). Control peptides B2 (SEQ ID NO:18) and TR (SEQ ID NO:19), did not promote neurite outgrowth (FIGS. 3B and 3C) nor survival of the PC12 cells (FIG. 6B).

TS Synergizes with CNTF or LIF to Promote Survival of PC12 Cells

PC12 cells were cultured in serum-free medium without and with TS, alone or in combination with conventional neurotrophic factors, all at concentrations that produce modest or no neuroprotective response. TS was initially tested in combination with the neurotrophins NGF, brain-derived neurotrophic factor (BDNF) and NT-3 (Ip, N.Y., and Yancopoulos, G., Annu. Rev. Neurosci., 19:491-515 (1996)). Such co-administration did not substantially increase neuron survival beyond the anticipated additive-effect of individual neurotrophic factors. For example, TS at 2.5 ng/ml and NGF at 0.5 ng/ml protected 15% and 18% PC12 cells from apoptotic death, respectively, whereas co-administration of TS and NGF at the same concentrations protected 35% of the PC12 cells. Such additive responses were observed when other concentrations of TS (5, 11.5 and 30 ng/ml) were co-administered with NGF (0.5 ng/ml), BDNF (2.0 ng/ml) or NT-3 (2 ng/ml).

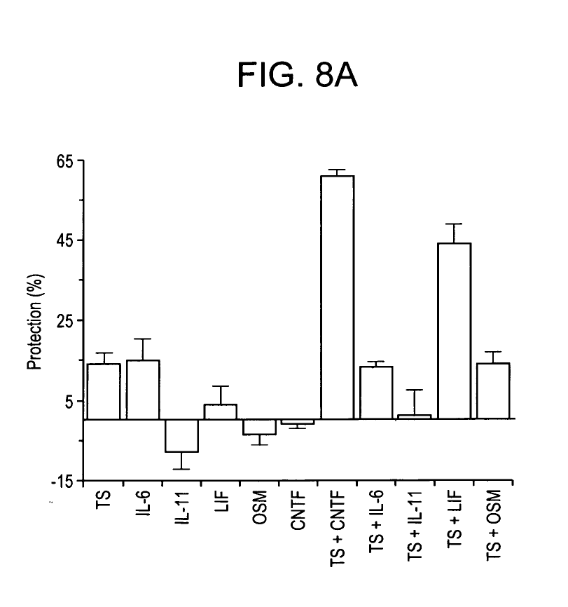

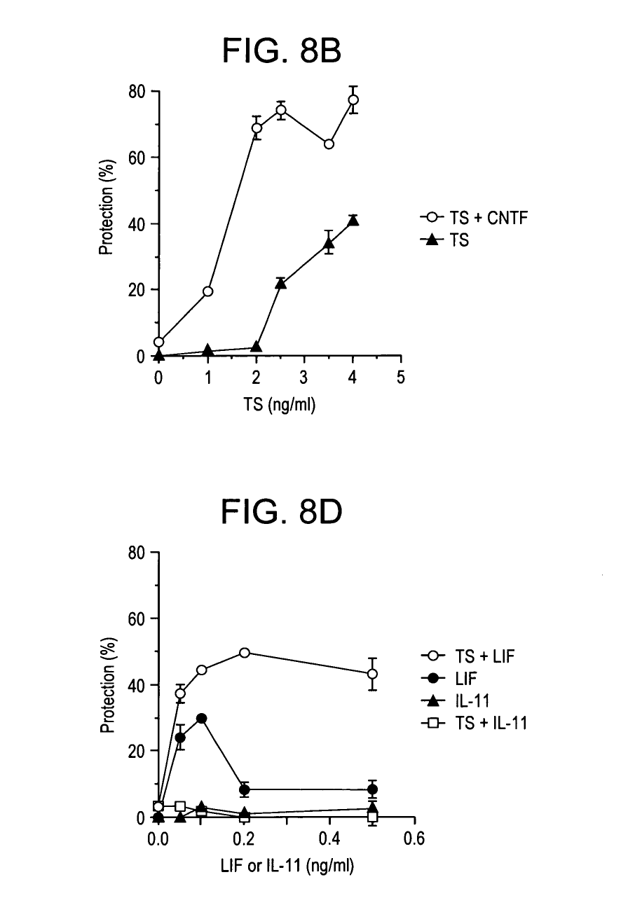

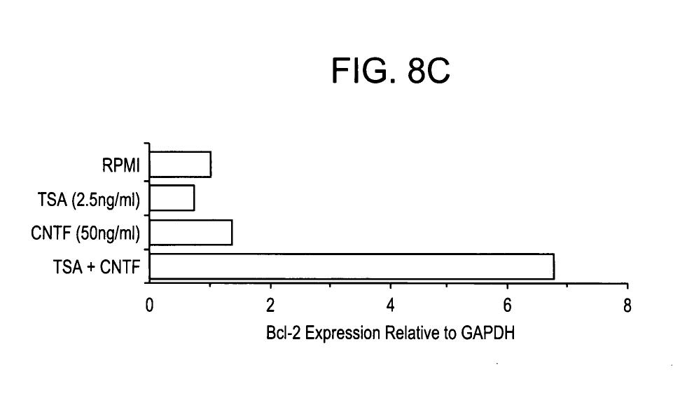

The response of PC12 cells to the combination of TS with neurotrophic factors of the IL-6 family, namely IL-6, IL-11, CNTF, leukemia inhibitory factor (LIF), and oncostatin-M (OSM) (Ip, N.Y., and Yancopoulos, G., Annu. Rev. Neurosci., 19:492-515 (1996)) was also assessed. While TS at 2.5 ng/ml promoted survival in 13% of PC12 cells grown in serum-free medium, co-administration of TS with a subthreshold concentration of CNTF (50 ng/ml) or LIF (0.5 ng/ml) dramatically increased neuron survival to 61% and 45%, respectively (FIG. 8A). Human CNTF and recombinant human and rat CNTF were equally effective in potentiating the TS action of PC12 cells. TS/CNTF and TS/LIF synergism was also observed in the neurite outgrowth assay. Dose-response experiments revealed that the synergism of TS with CNTF or LIF in promoting neuron servival was most remarkable at subthreshold or threshold concentrations of TS (FIGS. 8B and 8D). Confirmation of the synergism between the trypanosomal protein and CNTF was provided by analysis of expression of the Bcl-2 gene in PC12 cells. Bcl-2 transcripts increased by ˜3-4-fold in the PC12 cells treated with the combination of TS and CNTF, relative to the Bcl-2 transcript in the cells treated with only one of the proteins at the same concentration (FIG. 8C). These results demonstrate that TS can promote survival of PC12 cells by upregulating Bc1-2 expression.

### TS Inhibits Serum Withdrawal-Induced Apoptosis of Schwann Cells

Immortalized Schwann cells (Rambukkana, et al., Science, 282:2076-2079 (1998)) were cultured in DMEM supplemented with 10% fetal calf serum (DMEM/10% FCS). For the apoptosis assay, the cells were plated on 16 well slides in DMEM/10% FCS and cultured overnight. The cells were then washed three times with serum-free DMEM and cultured in serum-free DMEM or in serum-free DMEM supplemented with TS (final concentration 0.5 μg/mL, 1 μg/mL, 2 μg/mL or 3 μg/mL). After culture for 24, 48 or 72 hours, the cells were fixed in 4% formaldehyde in PBS for 10 minutes, washed with PBS and stained with DAPI (5 mg/mL). The cells were then washed with PBS and cells with fragmented (apoptotic) nuclei were quantified by visualization under UV light using a fluorescence microscope. 300-400 cells were examined to determine the percentage of apoptotic cells.

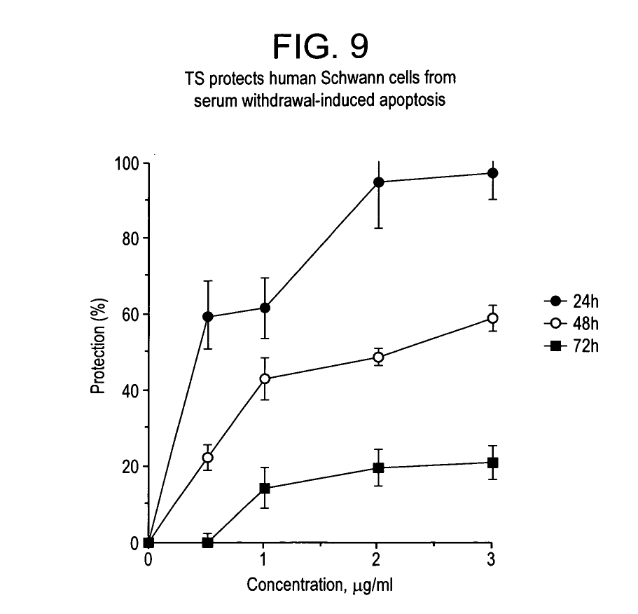

TS effectively inhibited serum withdrawal-induced apoptosis of Schwann cells in the assay (FIG. 9).

### TS Protects Primary Rat Cerebellar Granule Neurons From Apoptosis

Cultured primary cerebellar granule neurons die of apoptosis when switched from a medium containing an elevated concentration of K⁺ (25 mM) to a medium containing a lower K⁺ level (5 mM) (D'Mello, S. et al., Proc. Natl. Acad. Sci. USA, 90:10989-10993 (1993)). Death caused by such potassium depolarization can be prevented by several growth factors such as NGF and insulin-like growth factor (IGF-1) (D'Mello, S. et al., Proc. Natl. Acad. Sci. USA, 90:10989-10993 (1993)).

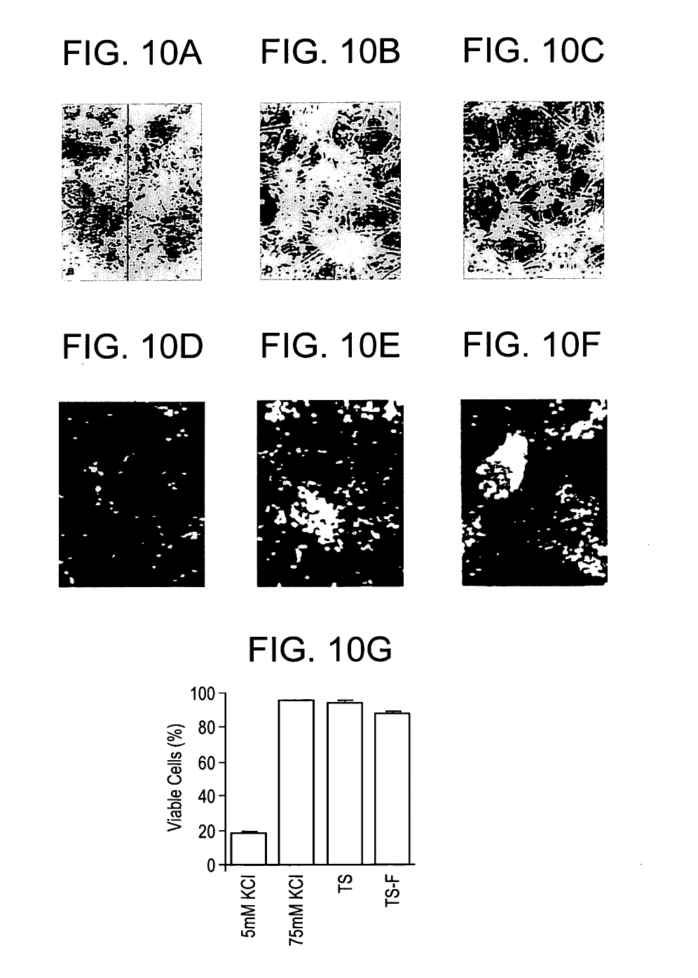

The neuroprotective effects of TS were assessed in cultures of primary cerebellar granule neurons in apoptosis-causing low K⁺ medium supplemented with TS. Neuron viability was ascertained by phase-contrast microscopy (FIGS. 10A-10C) and fluorescent microscopy after staining viable cells with fluorescein diacetate (FIGS. 10D-10F) (Jones and Senft, J. Histochem. Cytochem., 33:77-79 (1985); D'Mello, S. et al., J. Neurosci., 17:1548-1560 (1997)). In agreement with established results (D'Mello, S. et al., J. Neurosci., 17:1548-1560 (1997)), survival of primary cerebellar granule neurons in 5 mM KCl medium (FIGS. 10A, 10D and 10G) was poor relative to survival in 25 mM K⁺ (FIGS. 10C, 10F and 10G). Addition of TS to the apoptosis-causing low K⁺ medium effectively protected the neurons from death, demonstrating that TS provides neurotrophic support for primary neurons.

### Reversal of TS-Induced Neuroprotection by Inhibitors of the Phospholinositide-3 Kinase (PI-3 Kinase)

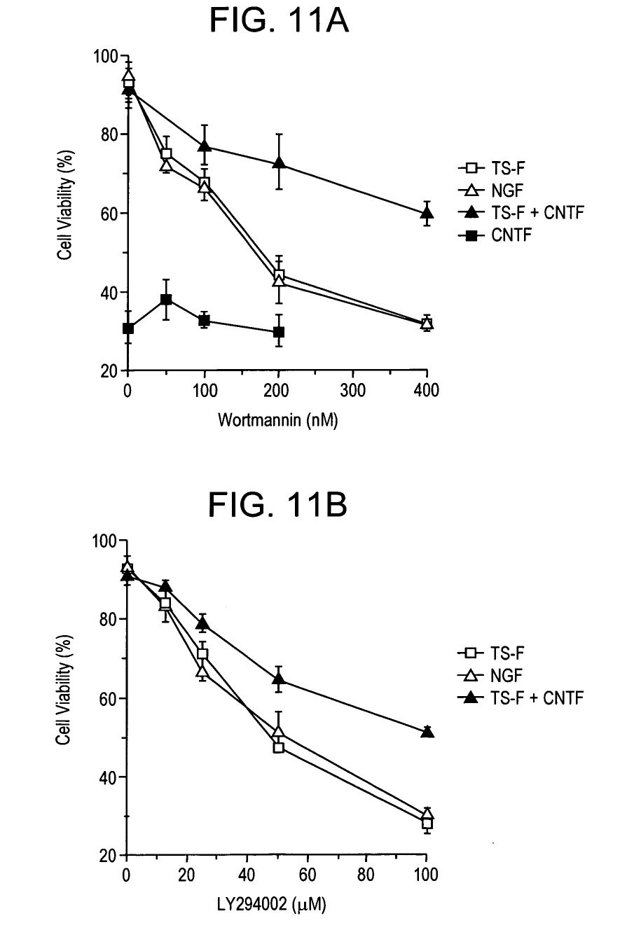

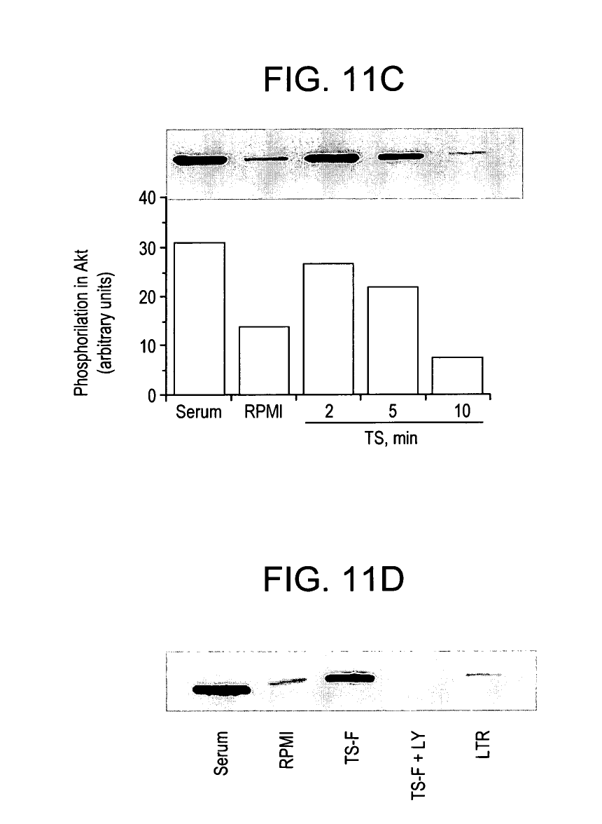

Induction of survival in PC12 cells by NGF, or in cerebellar granule cells by IGF-1 requires signaling through PI-3 kinase, as demonstrated by the use of specific pharmacologic inhibitors (Yao, R. and Cooper, G., Science, 267: 2003-2006 (1995); D'Mello, S. et al., J. Neurosci., 17:1548-1560 (1997)). Wortmannin inhibits PI-3 kinase both in vitro and in vivo (Ui, et al., Trends Pharmacol., 20:303-307 (1995)). Addition of Wortmannin to PC12 cells maintained for 24 hours in serum-free medium supplemented with TS-F, the catalytic domain of TS, reduced neuronal viability in a dose-dependent manner (FIG. 11A). This reversal was quantitatively similar to the inhibition of NGF-induced neuroprotection in PC12 cells, as previously reported (Yao, R. and Cooper, G., Science, 267:2003-2006 (1995)).

Wortmannin also reversed protection against apoptosis induced by the co-administration of TS-F and CNTF, although to a lesser extent than that of TS-F alone (FIG. 11A). For example, when PC12 cells were co-treated with Wortmannin (200 mM) and TS-F or TS-F+CNTF, neuronal viability was 57±2% and 80±3% of that observed with TS-F or TS-F+CNTF without Wortmannin, respectively (FIG. 11A). Because CNTF signaling does not appear to require PI-3 kinase activation (Inoue, M. et al., Mol. Neurobiol., 12:195-209 (1996)), the reduced efficiency of Wortmannin to inhibit neuroprotection produced by the TS-F+CNTF combination, compared to TS-F alone, is consistent with the view that TS signaling in PC12 cells requires PI-3 kinase activation. In addition, in agreement with published results (Zhong, W. et al., Brain Res., 661:56-62 (1994)), we found that CNTF by itself produced little, if any, protection of PC12 cells from undergoing apoptosis in serum-free medium (FIG. 11A).

The inhibition of TS-F-induced neuroprotection by Wortmannin was confirmed by experiments with LY294002, another PI-3 kinase inhibitor (Vlahos, C. et al., J. Biol. Chem., 269:5241-5248 (1994)). LY294002 induced apoptosis in PC12 cells maintained in TS-F or NGF in a concentration range (FIG. 11B) similar to the one effective in causing death of IGF-1-stimulated cerebellar granule neurons (D'Mello, S. et al., J. Neurosci., 17:1548-1560 (1997)). As with Wortmannin, LY294002 was less effective in reversing the protection of TS-F+CNTF than of TS-F only (FIG. 11B).

### TS Activates Protein Kinase Akt in Neuronal Cells

Treatment of TS-stimulated PC12 cells with the PI-3 kinase inhibitors Wortmannin and LY294002 induced apoptosis (FIGS. 11A and 11B), suggesting that PI-3 kinase may play an important role in TS-induced cell survival. Lipid products of PI-3 kinase activity directly activate the serine/threonine kinase Akt, which then becomes phosphorylated at threonine-308 and serine-473 by the protein kinase PDK1 and an unknown kinase, respectively (Franke, T. et al., Cell, 81:727-736 (1995), for reviews see Franke, T. et al., Cell, 88:435-437 (1997); Downward, J., Curr. Opin. Cell Biol., 10:262-267 (1998)). Activated Akt phosphorylates the Bcl-2 family member BAD and the Forkhead transcription factor, leading to cell survival (Datta, S. et al., Cell, 91:231-241 (1997); Brunet, A. et al., Cell, 96:857-868 (1999)).

Thus, if TS activates PI-3 kinase signaling pathway to promote survival, then the TS action on PC12 cells should result in the stimulation of Akt kinase. To determine whether Akt is activated in response to TS, we used an antibody specific for the Akt serine-473 epitope to detect activated Akt in TS-stimulated PC12 cells. The immunoblot displayed in FIG. 11C shows that Akt becomes activated after brief (2-5 minutes) exposure of PC12 cells to TS. The extent of TS-dependent Akt phosphorylation was similar to the phosphorylation produced by 20% fetal calf serum. The catalytic domain of TS (fragment of TS-F) was effective in activating Akt, whereas the C-terminal tandem repeat LTR fragment was not (FIG. 11D), consistent with PC12 cell survival being induced by TS-F, but not LTR. Furthermore, the PI-3 kinase inhibitor LY294002 completely blocked TS-F-induced Akt phosphorylation (FIG. 11D), consistent with a role for a TS-dependent PI-3 kinase activation of Akt, and analogous to the PI-3 kinase/Akt kinase activation by NGF, PDGF, IL-3 and other growth factors (Franke, T. et al., Cell, 88:435-437 (1997); Downward, J., Curr. Opin. Cell Biol., 10:262-267 (1998)).

### Discussion

The studies presented herein, demonstrate that TS can provide trophic support for neurons and glial cells (e.g., Schwann cells). Importantly, TS effectively supported the development and survival of neurons at low pM concentrations (FIGS. 3A, 4 and 5). In fact, TS was more potent on a molar basis then mammalian neurotrophic factors, such as NGF (FIG. 5). Such effective concentrations of TS can easily be achieved in vivo by administration of the protein. TS-modeled recombinant polypeptides and synthetic peptides which do not have neuraminidase/sialyltransferase catalytic activities were also found to be neurotrophic, clearly demonstrating that the neurotrophic activity of TS is not due to the catalytic activity of the protein.

The neurotrophic activity of TS, a trypanosome protein, can synergize with mammalian neurotrophic factors. Trophic support can be provided by co-administering TS and a mammalian neurotrophic factor (e.g., CNTF, LIF) at concentrations below the effective concentration of each factor when administered individually. Thus, co-administration of synergistic amounts of TS and a mammalian neurotrophic factor can provide effective trophic support with significantly reduced or no undesirable side effects.

TS can provide neurotrophic support for neurons directly and/or indirectly. The capacity of TS to protect PC12 cells from apoptosis was affected by the activity of phosphatidylinositol-3 kinase (PI-3 kinase). Similarly, the neuroprotective activity of NGF in PC12 cells (Yao, R., and Cooper, G., Science, 267: 2003-2006 (1995)) and of insulin-like growth factor (IGF) in cerebellar granule neurons (D'Mello, S. et al., J. Neurosci., 17:1548-1560 (1997)) requires phosphatidylinositol-3 kinase (PI-3 kinase). Thus, TS may bind to a neurotrophic surface receptor which activates a PI-3 kinase-dependent signaling pathway, as do NGF and IGF. In addition, TS potently and specifically induced IL-6 secretion in human intestinal microvascular endothelial cells and peripheral blood mononuclear cells and in mouse splenocytes. It is well established that IL-6 promotes survival of various types of neurons, including primary cultures of sympathetic neurons and PC12 cells (März, P. et al., Proc. Natl. Acad. Sci. USA, 95:3251-3256 (1998)). In addition, IL-6 can synergize with NGF and other neurotrophic factors to promote neuron survival and differentiation (Wu, Y., and Bradshaw, R., J. Biol. Chem., 271:13033-13039 (1996)). IL-6, like CNTF and other IL-6 family members, promotes differentiation and survival of neurons, not by activating the PI-3 kinase pathway, but by triggering the Janus kinase (JAK)/signal transducer and activator of transcription (STAT) signaling pathway (Nakashima, K. and Taga, T., Semin. Hematol., 35:210-221 (1998)). The possible convergence of various signaling pathways, whether activation of the PI-3 kinase, or synergism with CNTF, or indirectly through IL-6, is consistent with the exquisite sensitivity of PC12 cells to the neurotrophic action of TS.

The studies presented herein demonstrate that TS, active fragments and active peptides thereof can be used to provide trophic support for neurons in a mammal. The studies also provide an explanation for the existence of many TS family members which lack both neuraminidase and trans-sialidase activities (Uemura, H. et al., EMBO J., 11:3837-3844 (1992); Parodi, A. et al., EMBO J., 11:1705-1710 (1992)) some of which are found on multiple T. cruzi chromosomes (Henriksson, J. et al., Mol. Biochem. Parasitol., 42:213-224 (1990)). These proteins may function as neurotrophic factors which prevent destruction of the host's neurons and subsequent death of the host. This strategy would provide T. cruzi with additional opportunities to infect reduviid bugs feeding on the infected host, thereby completing the parasitic life cycle.

### EXAMPLE 2

### T. cruzi Infection, TS and Peptides Derived From TS Promote Survival of Human Schwann Cells

The following materials and methods were used:

### Cell Culture

Immortalized human Schwann cells (Rambukkana, A. et al., Science, 282:2076-2079 (1998)) were maintained in DMEM supplemented with 10% FCS, 0.5 mM pyruvate Na (Gibco) and 0.1 mM nonessential amino acids. Vero cell monolayers were grown at 37° C. in DMEM with 2.5% FCS, 100 U/ml penicillin and 100 μg/ml streptomycin in humidified chambers, as previously described (Pereira, M. et al. Infect. Immun., 64:3884-3892 (1996)).

### Parasites

T. cruzi trypomastigotes, Silvio strain, were maintained in Vero cell cultures, as described earlier (Pereira, M. et al., Infect. Immun., 64:3884-3892 (1996)). Trypomastigotes were collected 5 days after the start of infection and immediately used to infect Schwann cells. T. cruzi epimastigotes were grown in acellular LIT medium at 26° C. for 5-10 days (Saavedra, E. et al., J. Exp. Med., 190:1825-1836 (1999)).

For infection assays, Schwann cell monolayers were infected with T. cruzi at 2×10⁵ parasites/ml. After 2 hours, most swimming parasites were removed by washing and the cell monolayers were switched to serum-free medium for 72 hours. Intracellular parasites were identified after staining with Giemsa or by indirect immunofluorescence using chagasic IgG as primary antibody and Alexa 594-labeled second antibody, as described earlier (Ming, M. et al., Cell, 82:287-296 (1995)). Isolation of TS⁺ and TS⁻ trypomastigotes was based on the use of magnetic beads containing immobilized monoclonal antibody (mAb TCN-2) specific for the C-terminus tandem repeat of TS, as described earlier (Meciano Filho, J. et al., Gerontology, 41:18-21 (1995)). TS⁺ parasites were eluted from the beads with synthetic peptide hapten while the TS⁻ parasites were obtained by negative selection. The isolated sub-populations were checked for their specific TS activities and immediately used in infection and Akt assays.

Leishmania major promastigotes, strain Friedlin VI, (MHOM/IL/80/Friedlin) transfected with pXG1a and pXG1a-TS are clones L1D4-vector and L1D4-TS, respectively, as described earlier (Carrillo, M. B. et al., Infect. Immun., 68:2728-2734 (2000)); they were maintained at 26° C. in M199 medium (Gibco) supplemented with 10% FCS. For Akt activation experiments, trypomastigotes and epimastigotes of T. cruzi, and promastigotes of L. major, were washed 3× with serum-free DMEM and applied to monolayers of Schwann cells for predetermined periods of time.

### Purification of TS and TSA-1

TS was isolated from supernatants of Vero cells infected with T. cruzi by immuno-affinity chromatography as described in Example 1. The purified TS yielded a doublet of MW 200 kDa as determined by Coomassie-stained polyacrylamide gels (Scudder, P. et al., J. Biol. Chem. 268:9886-9891 (1993)). The recombinant catalytic domain of TS, CD (also referred to as TS-F or ΔTS), expressed in E. coli was purified by metal chelate and anion exchange chromatography as described in Example 1. CD derived from clone 19Y was used in the Schwann cell studies. LTR and TS-F-47 fragments of TS were isolated from engineered insect cells and bacteria, respectively, as described in Example 1.

TS enzymatic activity was measured by the sialylation of the acceptor ¹⁴C-labeled N-acetyllactosamine on anion exchange resins (Scudder, P. et al., J. Biol. Chem. 268:9886-9891 (1993)). Recombinant TSA-1, was expressed on insect sf9 cells and purified from serum-free medium by ion exchange chromatography (monoQ) and preparative SDS-PAGE (Wrightsman, R. A. et al., J. Immunol., 153:3148-3154 (1994).

### Identification of Apoptotic Nuclei

Schwann cells were plated to 70% confluency in 16-well chamber slides (LabTek) in DMEM with 10% FCS. After overnight incubation at 37° C., medium was changed to serum-free DMEM without or with TS or other test compounds. Cells were fixed for 24-72 hours later with 1% paraformaldehyde overnight at 4° C. and stained with 4′, 6-diamidino-2-phenylindole (DAPI), 2 μg/ml in PBS, pH 7.2, for 2 min. For quantification, normal, condensed and fragmented nuclei in 10 randomly chosen fields and accounting more than 400 cells were counted at ×40 magnification in triplicate samples, in a minimum of 3 assays.

### Immunodetection of Activated Akt

Schwann cells, grown in 10% FCS to 70% confluency in 6-well plates, were switched to DMEM containing 0.1% FCS for 48 hours to reduce basal Akt phosphorylation. Then the cells were placed in serum-free DMEM medium for 2 hours and challenged with TS, CD or other test factors for predetermined periods of time. When the PI-3 kinase inhibitor LY294002 (Sigma) was used, the inhibitor was added to the serum-starved cells 30 minutes before challenge with TS or CD. Cell samples were lysed with lysis buffer (20 mM Tris, pH 7.5, 150 mM NaCl, 1 mM EDTA, 1% Triton X-100 (t-Octylphenoxypolyethoxyethanol), 2.5 mM sodium pyrophosphate, 1 mM glycerophosphate, 1 mM Na₃VO₄, 1 μg/ml leupeptin and 1 mM Phenymethylsulfonyl fluoride (PMSF)). Samples (40 μg) were run on 10% SDS-Page, blotted to nitrocellulose paper and analyzed by Western (immuno) blot with monoclonal antibody specific for P-Ser473 of Akt (New England Biolabs). Reaction was visualized by chemiluminescence using the ECL kit (New England Nuclear).

### Kinase Assay of Akt In Vitro

Enzymatic activity of Akt kinase was assessed using the synthetic substrate GSK-3α-fusion protein (Cell Signaling Technology, Beverly, Mass.), according to the protocol provided by Cell Signaling Technology. In short, lysates of Schwann cells subjected to various treatments were immunoprecipitated with Akt antibody coupled to agarose beads, and the immunoprecipitates incubated with GSK-3α-fusion protein as the substrate for Akt phosphorylation, in the presence of 200 μM ATP. Phosphorylation of GSK-3 fusion protein was detected by Western blot using phospho-GSK-3αβ (Ser21/9)-specific antibody (Cell Signaling Technology). The reaction product was identified by chemiluminescence using the ECL kit (New England Nuclear).

### Generation of Stabel Tetracycline-regulated AktKI/GFP and Transient PTEN Transfectants

A plasmid encoding the hemagglutinin (HA)-tagged Akt rendered kinase inactive (AktKI) by a mutation in its ATP binding site (K179M), was provided by Drs. Alfonso Bellacosa (Fox Chase Cancer Center, Philadelphia) and Lewis Cantley (Beth Israel Hospital, Harvard Medical School) (Skorski, T. et al., EMBO J., 16:6151-6161 (1997)). The plasmid pTR5-AktKI/GFP was constructed by subcloning AktKI into the PmeI site of the tetracycline-regulated dicistronic expression plasmid pTR5-DC/GFP (Mosser, D. D. et al., Biotechniques, 22:158-161 (1997)). To generate cells expressing the tetracycline-regulated transactivator protein tTA, which allows control of gene expression by tetracycline, Schwann cells were transfected with PtTA-Hygro using SuperFect™ transfection reagent (Qiagen, Valencia, Calif.) and selected for hygromycin B resistance, as described (Hall, B. S. et al., Mol. Biol. Cell, 11:153-160 (2000)). Positive cells were isolated by fluorescent-activated cell sorting (FACS) after transfection with pTR-GFP, an expression plasmid encoding green fluorescence (GFP) under the control of the tet operator. These cells were co-transfected with pTR5-AktKI/GFP and pcDNA3 and selected for neomycin resistance in the presence of 1 μg/ml Geneticin (Life Technologies). In addition, 50 ng/ml tetracycline was included in the medium to prevent expression of AktKI during selection. Cells expressing AktKI and GFP were selected by removal of tetracycline followed by FACS. Cells were maintained in medium containing 10% FCS and 50 ng/ml tetracycline and transferred to tetracycline-free medium 24 hours (or otherwise indicated) before assay.

The plasmids pcDNA3-PTEN and pcDNA3-G129R encoded wild type PTEN and phosphatase-inactive mutant of PTEN, respectively (Furnari, F. B. et al., Proc. Natl. Acad. Sci. USA, 94:12479-12484 (1997); Myers, M. P. et al., Proc. Natl. Acad. Sci. USA, 94:9052-9057 (1997)). Schwann cells were seeded in 10% FCS to 50-70% confluency in 10 cm Petri dishes 24 hours prior transfection, performed in serum-free DMEM. Cells were maintained in 10% FCS for 24 hours and then switched to medium containing 700 μg/ml G418 (Gibco BRL) and 2% FCS for 3 days and 0.1% FCS for another 4 days with CD added to some cell monolayers. After this cells were assessed for viability by Trypan blue exclusion and by DAPI staining. At the same time cell lysates were analyzed for Akt phosphoryltion by Western (immuno) blots with monoclonal antibody specific for P-Ser473 of Akt (New England Biolabs). Reaction was visualized by chemiluminescence using the ECL kit (New England Nuclear).

### Results

T. cruzi Invasion Blocks Apoptosis of Schwann Cells:

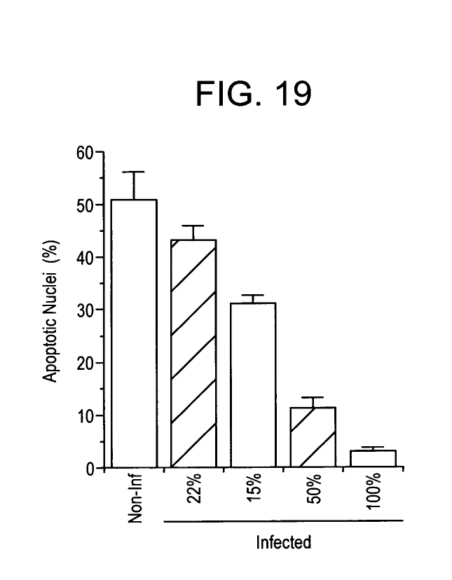

Little cell death (<1%; detected by nuclear staining with DAPI) was detected in cultures of immortalized Schwann cells that were maintained in medium containing 2% FCS for 72 hours. However, immortalized Schwann cells underwent apoptosis in a time-dependent manner when cultured in medium devoid of serum. About 50% of cells in such cultures contained fragmented (apoptotic) nuclei by 72 hours (FIG. 19, bar labeled ‘Non-Inf’). Primary cultures of rat Schwann cells are reported to have similar susceptibility to cell death induced by serum-withdrawal (Delaney, C. L. et al., Neurobiol., 41:540-548 (1999); Weiner, J. A. and Chun, J., Proc. Natl. Acad. Sci., 96:5233-5238 (1999); Campana, W. M. et al., J. Neurosci. Res., 57:332-341 (1999)).

To further study the survival-promoting effect of T. cruzi, Schwann cell monolayers were exposed to trypomastigotes (the infective stage of T. cruzi) for about two hours, after which most of the swimming parasites were removed by washing. The resulting infected monolayers and non-infected monolayers were cultured for 72 hours in media that did not contain serum. Under the assay conditions used, about 23±3% of Schwann cells harbored intracellular T. cruzi (amastigotes). This rate of infection is similar to the rate of infection reported for myocytes (Hall, B. S. et al., Mol. Biol. Cell, 11:153-160 (2000)). Inspection of the resulting monolayers using phase contrast microscopy revealed that Schwann cells in the infected monolayers looked more viable than cells in the non-infected monolayers, as assessed by cell detachment from the substratum, membrane blebbing, and cellular extensions. The infected Schwann cell monolayers were further analyzed by staining with DAPI and with chagasic IgG which were employed to detect apoptotic nuclie and intracellular infection (Ming, M. et al., Cell, 82:287-296 (1995)), respectively. This analysis revealed an inverse correlation between the proportion of Schwann cells with fragmented nuclei and Schwann cells with intracellular T. cruzi (FIG. 19). Virtually all Schwann cells that contained intracellular parasites did not exhibit apoptotic nuclei despite being starved for 3 days in serum-free medium. In contrast, a high proportion of Schwann cells that were not infected with T. cruzi had apoptotic nuclei (FIG. 19). These data demonstrate that T. cruzi invasion protects Schwann cells against apoptosis induced by serum starvation.

Invasive, But Not Noninvasive, T. cruzi, Enhance Activation of Akt Kinase in Human Schwann Cells:

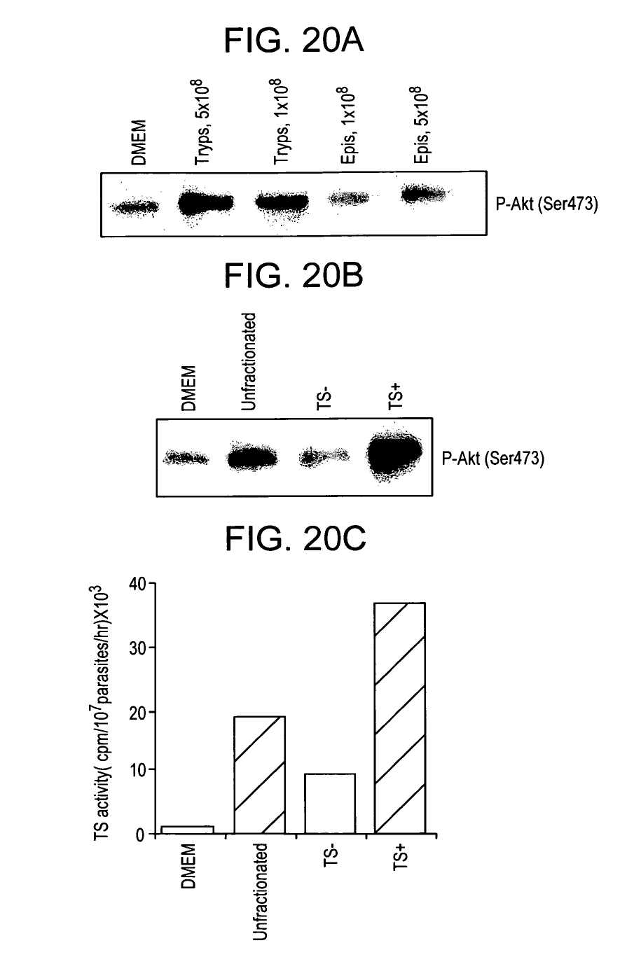

To determine whether T. cruzi exploits the Akt kinase to protect Schwann cells against apoptosis, monolayers of Schwann cells maintained in serum-free medium were exposed to trypomastigotes or epimastigotes, which are invasive and noninvasive stages of T. cruzi, respectively. After 10 minutes of exposutre, parasites that were not attached to the monolayers were removed by washing, the cells were lysed in lysis buffer, and the resulting lysates were analyzed for the presence of phosphorylated Akt kinase (phosphorylated on Ser-473) by immunoblotting (Datta, S. R. et al., Genes Dev., 13:2905-2927 (1999)). The results of the immunoblotting studies clearly showed that invasive trypomastigotes (Tryps) potently activated Akt kinase in a dose-dependent manner, while noninvasive epimastigotes (Epis) did not detectably activate Akt (FIG. 20A) under similar conditions.

Trypomastigotes consist of two morphologically similar sub-populations, TS⁺ and TS⁻, which have remarkably contrasting abilities to invade cells, with the former being strongly, and the latter weakly invasive for mammalian cells (Pereira, M. E. A. et al., Infect. Immun., 64:3884-3892 (1996), Saavedra, E. et al., J. Exp. Med., 190:1825-1836 (1999)). The TS⁺ and TS⁻ sub-populations differ in expression of surface-located T. cruzi-specific ligand, enzyme trans-sialidase, TS (Schenkman, S. et al., Annu. Rev. Microbiol., 48:499-523 (1994)). To determine if trypomastigote-induced activation of Schwann cell Akt kinase is restricted to the subpopulation of parasites with enhanced invasiveness and TS expression (i.e., TS⁺ parasites), the generation of phosphorylated Akt in Schwann cells infected for 20 minutes with unfractionated trypomastigotes or with trypomastigotes of the TS⁺ or TS⁻ phenotypes was monitored. The strongly invasive TS⁺ trypomastigotes were more potent in activating Akt kinase than unfractionated trypomastigotes (FIGS. 20B and 20C), of which only ˜20-30% of organisms are of the TS⁺ phenotype (Pereira, M. E. A. et al., Infect. Immun., 64:3884-3892 (1996), Saavedra, E. et al., J. Exp. Med., 190:1825-1836 (1999)). On the other hand, infection of Schwann cells with noninvasive TS⁻ trypomastigotes did not activate Akt kinase under the same conditions (FIGS. 20A and 20B). These results demonstrate that T. cruzi invasion of Schwann cells can lead to activation of Akt kinase.

### TS, Through its Catalytic Domain, is a Potent, Specific Survival Factor for Schwann Cells and an Activator of PI3K/Akt Kinase Signaling.

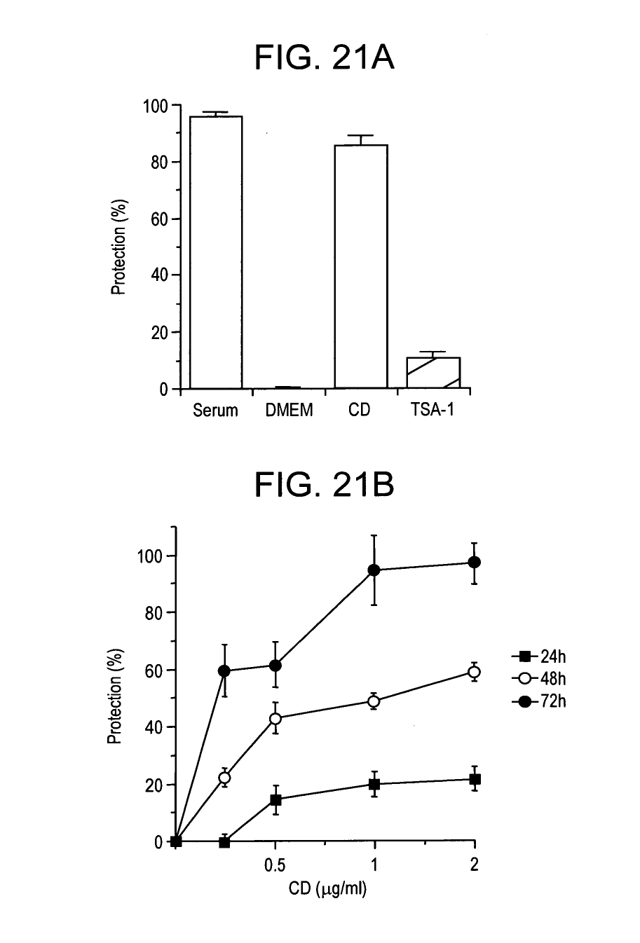

The results of the infection studies with TS⁺ and TS⁻ trypomastigotes and of the studies described in Example 1, indicated that the T. cruzi sialidase is a ligand that promotes survival and activation of PI3K/Akt in Schwann cells. Therefore, TS purified from T. cruzi TS was tested for the capacity to inhibit apoptosis of Schwann cells induced by serum starvation. The addition of TS to Schwann cells subjected to serum starvation promoted survival of the cells and activated PI3K/Akt signaling at a dose as low as 100 ng TS per milliliter of medium (0.5 nM TS). Furthermore, a bacterially-expressed N-terminal fragment of TS consisting of the catalytic domain (CD; residues 33-666 of SEQ ID NO:2) protected Schwann cells against apoptosis (FIG. 21A) in a time- and dose-dependent manner (FIG. 21B), while the counterpart C-terminal long tandem repeat (LTR) did not promote Schwann cell survival nor activate PI3K/Akt signaling. Therefore, the catalytic domain of TS is the domain of the protein which has survival promoting activity for Schwann cells. Similarly, the catalytic domain (CD) was the moiety of TS that promoted the survival of PC12 cells (Example 1).

Strikingly, the presence of CD at 500 ng/ml (6 nM) in serum-free DMEM was nearly as effective in protecting Schwann cells from apoptosis as 2% serum (FIG. 21A). This result underscores the potency of TS as a survival factor for Schwann cells.

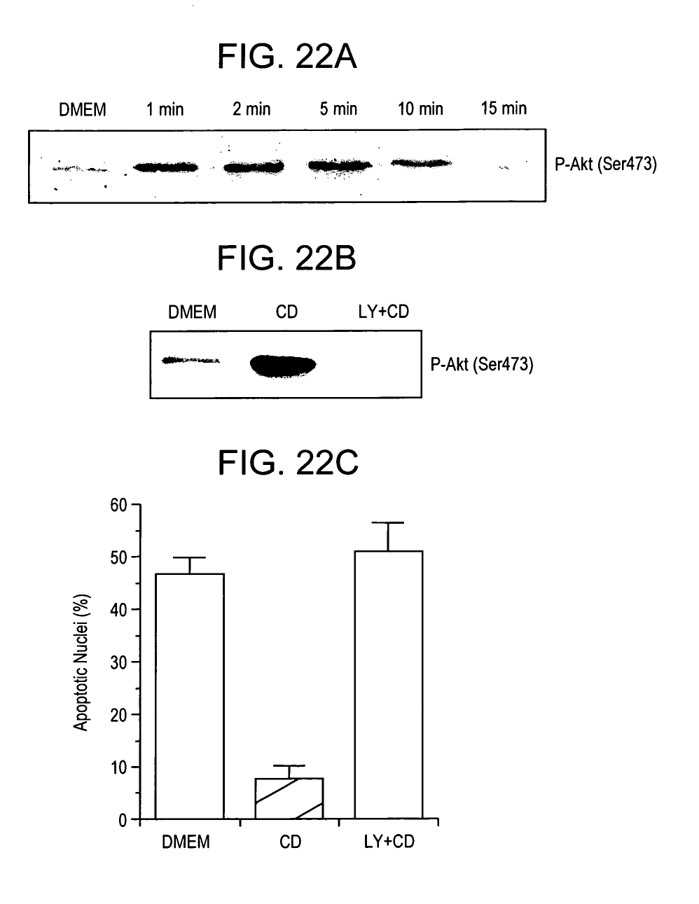

In addition, CD rapidly (within 1-5 min) and transiently (phospho-Akt not detected after 15 min) activated Schwann cell Akt kinase (FIG. 22A). Ly294002, a selective inhibitor of PI3K (Vlahos, C. J. et al., J. Biol. Chem., 269: 5241-5248 (1994)), blocked CD-induced Akt activation (FIG. 22B) and inhibited TS-induced survival of serum starved Schwann cells (FIG. 22C), indicating that TS induces phosphorylation of Akt kinase via activation of upstream PI3K.

A T. cruzi surface antigen, TSA-1, that belongs to the TS superfamily (Chuenkova, M. et al., Biochem. Biophys. Res. Commun., 262:549-556 (1999)) (FIG. 3B, insert), did not protect Schwann cells against apoptosis (FIG. 21A), nor did it activate Akt kinase under the conditions TS and its catalytic domain did. These results emphasize the specificity of the anti-apoptotic action of TS.

Heterologous Expression of TS in Leishmania major Converts the Otherwise Inactive Parasites into Activators of Akt Kinase in Schwann Cells.

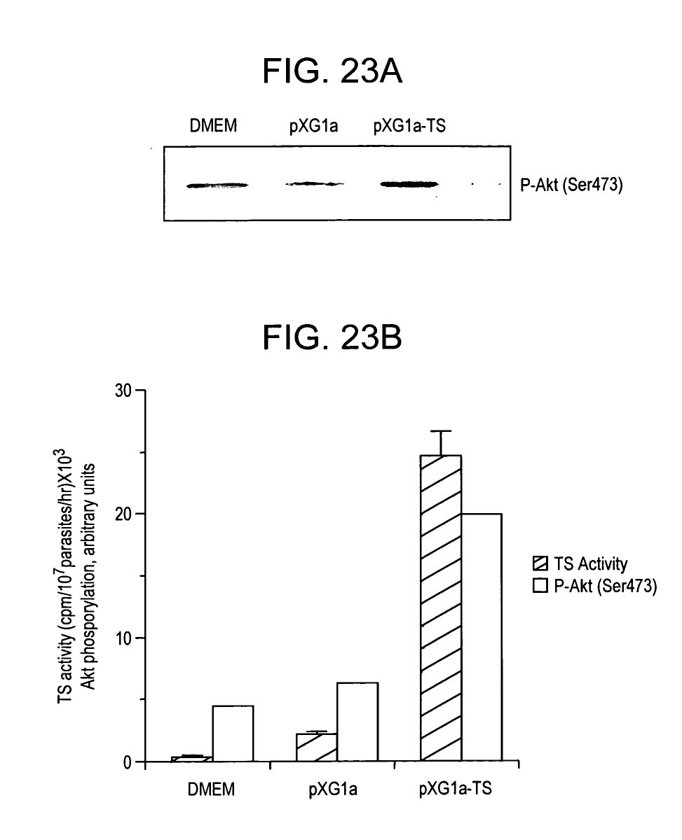

L. major is a protozoan parasite that selectively invades macrophages and cause cutaneous leishmaniasis worldwide. L. major, unlike T. cruzi, does not express TS (Belen Carrillo, M. et al., Infect. Immun., 68:2728-2734 (2000)). However, L. major transfected with the constitutive expression vector pXGla containing the full-length coding region of TS (pXG1a-TS), produces a recombinant surface-located protein with enzymatic (FIG. 23B), immunological, and virulence-enhancing activities similar to the endogenous T. cruzi enzyme (Belen Carrillo, M. et al., Infect. Immun., 68:2728-2734 (2000)). Therefore, human Schwann cells were co-cultured with L. tropica that were transfected with pXG1a-TS and expressed T. cruzi TS or with L. tropica that were transfected with the empty vector (pXG1a). Co-culturing Schwann cells with L. tropica that did not express TS (L. tropica transfected with vector pXG1a) did not result in detectable activation of Schwann cell Akt kinase (FIG. 23A). However, Schwann cell Akt kinase was activated in co-cultures that contained L. tropica that expressed T. cruzi TS (FIG. 23A). These results further establish that TS is a trigger of PI3K/Akt signaling in human Schwann cells.

### TS Does Not Promote Survival of Schwann Cells Whose PI3K/Akt Signaling is Inactivated

To further investigate the link between TS-induced survival of Schwann cells and the PI3K/Akt signaling pathway, Schwann cells capable of inducible expression of a kinase-inactive, dominant negative mutant, of Akt (HA-Akt(K179M); also referred to as AktKI) were used (Skorski, T. et al., EMBO J., 16:6151-6161 (1997)). To produce such cells, Schwann cells were transfected with ptTA-hygro a plasmid encoding a tetracycline-regulated transcriptional activator (tTA) (Mosser, D. D. et al., Biotechniques, 22:158-161 (1997); Hall, B. S. et al., Mol. Biol. Cell, 11:153-160 (2000)). Following selection, cells transfected with ptTA-hygro were transfected with pTR5-GFP and pTR5-AktKI/GFP (expression plasmids that encoded green fluorescent protein (GFP) and AktKI+GFP, respectively in a dicystronic cassette under the control of a promoter containing the tet operator sequence). Stable transfectants exhibited tetracycline-repressible AktKI and GFP expression as determined by immunoblot and fluorescence, respectively. AktKI-expressing Schwann cells (grown in medium without tetracycline) exhibited apoptotic morphology even in serum-containing medium, while counterpart Schwann cells transfected with GFP alone did not.

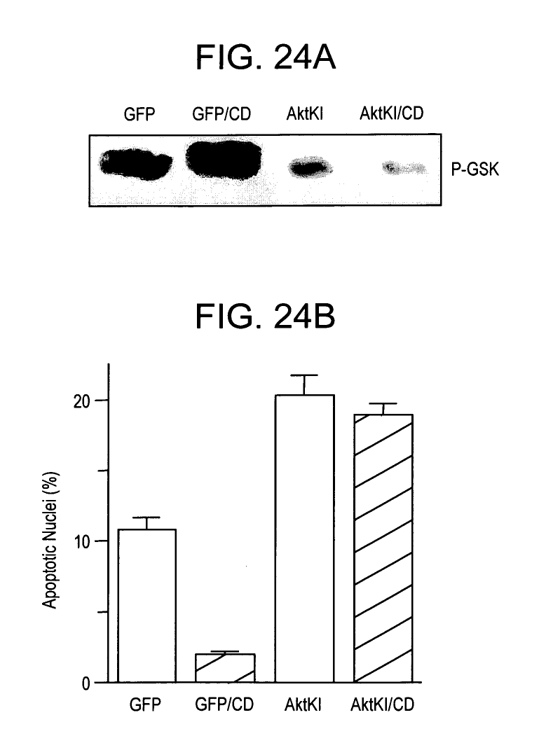

Akt immunoprecipitated from AktKI transfectants grown in medium without serum (to induce apoptosis) and tetracycline (to induce expression of AktKI and GFP) exhibited dramatically low background kinase activity towards GSK-3a substrate, relative to kinase activity of Akt immunoprecipitated from Schwann cells transfected with GFP alone (FIG. 24A). Furthermore, serum-starved AktKI-transfected Schwann cells exhibited higher levels of apoptosis than counterpart Schwann cells transfected with GFP alone (FIG. 24B). These findings demonstrate the dominant negative action of AktKI, and further demonstrate that Schwann cell survival depends on PI3K/Akt signaling (Delaney, C. L. et al., Neurobiol., 41:540-548 (1999); Weiner, J. A. and Chun, J., Proc. Natl. Acad. Sci., 96:5233-5238 (1999); Campana, W. M. et al., J. Neurosci. Res., 57:332-341 (1999)).

Whether CD could inhibit serum starvation-induced apoptosis of Schwann cells expressing the dominant negative AktKI was also investigated. The addition of CD to serum-starved AktKI-transfected Schwann cells did not increase survival of the cells. However, the addition of CD to serum-starved control Schwann cells transfected with GFP vector (FIG. 24B) did increase survival of the cells. The inability of CD to promote survival of Schwann cells that over expressed dominant negative AktKI was accompanied by a similar inability of CD to enhance endogenous Akt enzymatic activity, contrary to the stimulation of Akt activity in control GFP-transfected Schwann cells (FIG. 24A).

To further establish the relationship between TS-induced survival and activation of PI3K/Akt in Schwann cells, Schwann cells that overexpressed the PI3K antagonist PTEN (Cantley, L. and Neel, B. G., Proc. Natl. Acad. Sci. USA, 96:4240-4245 (1999) were used. PTEN dephosphorylates the 3 position of phosphoinosites generated by PI3K and thus downregulates activation of Akt, and consequently, Akt-dependent cell survival (Furnari, F. B. et al., Proc. Natl. Acad. Sci. USA, 94:12479-12484 (1997); Wu, X. et al., Proc. Natl. Acad. Sci. USA, 95:15587-15591 (1998); Cantley, L. and Neel, B. G., Proc. Natl. Acad. Sci. USA, 96:4240-4245 (1999).

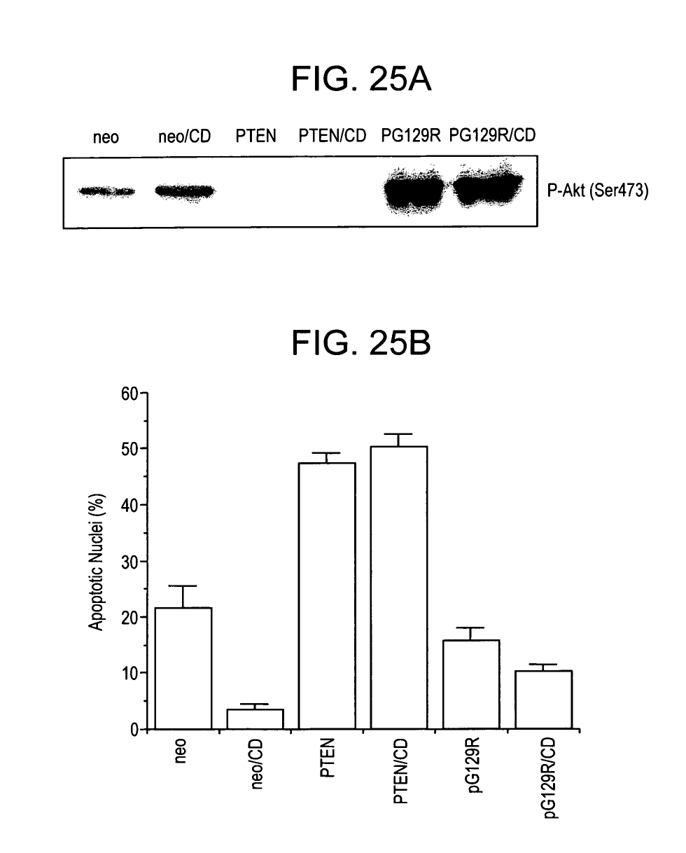

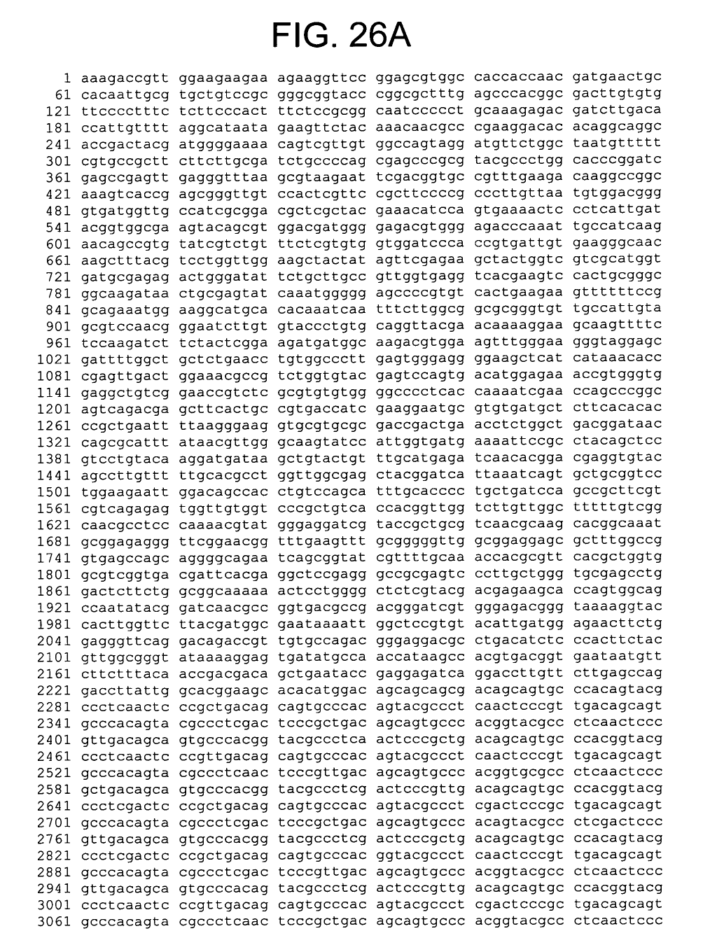

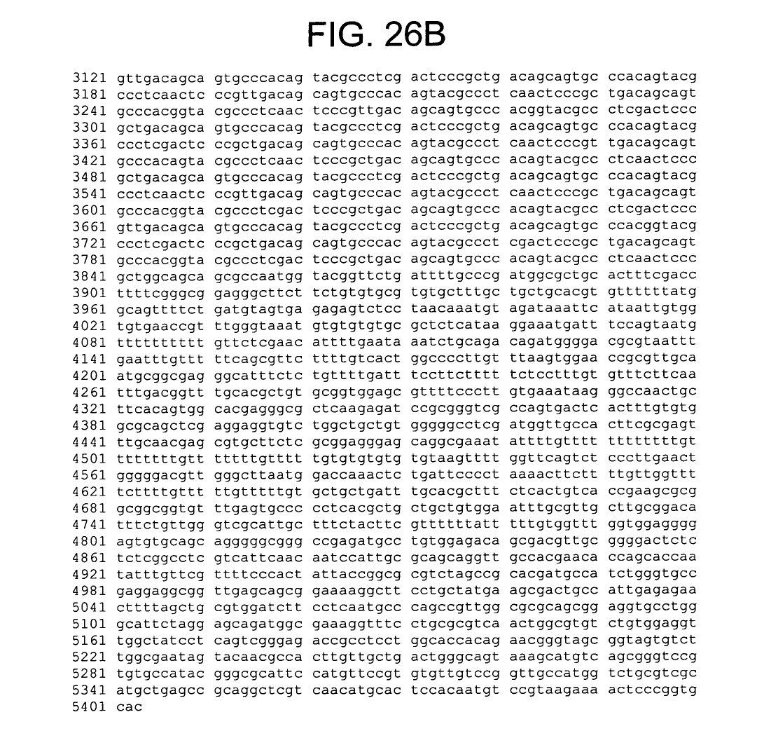

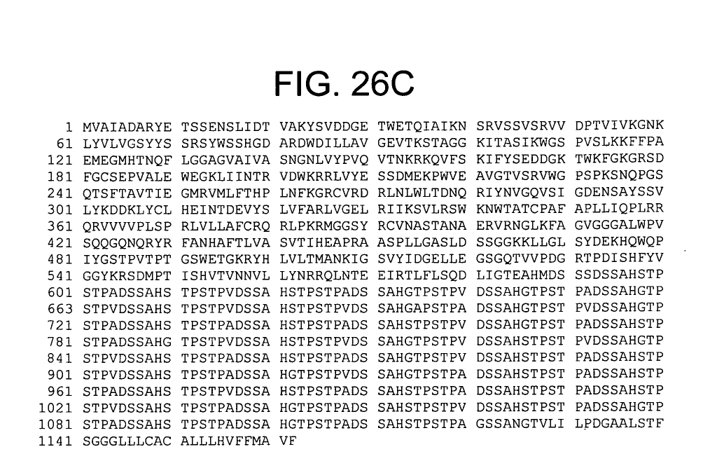

The addition of CD to Schwann cells transfected with PTEN did not result in the generation of phospho-Akt (FIG. 25A), and did not rescue cells from apoptotic death (FIG. 25B). In contrast, CD boosted levels of phospho-Akt and decreased the number of fragmented nuclei by about ten-fold in serum-starved Schwann cells transfected with empty vector (neo) (FIGS. 25A and 25B). Moreover, Schwann cells transfected with a dominant negative mutant of PTEN (pGG129R) (Furnari, F. B. et al., Proc. Natl. Acad. Sci. USA 94:12479-12484 (1997)) had an extremely high background of phosphorylated Akt and a low degree of apoptotic nuclei relative to control cells transfected with empty pcDNA3 vector (neo) (FIGS. 25A and 25B). However, CD did not detectably enhance Akt phosphorylation and survival in these transfectants, as it did with Schwann cells transfected with empty vector (FIGS. 25A and 25B). These results establish that the PI3K/Akt signalling pathway can be involved in T. cruzi TS-promoted survival of Schwann cells.

In further studies, peptides C44 (SEQ ID NO:12), C14 (SEQ ID NO:14), CFN1 (SEQ ID NO:13), C19Y21 (SEQ ID NO:15), CYN2 (SEQ ID NO:16), CYNF (SEQ ID NO:17), B2 (SEQ ID NO:18) or TR (SEQ ID NO:19) were tested for the capacity to inhibit apoptosis of PC12 cells or Schwann cells. Peptides C44 (SEQ ID NO:12), C14 (SEQ ID NO:14), CFN1 (SEQ ID NO:13), C19Y21 (SEQ ID NO:15) were potent suppressors of apoptosis in the assays. Peptide CYN2 (SEQ ID NO:16) also suppressed apoptosis but was less active than peptides C44 (SEQ ID NO:12), C14 (SEQ ID NO:14), CFN1 (SEQ ID NO:13), C19Y21 (SEQ ID NO:15). The appoptosis suppressing effect of peptides C44 (SEQ ID NO:12), C14 (SEQ ID NO:14), CFN1 (SEQ ID NO:13), C19Y21 (SEQ ID NO:15), CYN2 (SEQ ID NO:16) and CYNF (SEQ ID NO:17) was inhibited by the PI-3 kinase inhibitor LY294002. Peptides CYNF (SEQ ID NO:17), B2 (SEQ ID NO:18) and TR (SEQ ID NO:19) did not inhibit apoptosis in the assays.

### Discussion

The results presented here indicate that T. cruzi invasion of the nervous system may keep destruction of neurons and glial cells in check (Chuenkova, M. V. and Pereira, M. A., Mol. Biol. Cell, 11:1487-1498 (2000)). Indeed, the results described herein establish that T. cruzi infection suppresses induced apoptosis of Schwann cells (FIG. 19). The T. cruzi-specified survival factor was identified to be their sialidase/sialyl transferase (TS) (FIGS. 20A-20C, 21A and 21B). TS is strategically localized to interact with mammalian cells, as it is present both on the parasite outer membrane (Prioli, R. P. et al., Trop. Med. Parasitol., 42:146-150 (1991)) and in the extracellular milieu as a water-soluble ligand (Cavallesco, R. and Pereira, M. E. A., J. Immunol., 140: 617-625 (1988), Pereira, M. E. A. et al., J. Exp. Med., 174:179-191 (1991)). On the surface membrane, TS facilitates binding of trypanosomes to surface receptors of host cells as a prelude to invasion (Ming, M. et al., Mol. Biochem. Parasitol., 59:243-252 (1993)), while in the extracellular environment it expedites reaction with cells that are not permissive to invasion, such as endothelial cells, neurons and lymphocytes (Saavedra, E. et al., J. Exp. Med., 190:1825-1836 (1999), Chuenkova, M. V. and Pereira, M. A., Mol. Biol. Cell, 11: 1487-1498 (2000)). Because TS can provide trophic support for neuronal cells (e.g., neurons and glial cells) and in keeping with the nomenclature adopted for certain molecules from virus and bacteria, named virokines and bacteriokines (Wilson, M. et al., Infect. Immun., 66:2401-2409 (1998)), respectively, the term ‘protokine’ (protozoan cytokine) is used to refer to a novel class of biologically active protozoan-derived molecules (Saavedra, E. et al., J. Exp. Med., 190:1825-1836 (1999)).

T. cruzi, particularly the invasive subset that expresses high levels of TS (TS+trypomastigotes), as well as TS isolated from T. cruzi and the TS catalytic domain expressed in bacteria, activated Schwann cell PI3K/Akt signaling (FIGS. 20A-20C and 22A-22C). Such signaling seems to be the mechanism underlying the anti-apoptotic action of T. cruzi because Schwann cells no longer respond to TS when their endogenous PI3K/Akt cascade was inactivated by transfection with relevant inhibitors (FIGS. 24A, 24B, 25A and 25B). In addition to Schwann cells, TS promoted survival and neurite outgrowth of the neuronal PC12 cells and of primary cultures of cerebellar granule neurons in a PI3K/Akt-dependent manner (Example 1). Thus, it is clear that the mechanism of Schwann cell protection induced by T. cruzi and the protokine TS involved PI3K/Akt signaling. Therefore, the protokine TS triggers signal transduction cascades similar to those of authentic mammalian cytokines, like IL-3 (Songyang, Z. et al., Proc. Natl. Acad. Sci. USA, 94:11345-11350 (1997). T. cruzi infection of macrophages triggers PI3K activation as well (Todorov, A. G. et al., J. Biol. Chem., 275:32182-32186 (2000)), though the T. cruzi-derived activating factor for this cell type has not been identified.

TS proved to be a strikingly potent trophic factor for Schwann cells and showed efficacy at a dose of about ≧0.5 nM, which is similar to the effective doses reported for other Schwann cell survival factors such as neuroregulin (Greespan, J. B. et al., J. Neurosci., 16:6107-6118 (1996)) and lysophosphatidic acid, which has been reported to be active at a dose of ≧10 nM for primary cultures of rat Schwann cells (Weiner, J. A. and Chun, J., Proc. Natl. Acad. Sci., 96:5233-5238 (1999)). Such potency was evident in the survival of PC12 cells as well, as TS effectively protected the neuronal cells at concentrations lower than those required by nerve growth factor (Example 1).

The neurotrophic action of TS is specific because other T. cruzi proteins such as the heparin-binding protein penetrin (Ortega-Barria, E. and Pereira, M. E. A., Cell, 67:411-421 (1991)), the protease cruzipan (Murta, A. C. et al., Mol. Biochem. Parasitol, 43:27-38 (1990)), and the TS superfamily member TSA-1 (Wrightsman, R. A. et al., J. Immunol., 153:3148-3154 (1994)) (FIG. 3), were all ineffective (Chuenkova, M. V. and Pereira, M. A., Mol. Biol. Cell, 11:1487-1498 (2000)).

The potent and specific survival promoting activity of T. cruzi TS for cells of the nervous system may be relevant to the pathogenesis of Chagas' disease. T. cruzi invasion of the nervous system is restricted largely to penetration and subsequent parasite development in glial cells (i.e., Schwann cells) in the peripheral nervous system (PNS) and astrocytes in the central nervous system (CNS) (Da, J. R et al., Brain Res. Bull., 53:153-162 (2000); Tafuri, W. L., Am. J. Trop. Med. Hyg., 19:405-417 (1970); McCabe, R. E. et al., Exp. Parasitol, 68:462-469 (1989); Tanowitz, H. B. et al., Am. J. Trop. Med. Hyg., 31:1090-1097 (1982); Wong, W. C. et al., Histol. Histopathol., 7: 371-378 (1992)). The majority of individuals infected with T. cruzi remain asymptomatic for years or decades and may show evidence of peripheral neuroregeneration, particularly in the GI tract and heart (Köberle, F., Parasitol, 6:63-71 (1968)). Animal models of Chagas' disease may also present signs of neurite growth (Losavio, A. et al., Am. J. Trop. Med. Hyg., 41:539-547 (1989)). However, ganglia in the GI tract and heart suffer extensive damage in the relatively few (<15%) patients who progress from the asymptomatic to the chronic stage of Chagas' disease (Andrade, Z. A., Ciba Found Symp., 99:214-33 (1983), Oliveira, J. S. M. et al., Am. Heart J., 109:304-308 (1985)). Such damage is most certainly a cause of cardiomegaly, megaesophagus and megacolon in the chronic patients.

Therefore neuron survival in the PNS is a critical event for the healthy status of chagasic patients, and as described herein, T. cruzi TS plays a role in neuroregeneration. Thus, T. cruzi TS can result in the prevention of pathology in asymptomatic individuals with Chagas' disease. Consequently, TS and neurotrophic fragment thereof can be used as therapeutics for treating Chagas disease and/or other neurodegenerative diseases.

PI3K signaling has been implicated in some bacterial infections of mammalian cells, specifically Listeria monocytogenes and E. coli invasion of epithelial cells and brain microvascular endothelial cells (Ireton, K. et al., Science, 274:780-782 (1999), Reddy, M. A. et al., J. Biol. Chem. 275: 36769-74 (2000)), respectively, as well as Cryptosporidiumparvum (Formey, J. R. et al., Infect. Immun., 67:844-852 (1999)). Thus, PI3K signaling may be a common mechanism that these and perhaps other microbes use to invade cells. The results of Examples 1 and 2, suggest that T. cruzi-induced PI3K/Akt activation helps establish parasitism in mammalian hosts by preventing or reducing damage to the nervous system.

### EXAMPLE 3

### TS and Peptides Derived from the Tandem-Repeat Domain of TS Induces the Secretion of IL-6

### Additional Materials and Methods

### Cell Culture

Primary cultures of human intestinal microvascular endothelial cells (HIMEC) isolated from normal jejunal mucosa/submucosal tissue were prepared as described (Strong et al., Gastroenterology 114:1244-1256 (1998)). The HIMEC were cultured in fibronectin-coated plasticware in MCDB medium (Sigma) supplemented with 20% FBS, 90 μg/ml heparin and 50 μg/ml endothelial cell growth factor (Sigma). T-24 cells (ATCC Accession No. HTB-4) were cultured in M199 medium supplemented with 10% FBS. PBMC were purified by Ficoll-Paque gradient as described (Current Protocols in Immunology, pp 7.1.1-7.1.2). Vero cells (ATCC Accession No. CCL-81) were grown in RPMI medium with 5% Nu serum and infected with the Silvio X-10/4 of T. cruzi as described previously (Chuenkova et al., J. Exp. Med. 181:1693-1703 (1995)).

### Cloning and Expression of Catalytic and Long Tandem Repeat Domains of TS

The catalytic domain of TS (TS-F, also referred to as CD) was produced as described in Example 1. The full-length C-terminal long tandem repeat (LTR fragment) of TS from T. cruzi clone 7F was isolated as follows: DNA encoding LTR, subcloned from pMelBac plasmid (Invitrogen) containing the TS gene, was digested with Pvu II/Sal I and ligated into EcoR V/Sal I sites of pET20b (Novagen). The LTR encoding DNA was then excised from pET20b using Nco I/Hind III and introduced into the Nco I/Hind III sites of pFASTBAC HTb (Gibco BRL). The Bac-to-Bac system (Gibco BRL) was used to generate recombinant baculovirus, which were used to infect Sf9 cells. Recombinant LTR protein was purified by Ni²⁺-NTA column affinity chromatography (Novagen). The LTR fragment contained the full-length tandem repeat domain of TS from clone 7F (44 repeats) with the 26 amino-terminal and 40 carboxyl-terminal amino acids which flank the repeat domain. Lipopolysaccharide was removed from the purified LTR protein by AffinityPak Detoxi-Gel chromatography.

### Preparation of TS-154 and TS-H32Constructs.

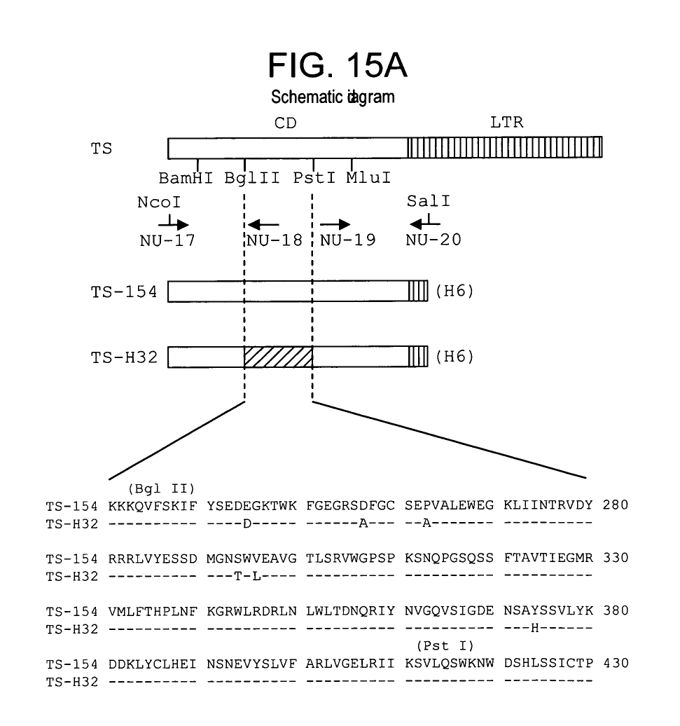

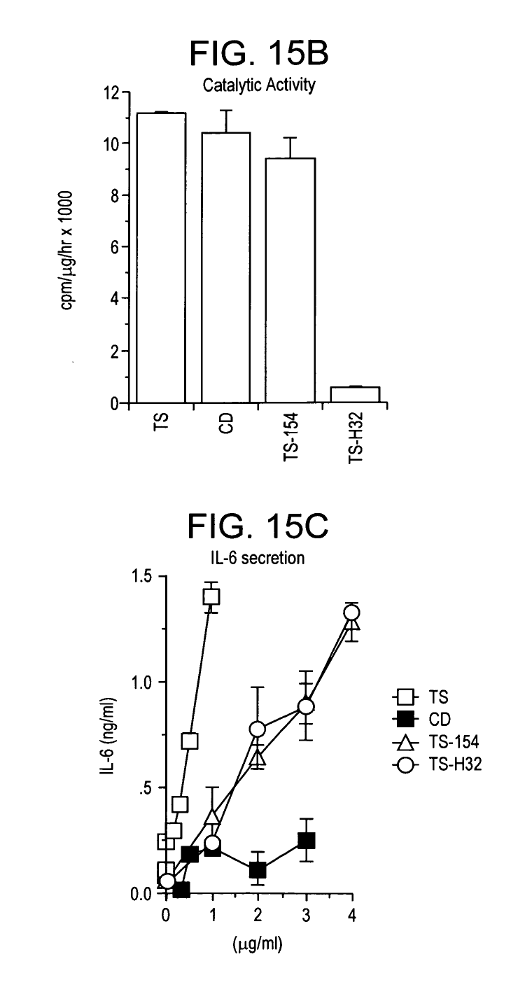

Construct TS-154 was derived from enzymatically active trans-sialidase clone 154 of the Y strain of T. cruzi (Uemura et al, EMBO J. 11:3837-3844). The N-terminus of clone 154 was amplified by PCR using synthetic DNA primers NU-17 (5′-GCCCATGGCACCCGATCGAGCCGAGTT-3′, SEQ ID NO:20) and NU-18 (5′-CGGAATTTTCATCACCAATG-3′, SEQ ID NO:21) which contained Nco I and Bgl II sites, respectively. NU-17 was designed to introduce starting ATG codon just prior to the N-terminus of mature TS protein, and a NcoI site for subcloning. The PCR product obtained using NU-17 and NU-18 were treated with Nco I and BgI II, and subcloned into the Nco I and Bam HI sites of pET-21d (Novagen, Milwaukee, Wis.). Most of the 12 amino acid repeats and hydrophobic region at the C-terminus were removed by PCR using synthetic DNA primers NU-19 (5′-GTTCCGAACGGTTTGAAGTTTGCG-3′, SEQ ID NO:22) and NU-20 (5′-GTTCCGAACGGTTTGAAGTTTGCG-3′, SEQ ID NO:23). NU-20 corresponds to the partial sequence of the tandem repeat and, in addition, it contains a Sal I site. The PCR product with the minimum numbers of repeats (i.e., 5 repeats) was selected and used to replace the original C-terminus of clone 154 at unique Mlu I site (FIG. 15A). The DNA fragment with unique BamHI/Sal I sites was ligated to the pET-21d plasmid containing the N-terminal region of TS using the Bam HI and Xho I sites of the vector, to yield construct TS-154 (FIG. 4A). To generate construct TS-H32, the Bgl II/Pst I DNA fragment ofpTS-154 was replaced with the corresponding fragment from the gene 121 (Uemura. J. et al., EMBO J. 11:3837-3844, (1992)). The trans-sialidase encoded by gene 121 is catalytically inactive due to a single amino acid difference in the Bgl II/Pst I fragment, with histidine (H374) replacing tyrosine in the catalytically active TS 154 (Uemura. J. et al., EMBO J. 11:3837-3844, (1992)). Thus, construct TS-H32 was inactive (FIG. 15B). All constructs were verified by automated sequencing (ABI Perkin Elmer) using BIGDYE terminator. Production and purification of TS-154 and TS-H32 proteins was identical to the method described above for the TS-F construct.

### Immunoassays for Cytokines

Endothelial cells and T-24 cells were plated on 24-well plates at a density of 1×10³ cells/well, while peripheral blood mononuclear cells (PBMC) were plated in the similar wells at 1×10⁶/well. Triplicat cultures of cells were incubated test agents for a predetermined amount of time. Polymixin B was used at 10 μg/ml in all cell cultures, as it did not affect any parameter tested. Cytokines and chemokines released in the culture supernatants were measured by ELISA assay following the instructions of the manufacturer (Endogen). The cytokines tested were IL-1β, IL-4, IL-8, IL-1, IL-12, IFN-γ, TNF-α, and the chemokines RANTES and MCP-1. Negative controls were cells incubated in medium containing polymixin B at 10 μg/ml, and positive controls were cells incubated with bacterial LPS at the low ng/ml range (for PBMC or HIMEC) or in the μg/ml range for carcinoma T-24 cells. In some experiments, IL-1β or TNFα were used as positive control for cytokine release.

### Bioassay for IL-6

Bioassay for IL-6 was performed using IL-6 dependent human DS-1 cells (Bock et al., Cytokine 5: 480-489 (1993), ATCC Accession No. CRL-11102). Briefly, 1×10⁴ cells/well were plated in 96-well plates and incubated for 24 hours in IL-6-free medium (10% FCS in RPMI) containing several dilutions of TS-conditioned media or exogenous rIL-6. The cultures were pulsed with 0.5 μCi ³H-thymidine for 4 hours and harvested to determine radioactivity incorporation using a microplate scintillation instrument (Packard). In some studies, a neutralizing anti-IL-6 rabbit IgG or normal rabbit IgG (Endogen) was added to the dilutions of TS-conditioned prior to assaying for growth-stimulation of DS-1 cells. TS conditioned media was prepared by incubating PBMC or T-24 cells for 24 hours in 10% FCS/RPMI without (CM) or with (TS/CM) TS at 1 μg/ml. Supernatants were centrifuged at 1,000×g to remove cell debris and were kept frozen at −20° C. until use.

Polymerase Chain Reaction of Reverse-Transcribed mRNA

Semiquantitative analysis of cytokine mRNA was performed by the primer-dropping method (Wong et al., Anal. Biochem. 223:251-258, (1996)). RNA of endothelial cells (1×10⁵) or PBMC (1×10⁶), that were or were not stimulated with TS at 1 μg/ml for 24 hours, was purified by acid guanidinium isothiocyanate-phenol-chloroform-TR1 reagent (Molecular Research Center). Two μg of total RNA were converted to cDNA in a volume of 20 μl using random hexamer primers according to the manufacturer's instructions (GIBCO-BRL). Synthetic DNA primers for human IL-6 were 5′ATGAACTCCTTCTCCACAAGCGC (SEQ ID NO:24) and 5′GAAGAGCCCTCAGGCTGGACTG (SEQ ID NO:25), and for GAPDH were those described in Example 1 (SEQ ID NO:10 and SEQ ID NO:11). The PCR mixture contained 20 mM Tris-HCl pH 8.4, 50 mM KCl, 1.5 mM MgCl₂, 1 mM dNTP, 200 pM primers and 1 U of Taq polymerase in 50 μl. Thermal cycling conditions were: denaturation step 95 degrees for 5 min; 35 cycles of 95 degrees 30 sec, 60 degrees 30 sec and 75 degrees 1 min, and a final step of 75 degrees for 5 min. At cycle, number 9, GAPDH primers were added. Five microliters of the PCR product were alalyzed by electrophoresis through 2% agarose-gel in the presence of 1 μg/ml ethidium bromide.

### LTR Depletion

Two hundred microliters of Sepharose-G protein (Pharmacia) were adsorbed with culture supernatants of the mAb TCN (IgG1 isotype) or of a control mAb IgG1 specific for p-azo-phenylarsonate (kindly provided by Dr. Thereza Imanishi-Kari, Tufts Medical School, Boston, Mass.), washed with 20 vols of LPS-free PBS, pH 7.2. Three micrograms of LTR were loaded to either protein G/TCN-2 or protein G/control IgG1 column. The effluent was reapplied 5 times to the respective column. The last flow-through of each column was collected in 200 μl in a separate LPS-free tube, and the columns were washed with five column volumes of PBS. Elution of bound LTR was by mixing the resins with a spatula and centrifugation at 250×g for 5 min at 4° C. Effluents and eluates were constituted to the original volume and added to T-24 cells. After 24 hours, IL-6 in the T-24 cell supernatants were determined by ELISA.

IL-6 Production by PBMC Stimulated with TR Peptides

TR peptides (Table 2) were synthesized at the Tufts Synthesis Facility (Boston, Mass.). Each peptide was purified by HPLC, and the number of amino acids and molecular weight of each peptide verified by mass spectrometry. Peptides were dissolved in growth medium (10% FCS in RPMI) and added at various concentrations to 1×10⁶ PBMC/well in 24-well plates. After 24 hours, IL-6 was assayed by ELISA in the conditioned supernatants.

TABLE 2
TR peptides

| | |
|---|---|
| Peptide | Amino Acid Sequence |

| | |
|---|---|
| TR1 | DSSAHGAPSTPA |
| | (SEQ ID NO:32) |
| TR2 | DSSAHGAPSTPADSSAHGTPSTPV |
| | (SEQ ID NO:26) |
| TR3 | DSSAHGAPSTPADSSAHGTPSTPVDSSAHGTPSTPA |
| | (SEQ ID NO:27) |
| TR4 | DSSAHGAPSTPADSSAHGTPSTPVDSSAHGTPSTPADSSAHS |
| | TPSTPA |
| | (SEQ ID NO:28) |
| TR5 | DSSAHGAPSTPADSSAHGTPSTPVDSSAHGTPSTPADSSAHS |
| | TPSTPADSSAHSTPSTPA |
| | (SEQ ID NO:29) |

Peptides TR1, TR2, TR3, TR4 and TR5 are derived from the amino acid sequence of the protein encoded by clone 7F (GenBank accession number M61732).

### Results

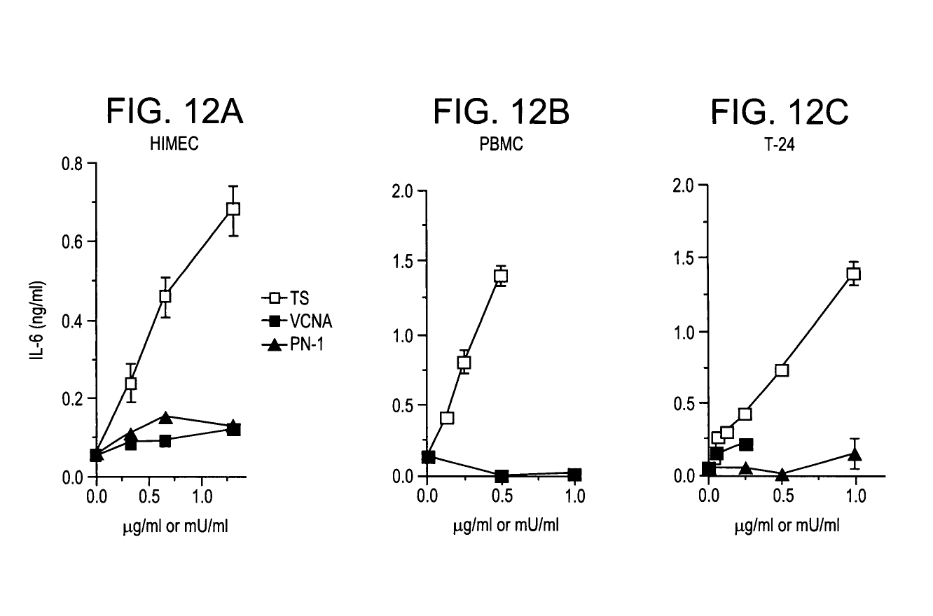

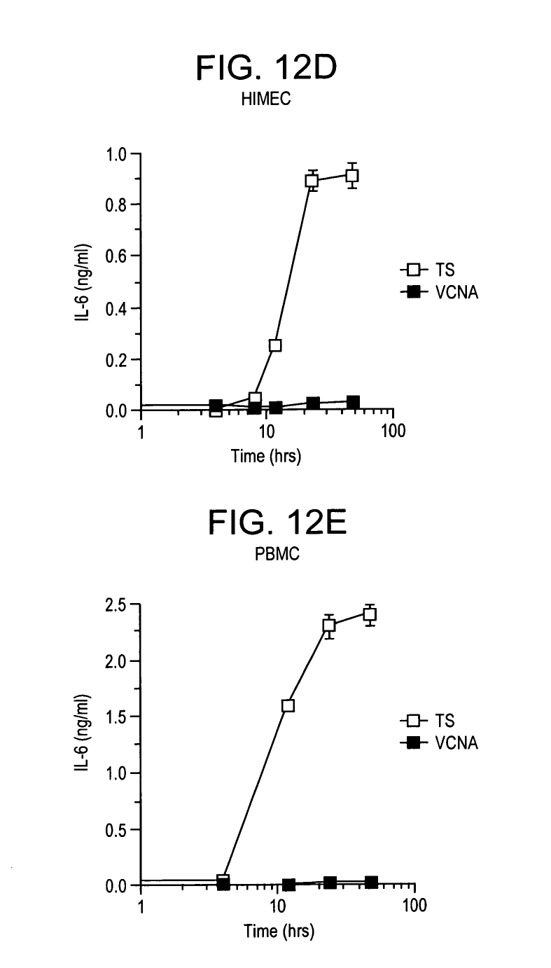

HIMEC monolayers were cultured with various concentrations of purified TS for 24 hours and the concentration of eight cytokines (IL-1β, IL-4, IL-6, IL-8, IL-10, IL-12, INF-γ and TNF-α) and two chemokines (RANTES and MCP-1) in the conditioned HIMEC supernatants was measured. The concentration of IL-6 was elevated in the conditioned supernatants in a manner dependent on the TS input (FIG. 12A). In contrast, the concentration of the other cytokines did not increase in response to TS stimulation under condition in which bacterial LPS at 50 ng/ml released IL-1β, IL-8 and TNF-α. Similar IL-6 upregulation was observed with two other primary cultures of HIMEC. The quantity of IL-6 produced by HIMEC in response to 1 μg/ml of TS corresponded to that induced by 50 ng/ml of a bacterial LPS. A control neuraminidase (VCNA) did not stimulate detectable IL-6 secretion in HIMEC (FIG. 12A) suggesting that the IL-6 secretion inducing action of TS did not depend on its intrinsic glycosidase activity. Another control protein, the T. cruzi heparin-binding penetrin (PN-1) thought to promote parasite invation (Ortega-Barria et al., Cell 67:411-421 (1991)), was not effective in stimulating IL-6 secretion (FIG. 12A), further emphasizing the selectivity of the TS action.

TS also stimulated IL-6 release in normal peripheral blood mononuclear cells (PBMC) (FIG. 12B), another class of human cells relevant to the immunity against T. cruzi. Similar results were obtained with PBMC obtained from three other blood donors. In addition, TS stimulated IL-6 secretion in the human bladder carcinoma T-24 cell line (FIG. 12C). Previous workers shows that T-24 cells express IL-6 constitutively and in response to cytokine stimuli (Bubenik et al., Int. J. Cancer 11:765-773 (1973); Yasukawa et al., EMBO J. 6:2939-2945 (1987)). Interestingly, the IL-6 produced upon 24 hour-stimulation of T-24 carcinoma cells with 1 μg/ml of TS corresponded to that induced by 0.75 μg/ml of bacterial LPS under the same conditions. Thus, in this cancer cell line, TS was nearly as good as bacterial LP in stimulating IL-6 secretion.

IL-6 is produced by a large variety of cells, including endothelial cells, monocytes, fibroblasts, keratinocytes, T cells, mast cells, neutrophils, tumor cell lines, and cells of neural origin (Stein et al., Drug Discovery Today 3:202-213 (1998)). TS stimulates IL-6 production in endothelial cells and PBMC, and it is likely that the neuraminidase will also stimulate IL-6 release in other cell types. However, normal human neutrophils, which are capable of secreting IL-6 in response to some stimuli (Cicco et al., Blood 75:2049-2052 (1990)), did not produce detectable IL-6 when stimulated with TS under condition in which TNF-α did elicit secretion of IL-6.

### Kinetics of TS-Dependent IL-6 Secretion

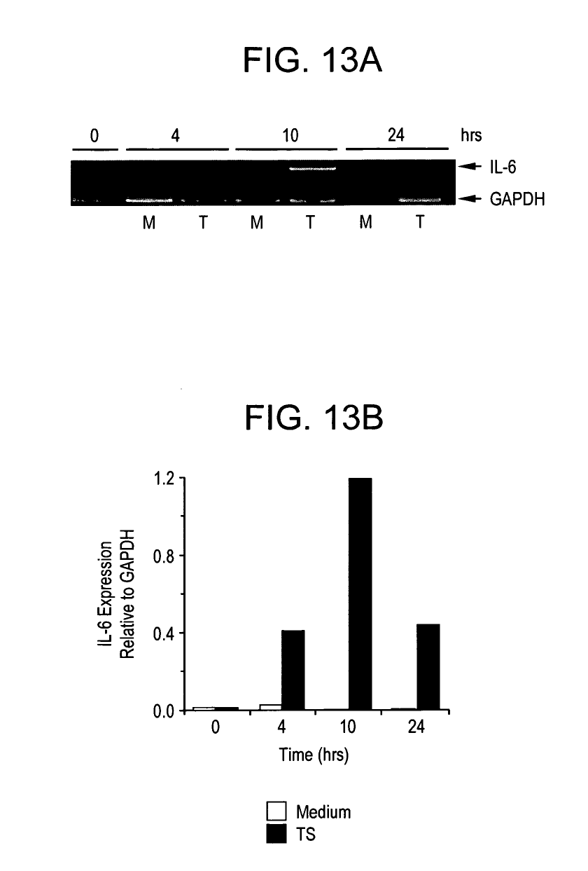

The kinetics of TS-induced IL-6 secretion revealed maximum effect after 24-hour stimulation in both HIMEC and PBMC (FIGS. 12D and 12E). This response proceeded the upregulation of IL-6 transcripts, which was maximal 4-10 hours after TS stimulation (FIG. 13). These results suggest that TS triggers synthesis and then secretion of IL-6, consistent with the effect of conventional cytokine agonists in the same cell types (Nilsen et al., Gut 42:635-642 (1998); Bubenik et al., Int. J. Cancer 11:765-773 (1973)).

### TS-Conditioned Cell Supernatants Restore Growth of an IL-6-Dependent B-Lymphoma Cell Line

The DS-1 B-lymphoma cells have an intact IL-6 receptor signaling pathway, but because they cannot produce IL-6, the cells will die unless exogenous human IL-6 is added to the culture medium (Bock et al., Cytokine 5:480-489 (1993)). This cell line was used to assay conditioned culture media for biologically active IL-6.

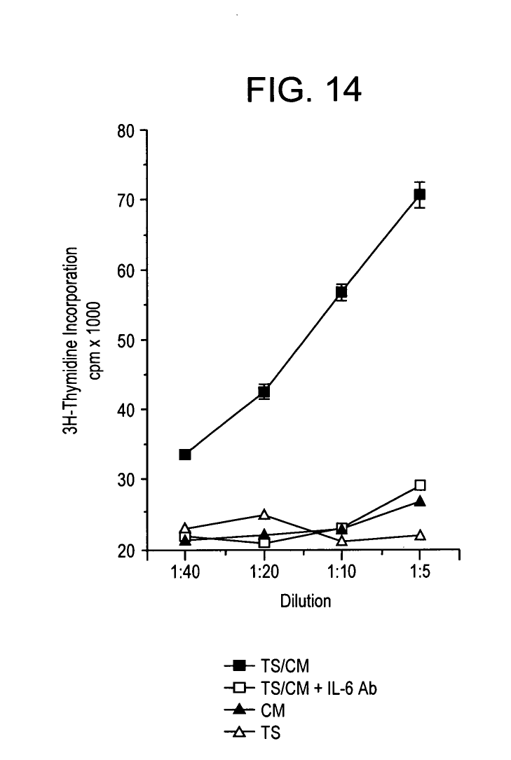

Conditioned media were prepard by incubating PBMC or T-24 cells for 24 hours in 10% FCS/RPMI without (CM) or with (TS/CM) TS at 1 μg/ml. Conditioned media from cells stimulated with TS (TS/CM), but not cells cultured in media along, restored growth of the DS-1 lymphoma cells in a dose-dependent manner, and a neutralizing IL-6 antibody suppressed the growth-promoting activity (FIG. 14). These results demonstrate that IL-6 produced by PBMC or T-24 cells, after culture with TS is biologically active. In addition, TS is not an IL-6 receptor agonist because it did not restore growth of the DS-1 cells when added to normal growth medium lacking exogenous IL-6 (FIG. 14).

### TS Mediates IL-6 Release Through its C-Terminal Tandem Repeat

The IL-6 secretion-inducing activity of the catalytically inactive TS mutant TS-H32 and the catalytically active TS-154 protein were assessed. Both TS-H32 and TS-154 constructs contain 5 tandem repeat units (FIG. 15A). Although TS-H32 differs from TS-154 in six amino acid substitutions in the catalytic domain (FIG. 15A), the difference in enzymatic activity between the two constructs is attributed to a single amino acid, with tyrosine (Y374) in the active enzyme (TS-154) mutated to histidine in the inactive enzyme (TS-H32) (FIGS. 15A and 15B). Surprisingly, enzymatically-active TS-154 was as powerful as enzymatically-inactive TS-H32 in stimulating IL-6 secretion in PBMC (FIG. 15C) and T-24 cells.

The results with the TS-154 and TS-H32 constructs suggest that the catalytic activity of TS does not mediate IL-6 release in naive cells. This was confirmed with the use of recombinant catalytic domain of TS.

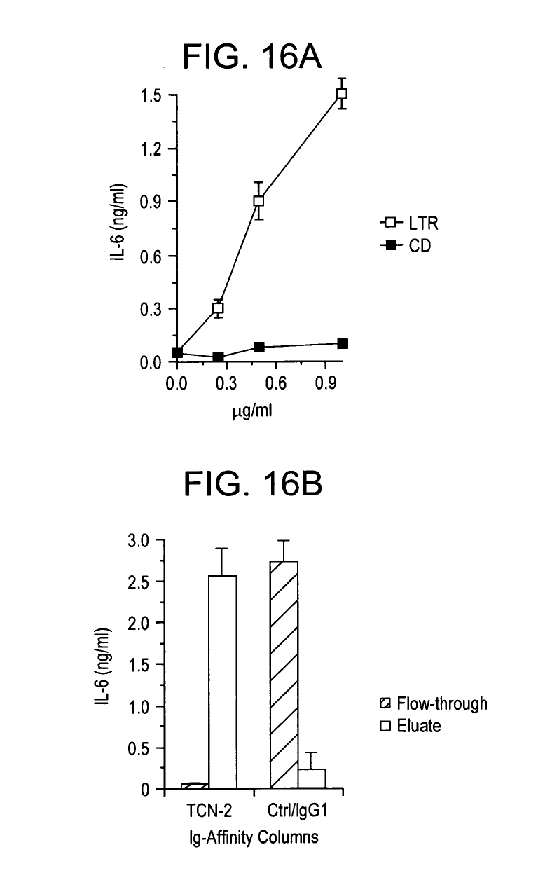

The catalytic domain of TS (CD, also referred to as TS-F) did not promote IL-6 release from T-24 cells, whereas LTR effectively induced release of IL-6 in a dose-dependent manner (FIG. 16A). Similar results were obtained in cultures of PBMC, in which LTR, but not CD, upregulated IL-6 release. To confirm that LTR mediated IL-6 release, we depleted LTR from solution in a protein G-Sepharose column adsorbed with TCN-2. The flow-through of the TCN-2 affinity column, which was devoid of LTR polypeptide as determined by immunoblot analysis did not stimulate IL-6 release in T-24 cell cultures (FIG. 16B). In contrast, the flow-through of a control IgG1 column did contain LTR polypeptide and stimulated IL-6 secretion (FIG. 16B). Furthermore, elution of LTR from the TCN-2/protein G column, restored the IL-6-secretory power of the original preparation, while similar elution from the IgG1 control column did not (FIG. 16B). These results establish that LTR induced IL-6 secretion in naive human cells.

### Synthetic Peptides Based on LTR Induced IL-6 Secretion

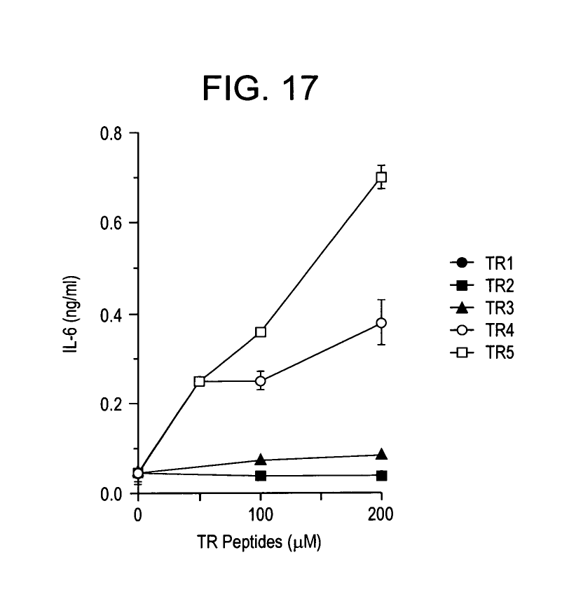

Synthetic peptides TR1 (SEQ ID NO:32), TR2 (SEQ ID NO:26), TR3 (SEQ ID NO:27), TR4 (SEQ ID NO:28) and TR5 (SEQ ID NO:29), which correspond to the 12, 24, 36, 48 and 60 amino acid sequences proximal to the C-terminus of the catalytic domain of TS (T. cruzi, Silvio Stain) (Table 2) (Pereira et al., J. Exp. Med. 174:179-191, (1991)). The IL-6 secretion-inducing activity of the synthetic peptides was assessed in cultures of PBMC. Peptides TR4 (SEQ ID NO:28) and TR5 (SEQ ID NO:29) were active in promoting IL-6 release, while TR1 (SEQ ID NO:32), TR2 (SEQ ID NO:26) and TR3 (SEQ ID NO:27) were less active (FIG. 17). The results with TR peptides confirm that the tandem repeat is the TS moiety that mediates IL-6 release in naive human cells.

HIMEC Strongly Induces IL-6 Secretion When Infected with T. cruzi Trypomastigote Populations Bearing a TS⁺ but not a TS⁻Phenotype

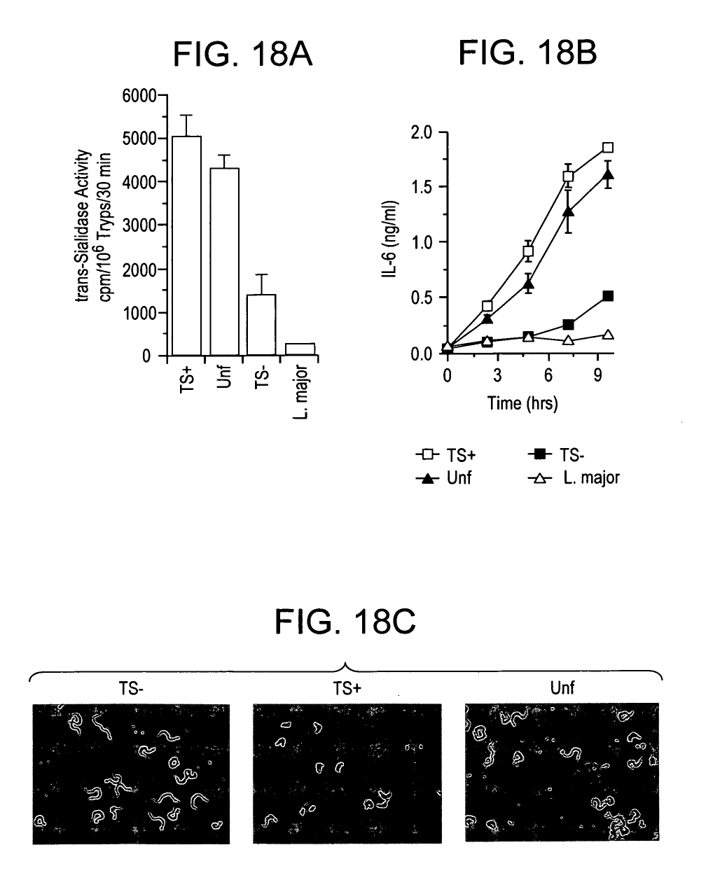

Extracellular trypomastigotes can be subdivided into two populations based upon the relative abundance of TS (Pereira, M. E. A. et al., Infect. Immun., 64:3884-3892 (1996)). These two populations can be readily separated from one another by differential affinity to magnetic beads coated with LTR-specific mAb TCN-2. The subset that produces TS (TS⁺) represents about 25% of the total trypomastigote population, while the subset that does not produce or produces very little TS (TS⁻) constitutes the majority of trypomastigotes (Pereira, M. E. A. et al., Infect. Immun., 64:3884-3892 (1996)). TS⁺ parasites of the Silvo strain are short and stumpy (length=9.3±2.8 μm) and morphologically distinct from TS⁻ parasites, which are slender (length=18.2±4.3 μm) (FIG. 18C). Live TS⁺ trypomastigotes moved slowly and sluggishly in liquid medium (RPMI/10% FCS) at room temperature. In contrast, TS⁻ trypomastigotes migrated through liquid media swiftly with a whipping movement. However, the dimorphism and contrasting movements of TS⁺ and TS⁻ trypomastigotes were not characteristic features of two other T. cruzi strains, Tulahuen and MV-13. Thus, there does not appear to be a relation between the expression of TS and a particular morphological type of T. cruzi.

Nevertheless, the availability of purified TS⁺ and TS⁻ populations offered a unique opportunity to test the capacity of live parasites with variable abundance of TS to induce IL-6 secretion by normal human cells. HIMEC monolayers were challenged with live TS⁺ and TS⁻ parasites and the secretion of IL-6 was assessed over time. TS+trypomastigotes were much more effective than TS⁻, and somewhat better than unfractionated trypomastigotes, in inducing release of IL-6 (FIG. 18B). The results presented in FIG. 18B are from studies using the Silvio strain. Similar results were obtained in studies using the Tulahuen strain. Because the TS⁺ parasites of the Tulahuen strain are a mixture of stumpy and slender forms, the power of T. cruzi to induce IL-6 secretion in naive human cells is dependent on the expression level of TS and not on the morphology of the parasite. Furthermore, Leishmania major promastigotes which do not have TS activity (FIG. 18A) do not induce IL-6 secretion in HIMEC (FIG. 18B). Thus, parasite burden per se does not suffice to induce IL-6 secretion in naive human cells.

### Discussion

Infection of mammals by parasites and other microbes results in the release of cytokines and other mediators of the inflammatory response. The composition of the cytokines, which depends on the nature of the infecting oranism and on the host genotype, may be critical for the resistance or susceptibility to microbial invation, as best exemplified by the infection of mice with the protozoan Leishmania major (Reiner et al., Annu. Rev. Immunol. 13:151-177 (1995)) and of humans with the bacterium Mycobacterium leprae (Yamamura et al., J. Immunol. 149:1470-1475 (1992)).

It is generally accepted that cytokine networks result from antigenic stimulation of lymphocytes and macrophages. However, these antigen-driven host responses can be subverted by a group of viral and bacterial proteins, termed virokines and bacteriokines, which are capable of changing the dynamics of the cytokine networks without directly activating B and T cell receptors (Wilson et al., Infect. Immun. 66:2401-2409 (1998)). Virokines tend to suppress host immune responses by neutralizing inflammatory cytokines, as is the case of the protein B15R of vaccinia virus, which binds IL-1β (Alcami et al., Cell 71:153-167 (1992)). Alternatively, virokines may down-modulate immune responses by mimicking anti-inflammatory cytokines, like the protein BCRF1 of Epstein-Barr virus, which is 70% identical to IL-10 (Hsu et al., Science 250:830-832 (1990)). On the other hand, bacteriokines, including lipopolysaccharides (LPS) and exotoxins, are more likely to stimulate proinflammatory cytokines, thereby enhancing pathogenesis (Wilson et al., Infect. Immun. 66:2401-2409 (1998)). Whether protozoan parasites can alter host immune responses through molecules functionally equivalent to virokines and bacteriokines (i.e., ‘protokines’) remains to be determined. The findings described herein identify TS as a protein that can alter the dynamics of the cytokine network by upregulating IL-6 secretion in normal human cells. As described herein, TS and its tandem repeat induced secretion of IL-6 by naive microvascular endothelial cells and PBMC. Furthermore, given that IL-6 may be produced by many other cell types, such as fibroblasts and epithelial cells, the range of target cells for IL-6 release by TS is broader than the vascular endothelium and blood mononuclear cells. This was indicated by the ability of TS to induce IL-6 release in the T-24 bladder carcinoma cells (FIG. 12C) and by parallel experiments with mouse cells, which revealed TS to be an upregulator of IL-6 secretion in naive splenocytes, bone marrow cells, and peritoneal cells from BALB/c and other strains of mice. TS, therefore, upregulates IL-6 secretion in various cell types and mammalian species.

The kinetics of TS-dependent IL-6 release in vitro (FIGS. 12D, 12E and 13) suggests that, in vivo, TS can stimulate IL-6 release prior to the full development of acquired immune response against T. cruzi. This could be accomplished when T. cruzi first encounters the mammalian host, such as after an insect bite, when the parasites enter the host through the mucosa, usually around the eye. Or when T. cruzi enter the host during blood transfusion or congenitally, in which case the TS parasites gain access into the circulation, where they can react with endothelial cells and PBMC to trigger IL-6 release. In addition, TS can also promote IL-6 secretion in vivo as a soluble mediator. Indeed, soluble neuraminidase was detected, before the parasites, in the blood of a T. cruzi-infected individual (DeTitto et al., Clin. Immunol. Immunopathol. 46:151-157 (1988)). Copious amounts of the enzyme are released in monolayers of cells infected with T. cruzi (Scudder, P. et al., J. Biol. Chem., 268:9886-9891 (1993)). The findings reported herein further show that soluble enzyme and TS-expressing parasites are capable of inducing IL-6 secretion in normal cells.

There are several ways in which TS-dependent IL-6 could alter T. cruzi invasion. IL-6 promotes polyclonal activation of B and T lymphocytes (Kishimoto, T., Blood 74:1-10 (1989); Uyttenhove et al., J. Exp. Med. 167:1417-1427 (1988)) and is an important cofactor for Th2 T cell activation, necessary for the induction of humoral immune responses (Rincon et al., J. Exp. Med. 185:461-469 (1997)). Thus, the TS/IL-6 pathway could be directly relevant to the polyclonal lymphocyte responses that characterize acute Chagas' disease (Minoprio et al., J. Immunol. 24:661-668 (1986)). In addition, IL-6 is a potent inducer of collagen secretion in human fibroblasts (Duncan et al., J. Invest. Dermatol. 97:686-692 (1991)), and as such appears to underlie the pathogenesis of systemic sclerosis, a connective tissue disease characterized by fibrosis in the skin and internal organs (Kawaguchi et al., J. Clin. Invest. 103:1253-1260 (1999)). The fibrogenic action of IL-6 could also be relevant to T. cruzi infection, as fibrosis is a prominent feature of acute and chronic Chagas' disease (Köberle, F., In Ciba Found Symp 20, pp 137-147, 1974; Andrade, Z. A. In Ciba Found. Symp. 99:214-233 (1983)). Because chronic chagasic heart contains many IL-6-containing mononuclear cells and endothelial cells (Chandrasekar et al., Biochem. Biophys. Res. Commun. 233:365-371 (1996); Zhang et al., Exp. Parasitol. 84:203-213 (1996)), IL-6 could contribute to the fibrosis in the chronic heart, regardless of the mechanism stimulating production of the cytokine.

IL-6, in addition to mediating inflammatory and immune responses, can play an important role in a variety of central and peripheral nervous systems, such as cell-to-cell signaling, protection of neurons from insult, as well as neuronal growth and survival (Gruol et al., Mol. Neurobiol. 15:307-339 (1997)). Thus, TS-dependent IL-6 can be a factor in the neuroregeneration that characterizes the indeterminate phase of Chagas' disease in humans (Köberle, F. In Ciba Found. Symp. 20: pp. 137-147 (1974)) and animals (Tafuri, W. L., Am. J. Trop. Med. Hyg 19:405-417 (1970)).

As described herein, TS is extremely potent in protecting several types of neuronal cells from undergoing apoptosis induced by growth factor deprivation. Furthermore, the TS-induced neuroprotection was syngergistic with two members of the IL-6 family, ciliary neurotrophic factor (CNTF) and leukemia inhibitory factor (LIF) (See Example 1). Thus, the neurotrophic effect of TS-dependent IL-6 could be boosted by the action of TS and by the TS synergy with CNTF or LIF on the neurons.

### EXAMPLE 4

### Peptide Y21 (SEQ ID NO:15, Also Referred to as Peptide C19Y21) Promotes Survival and Differentiation of Neuronal Cells Through TrkA Nerve Growth Factor Receptor

The human parasite Trypanosoma cruzi, the agent of Chagas' disease, expresses a membrane-bound and shed neuraminidase/trans-sialidase, also referred to as TS or as parasite-derived neurotrophic factor (PDNF) because it binds and activates high affinity nerve growth factor (NGF) receptor TrkA in neuronal cells. Here we identify a small stretch of 21 amino-acids (⁴²⁵GNASQNVWEDAYRCVNASTAN⁴⁴⁵; SEQ ID NO:15) on the surface of the lectin-like domain of PDNF that reproduces neurotrophic activities of intact PDNF.

Synthetic peptide Y₂₁ (SEQ ID NO:15), modeled on the amino-acid stretch, promotes survival and neurite outgrowth in TrkA-expressing PC12 cells but not in TrkA-deficient PC12^{nnr5} cells. Other synthetic peptides (truncated variants of Y₂₁, PDNF-based peptides BR (also referred to as peptide B2; SEQ ID NO:18) and TR (SEQ ID NO:19)) were all inactive in promoting PC12 cell survival and differentiation. Macromolecularization of Y₂₁ (SEQ ID NO:15), but not of TR (SEQ ID NO:19), turned otherwise inactive bovine serum albumin into a promoter of PC12 cell survival. Peptide Y₂₁ (SEQ ID NO:15), but not the other synthetic peptides, enhanced phosphorylation of TrkA in PC12 cells and promoted activation of Erk and Akt kinases with a kinetics distinct from that of PDNF. In addition, peptide Y₂₁ (SEQ ID NO:15) stimulated regulated phosphorylation of CREB (cyclic adenosine monophosphate response element-binding protein), a transcription factor that regulates genes that respond to NGF. Synthetic peptide Y₂₁ (SEQ ID NO:15), therefore, reproduced several TrkA-dependent neurotrophic properties of PDNF and nerve growth factor (NGF). However, peptide Y₂₁ (SEQ ID NO:15) inhibited the binding of PDNF, but not of NGF, to TrkA. Similarly, peptide Y₂₁ (SEQ ID NO:15) blocked PDNF-, but not NGF-dependent activation of MAPK (mitogen-activated protein kinase) signaling pathway. These findings indicate that peptide Y₂₁ (SEQ ID NO:15) reacts with a TrkA site required for the binding of PDNF, but not of NGF. The functioning of peptide Y₂₁ (SEQ ID NO:15) as a PDNF agonist of TrkA-dependent biological activities will be useful for elucidating the mechanism of PDNF activation of TrkA-bearing cells, and for the design of therapeutics (e.g., small molecule drugs) for the treatment of Chagas' and other neurodegenerative diseases.

### Introduction

Trypanosoma cruzi causes Chagas' disease, a chronic debilitating condition afflicting millions of people in the Americas. T. cruzi can seriously damage and destroy the tissues it infects, including the nervous system, either directly through parasite load or indirectly by eliciting a strong inflammatory response. It is therefore surprising that most patients in acute Chagas' disease, which is characterized by thriving parasites in central and peripheral organs, rarely present symptoms and/or physical signs of brain infection, even when trypanosomes inhabit the cerebrospinal fluid (Hoff et al., N. Engl. J. Med. 298:604-606 (1978)). Furthermore, patients who die with acute Chagas' disease usually reveal morphologically preserved neurons located within foci of inflammatory cells or adjacent to T. cruzi nests (Said et al., Ann. Neurol. 18:676-683 (1985). Like humans, animals acutely infected with T. cruzi can display similar evidence of neuroprotection (Id.), as do patients who progress from acute to chronic asymptomatic (indeterminate) disease, where neuron counts in both GI and heart ganglia can increase with the age of patients in a trend counter to the age-related physiological reduction in ganglia in normal, non-Chagasic individuals (Koberle, Adv. Parasitol. 6:63-116 (1968)). Thus, the pathogenesis and time-course of Chagas' disease suggest the existence of neuroregenerative events.

One explanation for these events is that T. cruzi, promotes healing of the nervous system by secreting and/or stimulating the host to secrete neurotrophic factors to help repair infected and uninfected cells damaged by the invasive process. This explanation is supported by the discovery that trypomastigote, the invasive form of T. cruzi, expresses a GPI-anchored PDNF that directly promotes survival of serum-deprived neuroblastoma cells, primary culture of neurons, and Schwann cells (Chuenkova and Pereira, Mol. Biol. Cell 11:1487-1498 (2000); Chuenkova et al., Proc. Natl. Acad. Sci. USA 98:9936-9941 (2001)). PDNF, originally discovered for its ability to release sialic acid from glycoconjugates into aqueous solution (neuraminidase) (Pereira, Science 219:1444-1446 (1983)) and to transfer sialic acid to β-galactosyl acceptors (sialyl transferase) (Parodi et al., EMBO J. 11:1705-1710 (1992); Schenkman et al., J. Exp. Med. 175:567-575 (1992); Scudder, P. et al., J. Biol. Chem., 268:9886-9891 (1993)), promotes neuronal and glial cell survival independent of sialic acid-binding activities (Chuenkova and Pereira, Mol. Biol. Cell 11:1487-1498 (2000)). In addition, PDNF can stimulate human intestinal microvascular endothelial cells and peripheral blood mononuclear cells to secrete biologically active interleukin-6 (IL-6), which can trigger survival mechanisms in neurons and other cells (Saavedra et al., J. Exp. Med. 190:1825-1836 (1999)). In addition, PDNF has been reported to cause apoptosis in some organs of the immune system (Leguizamon et al., J. Infect. Dis. 180:1398-1402 (1999)), an action consistent with its functional mimicry of host survival factors, which can cause cell death under certain circumstances (Casaccia-Bonnefil et al., Cell Death Differ. 5:357-364 (1998)).

PDNF-induced survival requires activation of PI3K (phosphatidylinositol 3-kinase)/Akt kinase signaling pathway and upregulation of anti-apoptotic bcl-2 gene (Chuenkova and Pereira, Mol. Biol. Cell 11:1487-1498 (2000); Chuenkova et al., Proc. Natl. Acad. Sci. USA 98:9936-9941 (2001)), whereas PDNF-induced neuronal differentiation depends on the activation of the Ras-dependent MAPK/Erk (mitogen-activated protein kinase/extracellular signal-regulated kinase) signaling (Chuenkova and Pereira, Neuroreport 12:3715-3718 (2001)). PDNF exploits MAPK/Erk signaling to inhibit caspase activation, free radical formation, and death of dopaminergic cells exposed to the Parkinsonism-inducing neurotoxin 1-methyl-4-phenylpyridinium (MPP⁺) (Chuenkova and Pereira, Mol. Brain. Res. 119:50-61 (2003)). PDNF binds and activates the NGF receptor TrkA to trigger PI3K/Akt and MAPK/Erk signalings in several types of neuronal cells (Chuenkova and PereiraPerrin J. Neurochem., 91:385-394 (2004); Woronowicz et al., Glycobiology 14:987-98 (2004)).

The ability of PDNF to promote survival and differentiation of neurons have been mapped to its N-terminal region of 632 amino acids (Chuenkova and Pereira, Mol. Biol. Cell 11: 1487-1498 (2000); Chuenkova et al., Proc. Natl. Acad. Sci. USA 98:9936-9941 (2001)), made-up of one domain bearing the neuraminidase/sialyltransferase catalytic sites, and another one having a lectin-like structure (Chuenkova and Pereira, Mol. Biol. Cell 11: 1487-1498 (2000); Chuenkova et al., Proc. Natl. Acad. Sci. USA 98:9936-9941 (2001); Buschiazzo et al., Mol. Cell. 10:757-768 (2002)). In contrast, PDNF-induced stimulation of IL-6 secretion and lymphocyte activation has been assigned to its C-terminal region (Saavedra et al., J. Exp. Med. 190:1825-1836 (1999); Gao et al., Int. Immunol. 14:299-308 (2002)), composed of a long tandem repeat of a 12 amino acid unit (peptide TR; SEQ ID NO:19) (Pereira et al., J. Exp. Med. 174:179-191 (1991)). A peptide mapping approach revealed that a bacterially-expressed truncated PDNF corresponding to amino acids 1-445 promoted survival and differentiation of TrkA-positive PC12 cells while truncated PDNF equivalent to amino acids 1-425 did not (Chuenkova and Pereira, Mol. Biol. Cell 11:1487-1498 (2000)). This finding raised the possibility that the amino acid sequence matching PDNF segment 425-445 reproduces TrkA-dependent neurotrophic properties of the trypanosomal protein.

As shown herein, synthetic peptide Y₂₁ (SEQ ID NO:15), a 21 amino acid peptide corresponding to PDNF amino acid sequence 425-445, is a unique protozoan parasite-derived peptidomimetic agonist of PDNF on TrkA-bearing PC12 cells. Peptide Y₂₁ (SEQ ID NO:15) and the results described herein provide a novel therapeutic strategy to selectively target neuronal and other TrkA-bearing cells.

### Materials and Methods

PC12^{nnr5} and PC12^{trkA} cells were a gift from Dr. L. Greene (College of Physicians and Surgeons, Columbia University, N.Y., USA). Anti-TrkA antibody was purchased from ABCAM (Cambridge, Mass.), while antibodies against phospho-TrkA (Tyr 490), phospho-p42/44 MAPK, phospho-Akt (Ser473), phospho CREB, p42/44MAPK, Akt, and CREB were from Cell Signaling Technology (Beverly, Mass.). HRP-conjugated secondary antibody was from CHEMICON (Temecula, Calif.). ECL kit was from PERKINELMER Life Sciences (Boston, Mass.). Protein G-Sepharose was purchased from AMERSHAM BIOSCIENCES (Piscataway, N.J.). Anti-protease cocktail was obtained from ROCHE MOLECULAR BIOCHEMICAL (Indianapolis, Ind.). Synthetic peptides were obtained from Tufts University Core Facilities (Tufts University, Boston, Mass.). In this example, PDNF refers to the Cys-rich region of 632 amino-acids of clone 19Y (SEQ ID NO:2) composed of catalytic and lectin-like domains but without the C-terminal tandem repeat (Chuenkova et al., Biochem Biophys Res Commun 262:549-556 (1999)); recombinant PDNF was expressed in Escherichia coli and purified by Ni-chelate chromatography and FPLC, as described earlier (Chuenkova et al., Biochem. Biophys. Res. Commun. 262:549-556 (1999)); Chuenkova and Pereira, Mol. Biol. Cell 11:1487-1498 (2000)).

### Bioassays for Neurotrophic Activity

PC12^{nnr5} and PC12^{TrkA} cells were grown in RPMI with 10% FCS and 5% horse serum as described earlier (Green et al., J. Cell Biol. 102:830-843 (1986); Chuenkova and Pereira, Mol. Biol. Cell 11: 1487-1498 (2000)). Two approaches were used to study neurite extension: 1) Plating cells (1×10⁵ cells/ml) on plastic wells (LABTECH 16-well slides, Nalge Nunc International, Naperville, Ill.) coated with synthetic peptides (100 μg/ml water, 4° C., overnight) or rat tail collagen (1 mg/ml PBS, 4° C., overnight). 2) Adding peptides in solution to cells plated on uncoated substratum. Cells were grown in 1% FCS-containing RPMI for 72 hours, and reacted with 0.5 mg/ml MTT (3-[4,5-dimethylthiazol-2-yl]-2,5-diphenyltetrazolium bromide) (SIGMA, St. Louis, Mo.) in serum-free medium for 1 hour at 37° C. Light microscopy was used to visualize viable cells (i.e., containing reduced insoluble formazan) with or without extended neurites (>5 μm in length). Cell viability was also determined using the CELLTITER 96 assay (Promega, Madison, Wis.) as follows: Cells, plated at 2×10⁵/ml, were treated with peptides in serum-free medium for different times and exposed to CELLTITER 96 Aqueous One Solution for 1 hour at 37° C. 2) Apoptotic cells were identified by staining with DAPI (250 ng/ml) (SIGMA) 48 hours after cells were exposed to serum-free medium with or without additives in solution, as described earlier (Chuenkova and Pereira, Mol. Biol. Cell 11:1487-1498 (2000)).

### Peptides Cross-Linking to BSA

Peptides were macromolecularized by cross-linking to bovine serum albumin (BSA) with sulfosuccinimidyl 4-(N-maleimidomethyl)cyclohexane-1-carboxylate, sulfo-SMCC(PIERCE, Rockford, Ill.) according to manufacturers protocol. Briefly, peptides were reduced using 2-mercaptoethylamine to create free sulfhydryl groups, at the same time BSA was activated with sulfo-SMCC. Conjugation was performed by mixing the reduced peptide and activated BSA followed by incubation for 2 hours at 4° C. Conjugates were dialyzed against PBS, sterilized at 0.22 μm filters and stored at −20° C.

### Western Blotting and Immunoprecipitation

Activation state of TrkA, Erk1/2 and Akt kinases and transcription factor CREB was analyzed by assessing phosphorylation by Western blot with appropriate phospho-specific antibodies. For this, cells were plated in complete medium at a density of 3×10⁵ cells/ml overnight on 6-well plates (BECTON DICKINSON LABWARE, Franklin Lakes, N.J.). The medium was changed to serum-free medium and the cells cultured for an additional 24 hours to reduce background phosphorylation. Then monolayers were stimulated with peptides, PDNF or NGF for indicated times, washed with ice-cold PBS, pH 7.4, and lysed with lysis buffer ((20 mM Tris, pH 7.5, 150 mM NaCl, 1 mM EDTA, 1% Triton X-100, 2.5 mM sodium pyrophosphate, 1 mM glycerophosphate, 1 mM Na₃VO₄, and protease inhibitors leupeptin (10 μg/ml) and PMSF (1 mM)). Lysates were cleared by centrifugation at 14,000 rpm for 30 minutes and protein concentration of supernatants was determined by Bradford assay (BIO-RAD, Richmond, Calif.). For Western blotting, 50 μg of cell lysate were resolved by SDS-PAGE, transferred to nitrocellulose, blocked with 5% non-fat milk, and reacted with polyclonal phospho-p42/44 MAP antibody to detect activated Erk1/2 or polyclonal antibody specific for phospho-Ser-473-Akt to identify activated Akt kinase, or with phospho-Ser133-CREB antibody to detect activated transcription factor CREB. Antibody reaction was visualized by horseradish peroxidase (HRP)-conjugated secondary antibody and enhanced chemiluminescence kit (ECL). Some blots were stripped in appropriate buffer (50 mM Tris HCl, pH 7.8, 2% SDS, 1% β-mercaptoethanol) at 50° C. for 60 minutes and re-probed with p42/44MAPK, Akt or CREB antibody to ensure equal loading of proteins. For immunoprecipitation experiments, cell lysates were pre-adsorbed on protein G-Sepharose for 1 hour at 4° C. and after brief centrifugation supernatants were incubated with anti-TrkA antibody for 24 hours at 4° C. with gentle rocking. Formed immunocomplexes were captured on protein G-Sepharose, eluted with SDS-PAGE sample buffer and analyzed by immunoblotting with antibody against phospho-TrkA receptor.

### Detection of TrkA-TrkA Homodimers

Monolayers of PC12 cells plated in 6-well plates (8×10⁵ cells per well) overnight in complete medium, were transferred to serum-free conditions for 24 hours, washed twice with Krebs/Ringer buffer (137 mM NaCl, 4.7 mM KCl, 2.7 mM CaCl₂ 2H₂O, 1.2 mM KH₂PO₄, 1.2 mM MgSO₄.7H₂O, 10 mM HEPES, pH 7.4, 0.1% BSA, 0.1% glucose) and incubated with peptide Y₂₁ (SEQ ID NO:15) or NGF in the same buffer for 1 hour at 4° C. Cross-linking was performed with membrane impermeable bis(sulfosuccinimidyl)suberate (BS³; PIERCE BIOTECHNOLOGY) for 40 minutes at 4° C. Unreacted BS³ was quenched with 50 mM Tris-HCl, pH 7.4. Monolayers were lysed directly in SDS sample buffer and cell proteins were analyzed by SDS-PAGE, followed by Western blot using polyclonal anti-TrkA antibody and ECL.

### Ligand Blotting Assay

Purified recombinant PDNF (Chuenkova and Pereira, Mol. Biol. Cell 11: 1487-1498 (2000)) was subjected to SDS-PAGE, blotted onto nitrocellulose and blocked in 4% BSA, 0.1% triton X-100 at 4° C. overnight. Strips of nitrocellulose containing immobilized PDNF were incubated with recombinant human TrkA/Fc domain (R&D SYSTEMS, Minneapolis, Minn.) alone or with increasing concentrations of peptide overnight at 4° C. Strips were washed with 0.1% triton X-100 in PBS and probed with anti-human HRP-conjugated antibody (Promega, Madison, Wis.). The intensity of the bands present on the X-ray films after ECL development was quantified by a computer-assisted CS-800 Calibrated Densitometer (BIO-RAD, Hercules, Calif.) using QUALITY ONE PROGRAM, version 4.4.0., software.

### Results

### Synthetic Peptide Y₂₁ (SEQ ID NO:15) Promoted Survival and Differentiation of PC12 Cells Expressing TrkA (PC12^{TrkA}) but not of PC12 Cells Lacking TrkA (PC12^{nnr5})

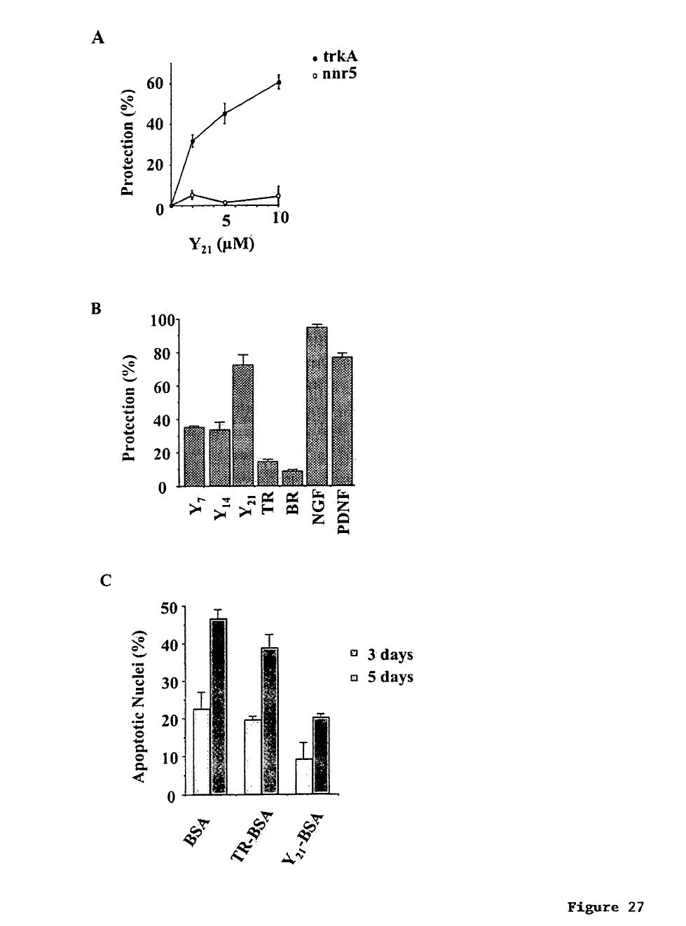

To determine whether peptide Y₂₁ (SEQ ID NO:15) can rescue neuronal cells from apoptosis, PC12^{TrkA} cells were cultured in serum-free medium without and with various concentrations of the synthetic peptide for 3 days, and the proportion of viable cells was determined after staining with the vital dye MTT. The results make clear that peptide Y₂₁ (SEQ ID NO:15) in the low μM range significantly promoted cell survival (FIG. 27A). In contrast peptide Y₁₄ (⁴²⁵GNASQNVWEDAYRC⁴³⁸; SEQ ID NO:35), peptide Y₇ (⁴³⁹VNASTAN⁴⁴⁵; also referred to as peptide CYFN, SEQ ID NO:17), and other PDNF-based peptides BR (⁷⁰YSVDDGETWE⁸⁰; also referred to as peptide B2, SEQ ID NO:18) and peptide TR (⁶³⁹DSSAHGTPSTPA⁶⁵⁰; SEQ ID NO:19) did not significantly promoted cell survival (FIG. 27B). Remarkably, peptide Y₂₁ (SEQ ID NO:15) at 10 μM was nearly as good as 7 nM PDNF or 0.3 nM NGF in promoting survival of PC12^{TrkA} cells (FIG. 27B). Furthermore, covalent coupling of peptide Y₂₁ (SEQ ID NO:15) to BSA turned the otherwise inactive macromolecule (BSA) into a supporter of PC12 cell viability, while similar macromolecularization of synthetic peptide TR (SEQ ID NO:19) did not (FIG. 27C). These results indicate that peptide Y₂, (SEQ ID NO:15) specifically promoted PC12 cell survival.

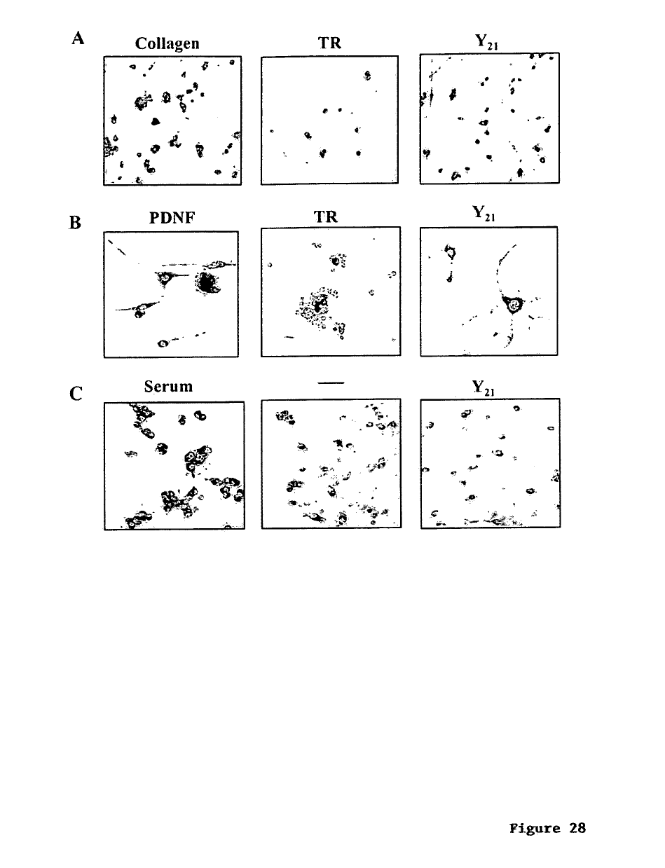

To gain further insights into peptide Y₂₁ (SEQ ID NO:15) mimicry of PDNF, whether the synthetic peptide promotes neurite outgrowth in PC12 cells was assessed. PC12^{TrkA} cells attached, spread, and extended neurites on a substratum coated with peptide Y₂₁ (SEQ ID NO:15), similar to the behavior of these cells on positive control wells coated with collagen (FIG. 28A). In contrast, PC12^{TrkA} cells did not spread and extend neurites on peptide TR-coated (SEQ ID NO:19) substratum, similar to the response of these cells on negative control BSA-coated substratum (FIG. 28A). Further, synthetic peptide Y₂₁ (SEQ ID NO:15) induced neurite extension when added to the medium overlaying PC12^{TrkA} cells that were plated on an uncoated substratum, an effect similar to that of PDNF (FIG. 28B). In contrast, peptide TR (SEQ ID NO:19) did not support survival and neurite extension of PC12^{TrkA} cells when present in the medium overlay (FIG. 28B). Promotion of neurite extension and survival in PC12^{TrkA} cells by peptide Y₂₁ (SEQ ID NO:15) was dependent on TrkA expression, because the peptide was inactive on TrkA-minus PC12^{nnr5} cells (FIG. 28C) (Green et al., J. Cell Biol. 102:830-843 (1986)).

### Synthetic Peptide Y₂₁ Blocked the Binding of the Extracellular Domain of TrkA (TrkA-ECD) to PDNF but not to NGF

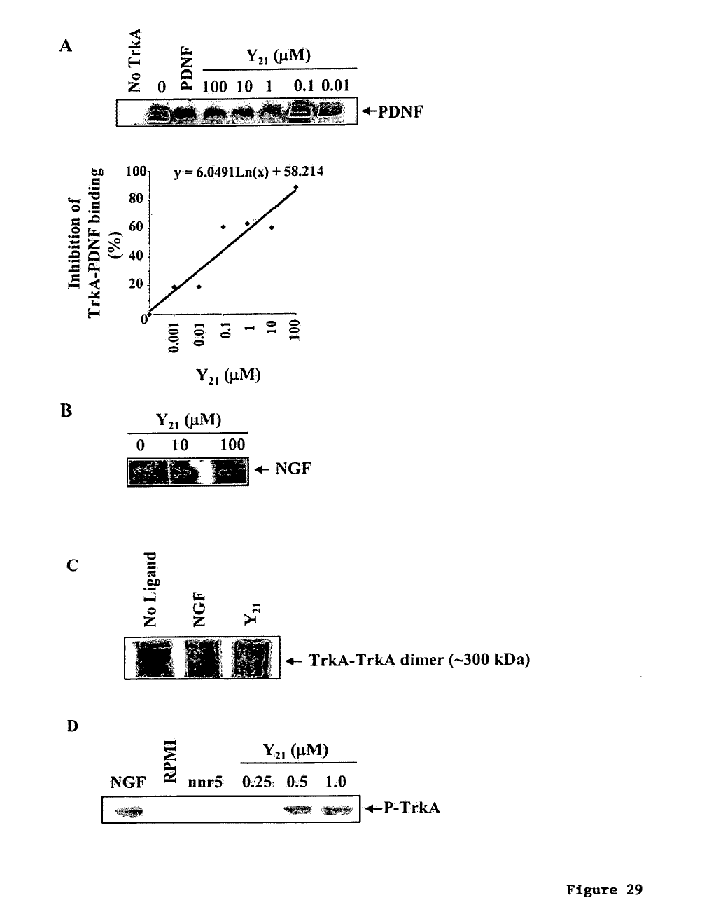

Ligand blotting (Daniel et al., J. Biol. Chem. 258:4606-4611 (1983); Chuenkova and PereiraPerrin, J. Neurochem., 91:385-394 (2004)) was used to gain further insight into the possibility that peptide Y₂₁ (SEQ ID NO:15) interacts with TrkA receptor. Confirming earlier results (Chuenkova and PereiraPerrin, J. Neurochem. 91:385-394 (2004)), soluble extracellular domain of TrkA receptor (TrkA-ECD) bound to nitrocellulose-blotted PDNF [(FIG. 29A, second lane (O)], and the binding was nearly completely inhibited by pre-incubating TrkA-ECD with soluble PDNF (FIG. 29A, third lane, labeled PDNF). When soluble TrkA-ECD was pre-incubated with peptide Y₂₁ (SEQ ID NO:15), the synthetic peptide reproduced the effect of soluble PDNF by competitively inhibiting the reaction of TrkA-ECD with nitrocellulose-blotted PDNF (FIG. 29A). In contrast, binding of TrkA-ECD to NGF was not inhibited by peptide Y₂₁ (SEQ ID NO:15) even at a concentration (100 μM) that blocked >80% binding of TrkA-ECD to PDNF (FIG. 29B).

### Synthetic Peptide Y₂ Activated TrkA Receptor

Incubation of PC 12^{TrkA} cells with peptide Y₂₁ (SEQ ID NO:15) followed by covalent cross-linking of cell surface proteins and immunoblotting of detergent-solubilized cells revealed the generation of a weak TrkA-immunoreactive band of 300 kDa corresponding to TrkA homodimers (FIG. 29C). Because TrkA dimerizes as a prelude to autophosphorylation (Sofroniew et al., Annu. Rev. Neurosci. 24:1217-1281 (2001); Segal, Annu. Rev. Neurosci. 26:299-330 (2003)), this result suggested that peptide Y₂₁ (SEQ ID NO:15) promoted autophosphorylation of TrkA receptors.

To analyze this possibility further, peptide Y₂₁ (SEQ ID NO:15) was added to PC12^{TrkA} cells at 37° C., the cells were lysed with detergent, and the cell lysates were analyzed by immunoblotting with an antibody specific for activated TrkA (phosphorylated at Tyr490). The results showed that peptide Y₂₁ (SEQ ID NO:15) activated TrkA in the low μM range after short-term incubations (5 minutes and 10 minutes) (FIG. 29D), thereby reproducing the effect of PDNF and NGF (Chuenkova and PereiraPerrin, J. Neurochem. 91:385-394 (2004)).

### Synthetic Peptide Y₂, Enhanced MAPK/Erk Signaling

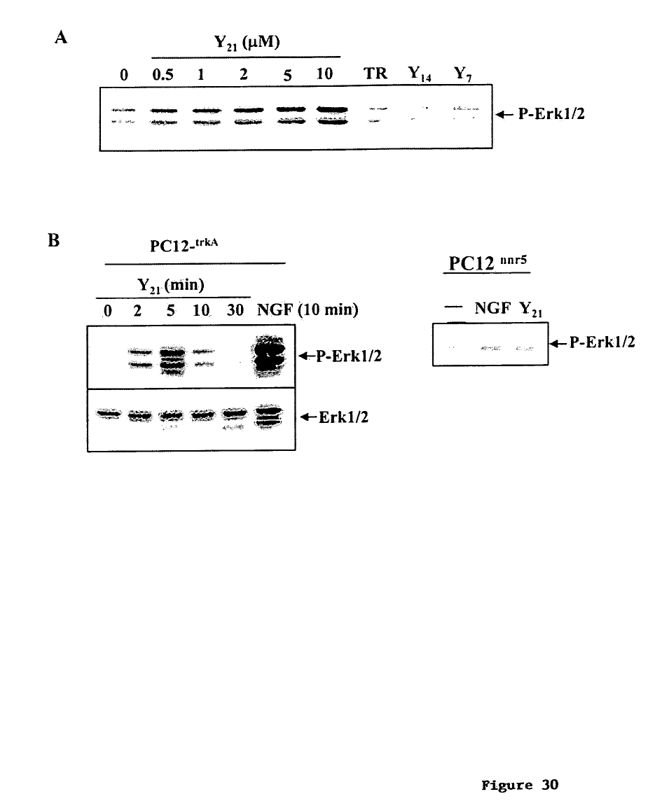

PDNF stimulates neurite extension after activating TrkA and MAPK/Erk signaling (Chuenkova and Pereira, Neuroreport 12:3715-8 (2001); Chuenkova and PereiraPerrin, J. Neurochem. 91:385-394 (2004)). To determine whether peptide Y₂₁ (SEQ ID NO:15) can mimic PDNF by activating MAPK/Erk signaling in a TrkA-dependent manner, a protocol analogous to the one described in for the study in FIG. 29D showing that peptide Y₂₁ (SEQ ID NO:15) induced TrkA activation was used, except that the cell lysates were probed with antibodies specific for unphosphorylated and phosphorylated Erk1/2. The results revealed that peptide Y₂₁ (SEQ ID NO:15) enhanced phosphorylation of Erk1/2 maximally at 5-10 μM (FIG. 30A) and optimally after 5 minutes of stimulation (FIG. 30B, left panel). These kinetics are distinct from that of intact PDNF, which produced a sustained activation lasting ˜30-60 minutes (Chuenkova and Pereira, Neuroreport 12:3715-3718 (2001)). Consistent with the neuroprotection and neurite extension results displayed in FIGS. 27 and 28, synthetic peptides TR (SEQ ID NO:19), Y₁₄ (SEQ ID NO:35) and Y₇ (SEQ ID NO:17) did not activate Erk under the conditions tested (FIG. 30A). Also in line with the conclusion that activation of MAPK/Erk signaling by peptide Y₂₁ (SEQ ID NO:15) is dependent on TrkA expression on PC12 cells, peptide Y₂₁ (SEQ ID NO:15) did not activate Erk1/2 in TrkA-minus PC12^{nnr5} cells, an effect similar to that produced by NGF (FIG. 30B, right panel).

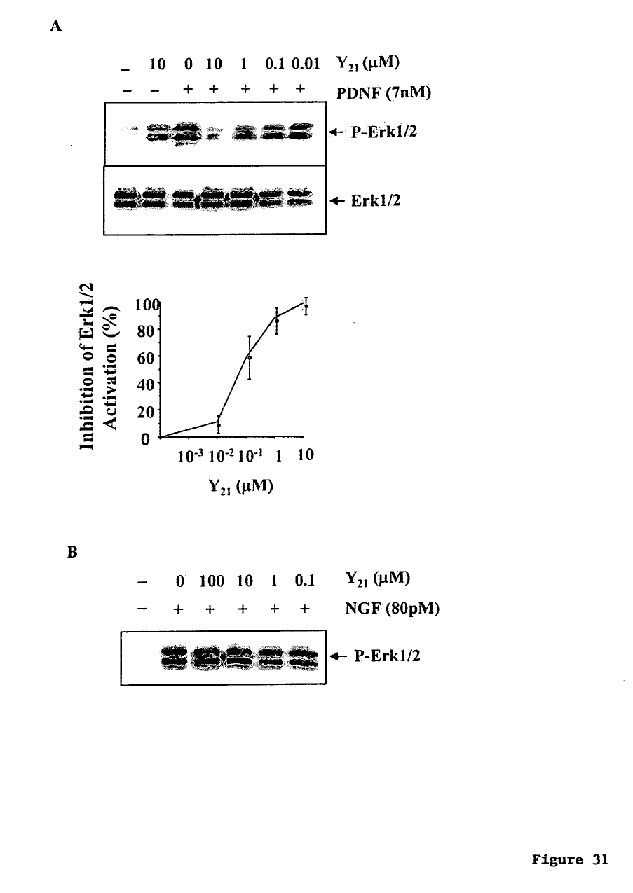

Given that peptide Y₂₁ (SEQ ID NO:15) blocked PDNF binding to TrkA (FIG. 29A) and that PDNF stimulates TrkA-dependent phosphorylation of Erk1/2 (Chuenkova and PereiraPerrin, J. Neurochem. 91:385-394 (2004)), it followed that the synthetic peptide should competitively inhibit PDNF-induced phosphorylation of the MAPK kinase. Indeed, preincubation of PC12 cells with various concentrations of peptide Y₂₁ (SEQ ID NO:15) for 5 minutes followed by a constant activating amount of PDNF (7 nM, 10 minutes) blocked phosphorylation of the Erk1/2 kinase in a dose-dependent manner, and reached nearly 100% inhibition at 10 μM peptide (FIG. 31A). Consistent with the inability to block NGF binding to TrkA-ECD (FIG. 29B), peptide Y₂₁ (SEQ ID NO:15) did not interfere with NGF-induced phosphorylation of Erk kinase (FIG. 31B).

### Synthetic Peptide Y₂, Activated Akt Tyrosine Kinase

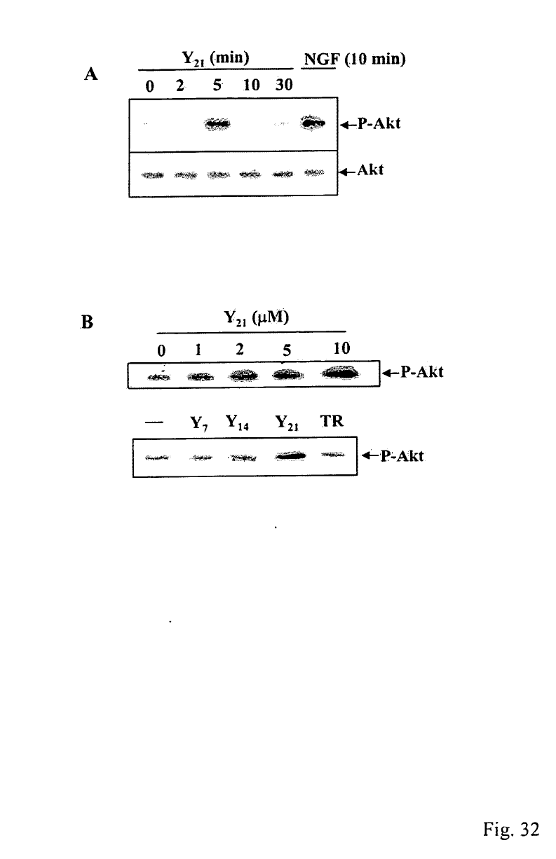

PDNF promotes PC12 cell survival by stimulating PI3K/Akt signaling after binding and activating TrkA receptor tyrosine kinase (Chuenkova and PereiraPerrin, J. Neurochem. 91, 385-394 (2004)). To determine whether the synthetic peptide can also activate PI3K/Akt signaling, PC12^{TrkA} cells were treated with peptide Y₂₁ (SEQ ID NO:15) and phosphorylated Akt kinase (Tyr490) was quantified by immunoblotting. Similar to the stimulation by PDNF and NGF, peptide Y₂₁ (SEQ ID NO:15) activated Akt kinase optimally at 5 minutes (FIG. 32A) and at 10 μM (FIG. 32B, upper panel). PDNF-based synthetic peptides inactive in promoting PC12 cell survival did not activate Akt kinase (FIG. 32B, lower panel).

### Synthetic Peptide Y₂₁ Activated the Transcription Factor CREB

NGF activation of TrkA initiates intracellular signaling cascades that propagate to the nucleus to target transcription factors, in particular CREB (cyclic adenosine monophosphate response element-binding protein). Upon NGF-induced phosphorylation at regulatory site Ser¹³³, CREB activates many genes, including other transcription factors like c-fos (Ginty et al., Cell 77:713-725 (1994); Canon et al., Mol. Biol. Cell 15:5583-5592 (2004)). CREB is a major downstream target of Erk1/2 signaling in the initiation of neuronal differentiation (Ginty et al., Cell 77:713-725 (1994)), and of Akt kinase in the regulation of neuron survival (Du and Montminy, J. Biol. Chem. 273:32377-32379 (1998); Yuan and Yankner, Nature 407:802-809 (2000)).

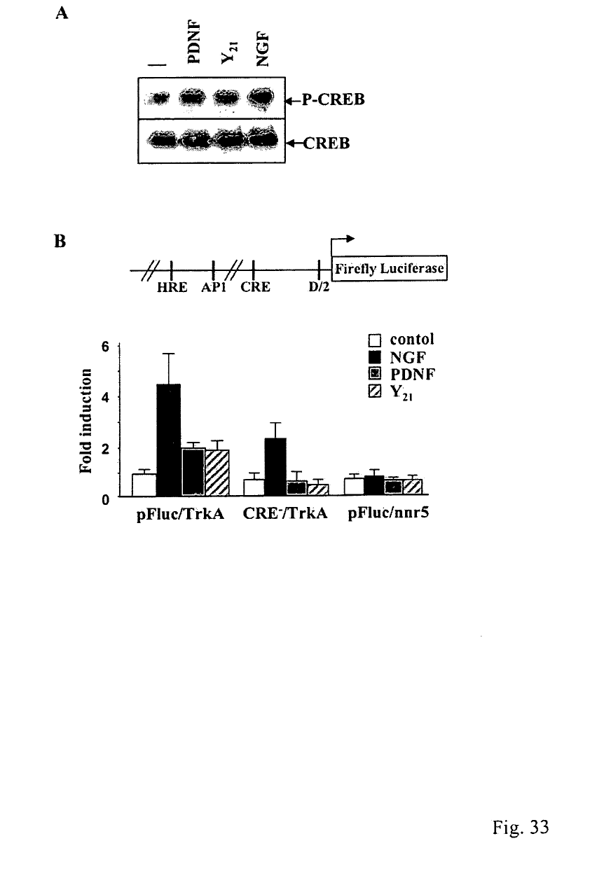

Given that PDNF mimics NGF by promoting TrkA-dependent survival and differentiation of neurons, we sought to determine whether PDNF and PDNF-based synthetic peptide Y₂₁ (SEQ ID NO:15) can activate CREB. Activated CREB was distinguished from non-activated CREB by Western blotting using antibodies that discriminate between Ser¹³³-phosphorylated CREB (P-CREB) and unphosphorylated CREB (Canon et al., Mol. Biol. Cell 15:5583-5592 (2004)). Exposure of PC12^{TrkA} cells to PDNF and peptide Y₂₁ (SEQ ID NO:15) enhanced the formation of phosphorylated CREB, an effect similar to that by positive control NGF (FIG. 33A). This result further confirms, at the transcriptional level, the functional mimicry of NGF by PDNF, and of PDNF by synthetic peptide Y₂₁ (SEQ ID NO:15).

### Discussion

The results presented herein show that synthetic peptide Y₂₁ (SEQ ID NO:15) promoted survival and neurite outgrowth of PC12 cells in a TrkA-dependent manner. Peptide Y₂₁ (SEQ ID NO:15) was biologically active whether bound to the substratum or in solution. The trophic actions of peptide Y₂₁ (SEQ ID NO:15) correlated with its ability to activate TrkA-dependent pro-survival signaling pathways (PI3K and MAPK/Erk) and the transcription factor CREB. In addition to functioning as a peptidomimetic of PDNF, synthetic peptide Y₂₁ (SEQ ID NO:15) competitively inhibited PDNF binding to TrkA and, consequently, PDNF activation of TrkA receptor and PI3K/Akt and MAPK/Erk signaling pathways. These results, therefore, indicated that the amino-acid sequence corresponding to peptide Y₂, (SEQ ID NO:15) forms a site critical for PDNF to bind and activate TrkA receptor and, consequently, enhance survival and differentiation of neurons.

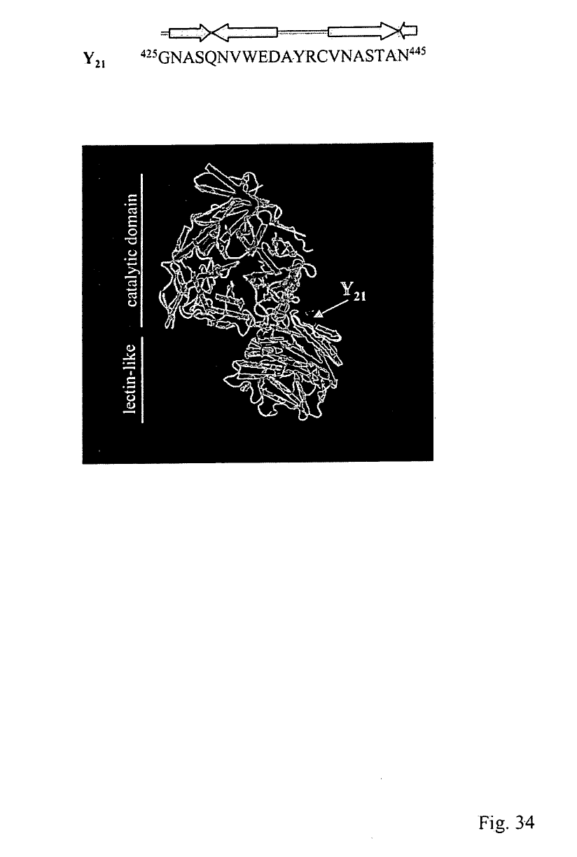

The 3D structure of the N-terminal region of a PDNF isoform reveals a pattern commonly found in other microbial neuraminidases (Gaskell et al., Structure 3:1197-1205 (1995)), one domain with a β-propeller structure containing catalytic sites for neuraminidase and sialyl transferase activities, and an β-helical segment connecting the β-propeller domain to another domain having a β-barrel lectin-like structure (Buschiazzo et al., Mol. Cell. 10:757-768 (2002)), which bears on its surface the peptide Y₂₁ (SEQ ID NO:15) (FIG. 34, lower panel, white arrow). In this context, the peptide Y₂₁ sequence (SEQ ID NO:15) consists of anti-parallel β-strands and a reverse turn and is exposed on the surface (FIG. 34, upper panel). The proposed secondary structure assumes that the Y₂₁ sequence (SEQ ID NO:15) adopts the same conformation as the counterpart sequence of crystallized PDNF, which differ from peptide Y₂₁ (SEQ ID NO:15) in the first six N-terminal amino acids. Such difference is not a surprise because PDNF is highly polymorphic at the protein (Prioli et al., J. Immunol. 144:4384-4391 (1990)) and gene levels (Schenkman et al., J. Exp. Med. 175:567-575 (1994)). In addition, crystals of PDNF were not formed unless seven point mutations were introduced into the molecule (Buschiazzo et al., Mol. Cell. 10:757-768 (2002)). Nevertheless, the presence of a β turn in peptide Y₂₁ (SEQ ID NO:15) is consistent with the segment 425-445 mediating PDNF binding to TrkA receptors, as such loops are almost always exposed on the surface of proteins where they often mediate binding to specific receptor sites, including NGF binding to TrkA (Wiesmann et al., Nature 401:184-188 (1999); Petsko, Protein Structure and Function. New Science press: London, UK (2004)).

The structure of PDNF bears little resemblance to that of NGF, which is a homodimeric molecule formed by twisted β-sheets with a reverse turn in one end, and three reverse turns and cysteine-knot motif in the other end (McDonald et al., Nature 354:411-414 (1991)). NGF binds to membrane-proximal immunoglobulin-like domain in the extracellular portion of TrkA through two patches, patch 1 constituting the binding motif conserved in other neurotrophins, and patch 2 comprising N-terminal residues 2-13, which is specific for NGF/TrkA interaction (Wiesmann et al., Nature 401:184-188 (1999). Several residues in both patches are essential for neurotrophin/Trk receptor interaction, including the NGF C-terminal motif ¹⁰⁹VCV, present in peptide Y₂₁ (SEQ ID NO:15) as CV motif in the transition between reverse turn and β-strand.

Synthetic peptide Y₂₁ (SEQ ID NO:15) competitively inhibited PDNF, but not NGF, binding to TrkA (FIGS. 29A and 29B). Given that PDNF binding to TrkA is inhibitable by NGF, and vice-versa ((Chuenkova and PereiraPerrin, J. Neurochem. 91: 385-394 (2004), it appears that peptide Y21 (SEQ ID NO:15) reacts with a TrkA site dispensable for NGF binding. The extracellular domain of TrkA comprises three leucine-rich motifs, flanked by two cysteine-rich clusters, followed by two Ig-like domains. Although NGF reacts with the membrane-proximal Ig-like motif to activate TrkA (Urfer et al., EMBO J. 14:2795-805 (1995); Robertson et al., Biochem. Biophys. Res. Commun. 282:131-41. (2001)), the other extracellular domains of TrkA also play a role in receptor activation (Huang and Reichardt, Annu. Rev. Biochem. 72:609-642 (2003)). Thus, given the inability of peptide Y₂₁ (SEQ ID NO:15) to inhibit NGF binding to TrkA, presumably to the proximal Ig-like domain, it is possible that peptide Y₂₁ (SEQ ID NO:15), as a synthetic peptide or in the context of PDNF, reacts preferentially with a TrkA domain other than NGF-preferred proximal Ig-like domain.

The results clearly demonstrate that peptide Y₂₁ (SEQ ID NO:15) is an effective peptidomimetic of neurotrophic actions of T. cruzi on TrkA. The results also demonstrate that peptide Y₂₁ (SEQ ID NO:15), in addition to its usefulness in ascertaining the molecular mechanism of PDNF activation of TrkA, can serve as a basis for the development of therapeutics (e.g., small molecule therapeutics) to treat disorders in which Trk receptors play a role, such as peripheral diabetic neuropathies (Ebadi et al., Neurochem. Int. 30:347-374 (1997)) and treatment of neuronal damage (Hughes et al., Neuroscience 78:1037-1049 (1997)).

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A neurotrophic peptide consisting of the amino acid sequence of peptide C19Y21 (SEQ ID NO:15) or a neurotrophic variant thereof.

2. A macromolecular complex comprising a macromolecule that is covalently bonded to a neurotrophic peptide.

3. The macromolecular comples of claim 2 wherein said neurotrophic peptide comprises the amino acid sequence of peptide C19Y21 (SEQ ID NO:15) or a neurotrophic variant thereof.

4. The macromolecular complex of claim 2, wherein said macromolecule is a protein, a peptide, a nucleic acid, or a polyethylene glycol.

5. A method of activating TrkA receptor comprising combining a cell that expresses TrkA receptor with a neurotrophic peptide of claim 1 or a macromoleculer complex of claim 2 under conditions suitable for binding to said TrkA receptor.

6. A method for identifying an agent that activates TrkA receptor, comprising combining a cell that expresses TrkA receptor and an agent to be tested under conditions suitable for binding of a ligand to said TrkA receptor, and detecting or measuring TrkA receptor activation, wherein TrkA receptor activation relative to a suitable control is indicative that the agent activates TrkA receptor, with the proviso that the agent is not nerve growth factor.

7. The method of claim 6 wherein said agent competitively inhibits binding of TS to TrkA receptor but does not competitively inhibit binding of nerve growth factor to TrkA receptor.

8. The method of claim 7 wherein said agent further competitively inhibits binding of a peptide consisting of the amino acid sequence of SEQ ID NO:15 to TrkA receptor.

9. The method of claim 6 wherein TrkA receptor activation is detected or measured by detecting or measuring agent-induced TrkA dimerization, agent-induced TrkA phosphorylation, agent-induced Erk phosphorylation, agent-induced Akt phosphorylation, agent-induced CREB phosphorylation or agent-induced cAMP response element-depended transcription.


SEQUENCE LISTING (35 sequences)

SEQ ID NO: 1 - DNA, 2133 bp, Trypanosoma cruzi
tgttccccttttctcttcccaactttctccggcggcaatccccctgcaaagagacgatcttgacaccattgttttaggcataatagaagttctacaaacaacgcccgaaggacacacaggcaggcaccgactaccatggggaaaacagtcgttgtggccagtaggatgttctggctaatgtttttcgtgccgcttcttcttgcgatctgccccagcgagcccgcgtacgccttggcacccggatcgagccgagttgagctgtttaagcgtaagaattcgacggtgccgtttgaagacaaggccggcaaagtcaccgagcgggttgtccactcgttccgcctccccgcccttgttaatgtggacggggtgatggttgccatcgcggacgctcgctacgacacatccaatgacaactccctcattgatacggtggcgaagtacagcgtggacgatggggagacgtgggagacccaaattgccatcaagaacagccgtgtatcgtctgtttctcgtgtggtggatcccaccgtgattgtgaagggcaacaagctttacgtcctggttggaagctactatagttcgagaagctactggtcgtcgcatggtgatgcgagagactgggatattctgcttgccgttggtgaggtcacgaagtccactgcgggcggcaagataactgcgagtatcaaatgggggagccccgtgtcactgaagaagttttttccggcagaaatggaaggcatgcacacaaatcaatttcttggcggcgcgggtgttgccattgtagcgtccaacgggaatcttgtgtaccctgtgcaggttacgaacaaaaagaagcaagttttctccaagatcttctactcggaagatgatggcaagacgtggaagtttgggaagggtaggagcgattttggctgctctgaacctgtggcccttgagtgggaggggaagctcatcataaacacccgagttgactggaaacgccgtctggtgtacgagtccagtgacatggagaaaccgtgggtggaggctgtcggaaccgtctcgcgtgtgtggggcccctcaccaaaatcgaaccagcccggcagtcagagcagcttcactgccgtgaccatcgaaggaatgcgtgtgatgctcttcacacacccgctgaattttaagggaaggtggctgcgcgaccgactgaacctctggctgacggataaccagcgcatttataacgttgggcaagtatccattggtgatgaaaattccgcctacagctccgtcctgtacaaggatgataagctgtactgtttgcatgagatcaacacggacgaggtgtacagccttgtttttgcacgcctggttggcgagctacggatcattaaatcagtgctgcggtcctggaagaattgggacagccacctgtccagcatttgcacccctgctgatccagccgcttcgtcgtcagagagtggttgtggtcccgctgtcaccacggttggtcttgttggctttttgtccggcaacgcctcccaaaacgtatgggaggatgcgtaccgctgcgtcaacgcaagcacggcaaatgcggagagggttcggaacggtttgaagtttgcgggggttggcggaggagcgctttggccggtgagccagcaggggcagaatcagcggtatcgttttgcaaaccacgcgttcacgctggtggcgtcggtgacgattcacgaggctccgagggccgcgagtcccttgctgggtgcgagcctggactcttctggcggcaaaaaactcctggggctctcgtacgacgagaagcaccagtggcagccaatatacggatcaacgccggtgacgccgacgggatcgtgggagacgggtaaaaggtaccacttggttcttacgatggcgaataaaattggctccgtgtacattgatggagaacttctggagggttcaggacagaccgttgtgccagacgggaggacgcctgacatctcccacttctacgttggcgggtataaaaggagtgatatgccaaccataagccacgtgacggtgaataatgttcttctttacaaccgacagctgaataccgaggagatcaggaccttgttcttgagccaggaccttattggcacggaagcacacatg

SEQ ID NO: 2 - protein, 666 aa, Trypanosoma cruzi
MGKTVVVASRMFWLMFFVPLLLAICPSEPAYALAPGSSRVELFKRKNSTVPFEDKAGKVTERVVHSFRLPALVNVDGVMVAIADARYDTSNDNSLIDTVAKYSVDDGETWETQIAIKNSRVSSVSRVVDPTVIVKGNKLYVLVGSYYSSRSYWSSHGDARDWDILLAVGEVTKSTAGGKITASIKWGSPVSLKKFFPAEMEGMHTNQFLGGAGVAIVASNGNLVYPVQVTNKKKQVFSKIFYSEDDGKTWKFGKGRSDFGCSEPVALEWEGKLIINTRVDWKRRLVYESSDMEKPWVEAVGTVSRVWGPSPKSNQPGSQSSFTAVTIEGMRVMLFTHPLNFKGRWLRDRLNLWLTDNQRIYNVGQVSIGDENSAYSSVLYKDDKLYCLHEINTDEVYSLVFARLVGELRIIKSVLRSWKNWDSHLSSICTPADPAASSSESGCGPAVTTVGLVGFLSGNASQNVWEDAYRCVNASTANAERVRNGLKFAGVGGGALWPVSQQGQNQRYRFANHAFTLVASVTIHEAPRAASPLLGASLDSSGGKKLLGLSYDEKHQWQPIYGSTPVTPTGSWETGKRYHLVLTMANKIGSVYIDGELLEGSGQTVVPDGRTPDISHFYVGGYKRSDMPTISHVTVNNVLLYNRQLNTEEIRTLFLSQDLIGTEAHM

SEQ ID NO: 3 - DNA, 34 bp, Artificial Sequence
  Synthetic primer
ggaattccatatggcacccggatcgagccgagtt

SEQ ID NO: 4 - DNA, 32 bp, Artificial Sequence
  Synthetic Primer
ccgctcgaggctcaagaacaaggtcctgatcg

SEQ ID NO: 5 - DNA, 29 bp, Artificial Sequence
  Synthetic primer
gggaattcggttgccaatcgcggacgctc

SEQ ID NO: 6 - DNA, 26 bp, Artificial Sequence
  Synthetic primer
cccctcgagatttgccgtgcttgcgt

SEQ ID NO: 7 - DNA, 31 bp, Artificial Sequence
  Synthetic primer
cccctcgagccgacaaaaagccaacaaagac

SEQ ID NO: 8 - DNA, 25 bp, Artificial Sequence
  Synthetic primer
agatgaagactccgcgcccctgagg

SEQ ID NO: 9 - DNA, 23 bp, Artificial Sequence
  Synthetic primer
ccaggtatgcacccagagtgatg

SEQ ID NO: 10 - DNA, 24 bp, Artificial Sequence
  Synthetic primer
cggagtcaacggatttggtcgtat

SEQ ID NO: 11 - DNA, 24 bp, Artificial Sequence
  Synthetic primer
agccttctccatggtggtgaagac

SEQ ID NO: 12 - protein, 45 aa, Artificial Sequence
  Synthetic peptide
QPLRRQRVVVVPLSPRLVLLAFCRQRLPLKRMGGSYRCVNASTAN

SEQ ID NO: 13 - protein, 21 aa, Artificial Sequence
  Synthetic peptide
RQRLPKRMGGSYRCVNASTAH

SEQ ID NO: 14 - protein, 14 aa, Artificial Sequence
  Synthetic peptide
RQRLPKRMGGSYRC

SEQ ID NO: 15 - protein, 21 aa, Artificial Sequence
  Synthetic peptide
GNASQNVWEDAYRCVNASTAN

SEQ ID NO: 16 - protein, 14 aa, Artificial Sequence
  Synthetic peptide
GNASQNYWEDAYRC

SEQ ID NO: 17 - protein, 7 aa, Artificial Sequence
  Synthetic peptide
VNASTAN

SEQ ID NO: 18 - protein, 10 aa, Artificial Sequence
  Synthetic peptide
YSVDDGETWE

SEQ ID NO: 19 - protein, 12 aa, Artificial Sequence
  Synthetic peptide
DSSAHGTPSTPA

SEQ ID NO: 20 - DNA, 27 bp, Artificial Sequence
  Synthetic primer
gcccatggcacccgatcgagccgagtt

SEQ ID NO: 21 - DNA, 20 bp, Artificial Sequence
  Synthetic primer
cggaattttcatcaccaatg

SEQ ID NO: 22 - DNA, 24 bp, Artificial Sequence
  Synthetic primer
gttccgaacggtttgaagtttgcg

SEQ ID NO: 23 - DNA, 24 bp, Artificial Sequence
  Synthetic primer
gttccgaacggtttgaagtttgcg

SEQ ID NO: 24 - DNA, 23 bp, Artificial Sequence
  Synthetic primer
atgaactccttctccacaagcgc

SEQ ID NO: 25 - DNA, 22 bp, Artificial Sequence
  Synthetic primer
gaagagccctcaggctggactg

SEQ ID NO: 26 - protein, 24 aa, Artificial Sequence
  Synthetic peptide
DSSAHGAPSTPADSSAHGTPSTPV

SEQ ID NO: 27 - protein, 36 aa, Artificial Sequence
  Synthetic peptide
DSSAHGAPSTPADSSAHGTPSTPVDSSAHGTPSTPA

SEQ ID NO: 28 - protein, 48 aa, Artificial Sequence
  Synthetic peptide
DSSAHGAPSTPADSSAHGTPSTPVDSSAHGTPSTPADSSAHSTPSTPA

SEQ ID NO: 29 - protein, 60 aa, Artificial Sequence
  Synthetic peptide
DSSAHGAPSTPADSSAHGTPSTPVDSSAHGTPSTPADSSAHSTPSTPADSSAHSTPSTPA

SEQ ID NO: 30 - protein, 200 aa, Trypanosoma cruzi
KKKQVFSKIFYSEDEGKTWKFGEGRSDFGCSEPVALEWEGKLIINTRVDYRRRLVYESSDMGNSWVEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTP

SEQ ID NO: 31 - protein, 200 aa, Trypanosoma cruzi
KKKQVFSKIFYSEDDGKTWKFGEGRSAFGCSEAVALEWEGKLIINTRVDYRRRLVYESSDMGNTWLEAVGTLSRVWGPSPKSNQPGSQSSFTAVTIEGMRVMLFTHPLNFKGRWLRDRLNLWLTDNQRIYNVGQVSIGDENSAHSSVLYKDDKLYCLHEINSNEVYSLVFARLVGELRIIKSVLQSWKNWDSHLSSICTP

SEQ ID NO: 32 - protein, 12 aa, Artificial Sequence
  Synthetic peptide
DSSAHGAPSTPA

SEQ ID NO: 33 - DNA, 5403 bp, Trypanosoma cruzi
aaagaccgttggaagaagaaagaaggttccggagcgtggccaccaccaacgatgaactgccacaattgcgtgctgtccgcgggcggtacccggcgctttgagcccacggcgacttgtgtgttcccctttctcttcccactttctccgcggcaatccccctgcaaagagacgatcttgacaccattgttttaggcataatagaagttctacaaacaacgcccgaaggacacacaggcaggcaccgactacgatggggaaaacagtcgttgtggccagtaggatgttctggctaatgtttttcgtgccgcttcttcttgcgatctgccccagcgagcccgcgtacgccctggcacccggatcgagccgagttgagggtttaagcgtaagaattcgacggtgccgtttgaagacaaggccggcaaagtcaccgagcgggttgtccactcgttccgcttccccgcccttgttaatgtggacggggtgatggttgccatcgcggacgctcgctacgaaacatccagtgaaaactccctcattgatacggtggcgaagtacagcgtggacgatggggagacgtgggagacccaaattgccatcaagaacagccgtgtatcgtctgtttctcgtgtggtggatcccaccgtgattgtgaagggcaacaagctttacgtcctggttggaagctactatagttcgagaagctactggtcgtcgcatggtgatgcgagagactgggatattctgcttgccgttggtgaggtcacgaagtccactgcgggcggcaagataactgcgagtatcaaatgggggagccccgtgtcactgaagaagttttttccggcagaaatggaaggcatgcacacaaatcaatttcttggcggcgcgggtgttgccattgtagcgtccaacgggaatcttgtgtaccctgtgcaggttacgaacaaaaggaagcaagttttctccaagatcttctactcggaagatgatggcaagacgtggaagtttgggaagggtaggagcgattttggctgctctgaacctgtggcccttgagtgggaggggaagctcatcataaacacccgagttgactggaaacgccgtctggtgtacgagtccagtgacatggagaaaccgtgggtggaggctgtcggaaccgtctcgcgtgtgtggggcccctcaccaaaatcgaaccagcccggcagtcagacgagcttcactgccgtgaccatcgaaggaatgcgtgtgatgctcttcacacacccgctgaattttaagggaaggtgcgtgcgcgaccgactgaacctctggctgacggataaccagcgcatttataacgttgggcaagtatccattggtgatgaaaattccgcctacagctccgtcctgtacaaggatgataagctgtactgtttgcatgagatcaacacggacgaggtgtacagccttgtttttgcacgcctggttggcgagctacggatcattaaatcagtgctgcggtcctggaagaattggacagccacctgtccagcatttgcacccctgctgatccagccgcttcgtcgtcagagagtggttgtggtcccgctgtcaccacggttggtcttgttggctttttgtcggcaacgcctcccaaaacgtatgggaggatcgtaccgctgcgtcaacgcaagcacggcaaatgcggagagggttcggaacggtttgaagtttgcgggggttggcggaggagcgctttggccggtgagccagcaggggcagaatcagcggtatcgttttgcaaaccacgcgttcacgctggtggcgtcggtgacgattcacgaggctccgagggccgcgagtcccttgctgggtgcgagcctggactcttctggcggcaaaaaactcctggggctctcgtacgacgagaagcaccagtggcagccaatatacggatcaacgccggtgacgccgacgggatcgtgggagacgggtaaaaggtaccacttggttcttacgatggcgaataaaattggctccgtgtacattgatggagaacttctggagggttcaggacagaccgttgtgccagacgggaggacgcctgacatctcccacttctacgttggcgggtataaaaggagtgatatgccaaccataagccacgtgacggtgaataatgttcttctttacaaccgacgacagctgaataccgaggagatcaggaccttgttcttgagccaggaccttattggcacggaagcacacatggacagcagcagcgacagcagtgcccacagtacgccctcaactcccgctgacagcagtgcccacagtacgccctcaactcccgttgacagcagtgcccacagtacgccctcgactcccgctgacagcagtgcccacggtacgccctcaactcccgttgacagcagtgcccacggtacgccctcaactcccgctgacagcagtgcccacggtacgccctcaactcccgttgacagcagtgcccacagtacgccctcaactcccgttgacagcagtgcccacagtacgccctcaactcccgttgacagcagtgcccacggtgcgccctcaactcccgctgacagcagtgcccacggtacgccctcgactcccgttgacagcagtgcccacggtacgccctcgactcccgctgacagcagtgcccacagtacgccctcgactcccgctgacagcagtgcccacagtacgccctcgactcccgctgacagcagtgcccacagtacgccctcgactcccgttgacagcagtgcccacggtacgccctcgactcccgctgacagcagtgcccacagtacgccctcgactcccgctgacagcagtgcccacggtacgccctcaactcccgttgacagcagtgcccacagtacgccctcgactcccgttgacagcagtgcccacggtacgccctcaactcccgttgacagcagtgcccacagtacgccctcgactcccgttgacagcagtgcccacggtacgccctcaactcccgttgacagcagtgcccacagtacgccctcgactcccgctgacagcagtgcccacagtacgccctcaactcccgctgacagcagtgcccacggtacgccctcaactcccgttgacagcagtgcccacagtacgccctcgactcccgctgacagcagtgcccacagtacgccctcaactcccgttgacagcagtgcccacagtacgccctcaactcccgctgacagcagtgcccacggtacgccctcaactcccgttgacagcagtgcccacggtacgccctcgactcccgctgacagcagtgcccacagtacgccctcgactcccgctgacagcagtgcccacagtacgccctcgactcccgctgacagcagtgcccacagtacgccctcaactcccgttgacagcagtgcccacagtacgccctcaactcccgctgacagcagtgcccacagtacgccctcaactcccgctgacagcagtgcccacagtacgccctcgactcccgctgacagcagtgcccacagtacgccctcaactcccgttgacagcagtgcccacagtacgccctcaactcccgctgacagcagtgcccacggtacgccctcgactcccgctgacagcagtgcccacagtacgccctcgactcccgttgacagcagtgcccacagtacgccctcgactcccgctgacagcagtgcccacggtacgccctcgactcccgctgacagcagtgcccacagtacgccctcgactcccgctgacagcagtgcccacggtacgccctcgactcccgctgacagcagtgcccacagtacgccctcaactcccgctggcagcagcgccaatggtacggttctgattttgcccgatggcgctgcactttcgaccttttcgggcggagggcttcttctgtgtgcgtgtgctttgctgctgcacgtgttttttatggcagttttctgatgtagtgagagagtctcctaacaaatgtagataaattcataattgtggtgtgaaccgtttgggtaaatgtgtgtgtgcgctctcataaggaaatgatttccagtaatgttttttttttgttctcgaacattttgaataaatctgcagacagatggggacgcgtaatttgaatttgtttttcagcgttcttttgtcactggccccttgtttaagtggaaccgcgttgcaatgcggcgagggcatttctctgttttgatttccttctttttctcctttgtgtttcttcaatttgacggtttgcacgctgtgcggtggagcgttttcccttgtgaaataagggccaactgcttcacagtggcacgagggcgctcaagagatccgcgggtcgccagtgactcactttgtgtggcgcagctcgaggaggtgtctggctgctgtgggggcctcgatggttgccacttcgcgagtttgcaacgagcgtgcttctcgcggagggagcaggcgaaatattttgttttttttttttgttttttttgtttttttgtttttgtgtgtgtgtgtaagttttggttcagtctcccttgaactgggggacgttgggcttaatggaccaaactctgattcccctaaaacttcttttgttggttttcttttgtttttgtttttgtgctgctgatttgcacgctttctcactgtcaccgaagcgcggcggcggtgtttgagtgccccctcacgctgctgctgtggaatttgcgttgcttgcggacatttctgttgggtcgcattgctttctacttcgttttttatttttgtggtttggtggaggggagtgtgcagcagggggcgggccgagatgcctgtggagacagcgacgttgcggggactctctctcggcctcgtcattcaacaatccattgcgcagcaggttgccacgaacaccagcaccaatatttgttcgttttcccactattaccggcgcgtctagccgcacgatgccatctgggtgccgaggaggcggttgagcagcggaaaaggcttcctgctatgaagcgactgccattgagagaacttttagctgcgtggatcttcctcaatgcccagccgttggcgcgcagcggaggtgcctgggcattctaggagcagatggcgaaaggtttcctgcgcgtcaactggcgtgtctgtggaggttggctatcctcagtcgggagaccgcctcctggcaccacagaacgggtagcggtagtgtcttggcgaatagtacaacgccacttgttgctgactgggcagtaaagcatgtcagcgggtccgtgtgccatacgggcgcattccatgttccgtgtgttgtccggttgccatggtctgcgtcgcatgctgagccgcaggctcgtcaacatgcactccacaatgtccgtaagaaaactcccggtgcac

SEQ ID NO: 34 - protein, 1162 aa, Trypanosoma cruzi
MVAIADARYETSSENSLIDTVAKYSVDDGETWETQIAIKNSRVSSVSRVVDPTVIVKGNKLYVLVGSYYSSRSYWSSHGDARDWDILLAVGEVTKSTAGGKITASIKWGSPVSLKKFFPAEMEGMHTNQFLGGAGVAIVASNGNLVYPVQVTNKRKQVFSKIFYSEDDGKTWKFGKGRSDFGCSEPVALEWEGKLIINTRVDWKRRLVYESSDMEKPWVEAVGTVSRVWGPSPKSNQPGSQTSFTAVTIEGMRVMLFTHPLNFKGRCVRDRLNLWLTDNQRIYNVGQVSIGDENSAYSSVLYKDDKLYCLHEINTDEVYSLVFARLVGELRIIKSVLRSWKNWTATCPAFAPLLIQPLRRQRVVVVPLSPRLVLLAFCRQRLPKRMGGSYRCVNASTANAERVRNGLKFAGVGGGALWPVSQQGQNQRYRFANHAFTLVASVTIHEAPRAASPLLGASLDSSGGKKLLGLSYDEKHQWQPIYGSTPVTPTGSWETGKRYHLVLTMANKIGSVYIDGELLEGSGQTVVPDGRTPDISHFYVGGYKRSDMPTISHVTVNNVLLYNRRQLNTEEIRTLFLSQDLIGTEAHMDSSSDSSAHSTPSTPADSSAHSTPSTPVDSSAHSTPSTPADSSAHGTPSTPVDSSAHGTPSTPADSSAHGTPSTPVDSSAHSTPSTPVDSSAHSTPSTPVDSSAHGAPSTPADSSAHGTPSTPVDSSAHGTPSTPADSSAHSTPSTPADSSAHSTPSTPADSSAHSTPSTPVDSSAHGTPSTPADSSAHSTPSTPADSSAHGTPSTPVDSSAHSTPSTPVDSSAHGTPSTPVDSSAHSTPSTPVDSSAHGTPSTPVDSSAHSTPSTPADSSAHSTPSTPADSSAHGTPSTPVDSSAHSTPSTPADSSAHSTPSTPVDSSAHSTPSTPADSSAHGTPSTPVDSSAHGTPSTPADSSAHSTPSTPADSSAHSTPSTPADSSAHSTPSTPVDSSAHSTPSTPADSSAHSTPSTPADSSAHSTPSTPADSSAHSTPSTPVDSSAHSTPSTPADSSAHGTPSTPADSSAHSTPSTPVDSSAHSTPSTPADSSAHGTPSTPADSSAHSTPSTPADSSAHGTPSTPADSSAHSTPSTPAGSSANGTVLILPDGAALSTFSGGGLLLCACALLLHVFFMAVF

SEQ ID NO: 35 - protein, 14 aa, Artificial Sequence
  Synthetic peptide
GNASQNVWEDAYRC